SEQUENCE LISTING (13 sequences)

SEQ ID NO: 1 - DNA, 1753 bp, Newcastle Disease Virus
  misc_feature (1)..(1753): See Figures 1a and 1b.
atggacagagcagtttcacaagtggccctagagaatgatgagagggaagccaagaatacctggaggcttatattcagaatagccatcttattccttactgtggtcaccctagcaatctctgttgcatccctcctctattctatgggagcaagcaccccctcagacttggtgggcatacccacaagaatctctagggcagaagaaaaaatcaccagtacccttggctccaaccaagatgttgtggacagaatctacaaacaggtggcacttgaaagtccacttgcattactcaacacagagactaccatcatgaatgcaattaccagcctatcctatcaaattaatggggctgccaacaattcaggttggggagccccaattcatgatccagactatattggaggtattggcaaagagcttattgtagatgatgcttcagatgttacatctttctatccttcagctttccaggaacacctgaatttcattcctgcacccacaactgggagtgggtgcactagaataccctcatttgacatgagtgctacacactactgctacacacataatgttattctctctggctgtagggaccactctcactcttatcaatacttagctcttggagttctcagaacatctgctactggtagagtctttttctcaactcttaggagtatcaacctagatgatacacaaaataggaaaagttgctctgtatctgctacacctttgggctgtgatatgctatgcagtaaagtaacagaaactgaagaagaggactataattctgctgtccctacaaggatggtgcatggcagattgggttttgatggtcaatatcatgaaaaagatttggatgtcactacattgtttggggattgggtagctaattacccaggagttggaggtggtagcttcattgactccagagtctggttctctgtctatggtggtttaaaacctaacagtcctagtgatactgtgcaagagggaaagtatgttatctacaagaggtataatgatacttgtcctgatgaacaggattaccagattaggatggctaagtcatcatacaaaccaggaagatttggaggtaagaggatacaacaagctattttgagtattaaggttagcacatcattgggagaggacccagtccttactgttccaccaaacactgtaacactcatgggagctgagggaaggattttaactgttggtactagccattttctttatcagagaggaagttcctattttagcccagcattactgtatccaatgactgtgagcaacaagacagctacattacattcaccatatacttttaatgcttttacaagacctggatcaattccttgccaggcttcagctagatgtccaaattcatgtgtgactggagtttacactgatccttaccctttgatattttacagaaatcataccttgagaggggtttttggaacaatgttggatggtgttcaagctaggctcaatcctgcctctgctgtttttgattctacatcaagatcaagaataaccagggtttcctctagttccactaaggcagcatatactacctccacatgtttcaaagttgtaaagactaacaaaacttattgtctgagcatagctgagatctctaacactctttttggggagttcagaattgttccacttttggtggaaattctgaaggatgatggtgtaagggaagcaagatctggttaagtcttcaggtaccgagctc

SEQ ID NO: 2 - protein, 577 aa, Newcastle Disease Virus
  misc_feature: See Figures 1a and 1b.
MDRAVSQVALENDEREAKNTWRLIFRIAILFLTVVTLAISVASLLYSMGASTPSDLVGIPTRISRAEEKITSTLGSNQDVVDRIYKQVALESPLALLNTETTIMNAITSLSYQINGAANNSGWGAPIHDPDYIGGIGKELIVDDASDVTSFYPSAFQEHLNFIPAPTTGSGCTRIPSFDMSATHYCYTHNVILSGCRDHSHSYQYLALGVLRTSATGRVFFSTLRSINLDDTQNRKSCSVSATPLGCDMLCSKVTETEEEDYNSAVPTRMVHGRLGFDGQYHEKDLDVTTLFGDWVANYPGVGGGSFIDSRVWFSVYGGLKPNSPSDTVQEGKYVIYKRYNDTCPDEQDYQIRMAKSSYKPGRFGGKRIQQAILSIKVSTSLGEDPVLTVPPNTVTLMGAEGRILTVGTSHFLYQRGSSYFSPALLYPMTVSNKTATLHSPYTFNAFTRPGSIPCQASARCPNSCVTGVYTDPYPLIFYRNHTLRGVFGTMLDGVQARLNPASAVFDSTSRSRITRVSSSSTKAAYTTSTCFKVVKTNKTYCLSIAEISNTLFGEFRIVPLLVEILKDDGVREARSG

SEQ ID NO: 3 - DNA, 1647 bp, Avian Influenze Virus
  misc_feature (1)..(1647): See Figure 10
gaccaaatctgcatcggttatcatgcaaacaattcaacaaaacaagttgacacaatcatggagaagaatgtgacggtcacacatgctcaagatatactggaaaaagagcacaacgggaaactctgcagtctcaaaggagtgaggcccctcattctgaaggattgcagtgtggctggatggcttcttgggaacccaatgtgtgatgagttcctaaatgtaccggaatggtcatatattgtagagaaggacaatccaaccaatggcttatgttatccgggagacttcaatgattatgaagaactgaagtatttaatgagcaacacaaaccattttgagaaaattcaaataatccctaggaactcttggtccaatcatgatgcctcatcaggagtgagctcagcatgcccatacaatggtaggtcttcctttttcaggagtgtggtgtggttgatcaagaagagtaatgtatacccaacaataaagaggacctacaataacaccaatgtagaggaccttctgatattgtggggaatccatcaccctaatgatgcagcggaacaaacggaactctatcagaactcgaacacttatgtgtctgtaggaacatcaacactaaatcagaggtcaattccagaaatagctaccaggcccaaagtgaatggacaaagtggaagaatagaatttttctggacaatactaaggccgaacgatgcaatcagctttgaaagtaatgggaactttatagctcctgaatatgcatacaagatagttaaaaagggagattcagcaatcatgagaagcgaactggagtatggcaactgtgataccaaatgtcagaccccagtgggtgctataaattccagtatgccttttcacaatgttcatccccttaccattggagagtgtcccaaatatgtcaaatcagataaactggtccttgcaacaggactgaggaacgtgcctcagagagaaacaagaggtctgtttggagcaatagcaggattcatagaaggggggtggcaaggaatggtagatggatggtatggttaccatcatagcaacgagcagggaagtggatatgctgcagacaaagagtccactcagaaagcaatcgacgggatcaccaataaagtcaactcaatcattgacaaaatgaacactcaattcgaagccgttgggaaagaattcaacaacttagaaaggagaatagaaaatttgaataagaaaatggaagatggatttctagatgtatggacttacaatgcagaacttctggtgctcatggaaaatgaaagaactctggatttccatgattcatatgtcaagaacctatacgataaggtccgactccagctgagagataatgcaaaagaattgggcaatgggtgtttggagttctcccacaaatgtgacaatgaatgcatggaaagtgtgagaaacggaacgtatgactatccacaatactcagaagaatcaaggctgaacagagaggaaatagatggagtcaaattggagtcaatgggcacctatcagatactatcaatttactcaacagtggcgagttccctagcactggcaatcatggtagctggtctgtctttttggatgtgctccaatggatcattgcaatgcagaatttgcatctag

SEQ ID NO: 4 - protein, 548 aa, Avian Influenze Virus
  misc_feature: See Figure 10.
DQICIGYHANNSTKQVDTIMEKNVTVTHAQDILEKEHNGKLCSLKGVRPLILKDCSVAGWLLGNPMCDEFLNVPEWSYIVEKDNPTNGLCYPGDFNDYEELKYLMSNTNHFEKIQIIPRNSWSNHDASSGVSSACPYNGRSSFFRSVVWLIKKSNVYPTIKRTYNNTNVEDLLILWGIHHPNDAAEQTELYQNSNTYVSVGTSTLNQRSIPEIATRPKVNGQSGRIEFFWTILRPNDAISFESNGNFIAPEYAYKIVKKGDSAIMRSELEYGNCDTKCQTPVGAINSSMPFHNVHPLTIGECPKYVKSDKLVLATGLRNVPQRETRGLFGAIAGFIEGGWQGMVDGWYGYHHSNEQGSGYAADKESTQKAIDGITNKVNSIIDKMNTQFEAVGKEFNNLERRIENLNKKMEDGFLDVWTYNAELLVLMENERTLDFHDSYVKNLYDKVRLQLRDNAKELGNGCLEFSHKCDNECMESVRNGTYDYPQYSEESRLNREEIDGVKLESMGTYQILSIYSTVASSLALAIMVAGLSFWMCSNGSLQCRICI

SEQ ID NO: 5 - DNA, 28 bp, Synthetic sequence
  misc_feature (1)..(28): PCR primer, CVM-Asc, used to end-tailor the constitutive cassava vein mosaic virus (CsVMV) promoter on pCP H.
atggcgcgccagaaggtaattatccaag

SEQ ID NO: 6 - DNA, 24 bp, Synthetic sequence
  misc_feature (1)..(24): PCR primer, CVM-Xho, used to end-tailor the cassava vein mosaic virus (CsVMV) promoter on pCP H.
atctcgagccatggtttggatcca

SEQ ID NO: 7 - DNA, 25 bp, Synthetic sequence
  misc_feature (1)..(25): Mutagenic primer used to create a Nco I site.
tgccatggtgatgtgtggtctacaa

SEQ ID NO: 8 - DNA, 23 bp, Synthetic sequence
  misc_feature (1)..(23): Forward primer complementary to the 5′ region.
gatctgacaagtcaagaaaattg

SEQ ID NO: 9 - DNA, 23 bp, Synthetic sequence
  misc_feature (1)..(23): Mutagenic primer used to create a XhoI I site.
agctcgagctgtgtgagtgagtg

SEQ ID NO: 10 - DNA, 1368 bp, Infectious Bursal Disease Virus
  misc_feature (1)..(1368): See Figure 14.
atgaccaacctccaagatcaaactcaacagattgttcccttcatacgcagccttctcatgccaaccactggacctgcttccattcctgatgacaccttggagaagcacactctccgctctgagacctcaacctacaacttgactgttggtgacactggctctgggttgattgtctttttccctgggttccctggctccattgtgggtgctcactacacattgcagtccaatggcaactacaagtttgatcaaatgctcttgactgcccagaatcttccagcctcctacaactattgccgtcttgtgtctcgctccctcacagtgaggtcctcaacactccctggtggagtgtatgcactcaatggcaccatcaacgcagtgactttccaaggaagcctttcagaattgactgatgtgagctacaatgggttgatgtctgcaacagccaacatcaatgacaagattgggaatgtccttgttggagaaggagtcaccgtcctctcactcccaacatcctatgatcttggctatgtgagacttggtgatcccattcctgccataggacttgatcccaaaatggttgccacatgtgacagctctgatcgtccaagggtttacaccatcacagcagctgatgactaccaattctcctcacagtaccaagctggtggagtcaccatcacactcttctcagccaacatagatgccatcacaagcctcagcattggtggagaacttgtctttcagacatctgtccaagggctcatccttggtgccaccatctacttgattggctttgatggcactgctgtcatcaccagagcagtggctgcagacaatgggctcacagctggcactgacaacctcatgccattcaacattgtgattcccacctctgagatcacccagccaatcacttccatcaagttggagatagtgacctcaaagtccggtggacaagctggtgatcagatgtcctggtctgcatctgggagcttggctgtgaccattcatggtggcaactaccccggagccctcagacctgtgactttggttgcctatgaacgcgttgcaactggctctgttgtcactgttgctggtgtcagcaactttgagttgatcccaaatcctgaacttgcaaagaacttggtcacagagtatggaaggtttgaccctggtgccatgaactacacaaaattgatcctctcagagagggacagacttggcatcaagactgtttggccaaccagagagtacactgacttccgcgagtacttcatggaggttgctgacctcaacagccctctcaagatagctggagcctttggtttcaaagacatcataagggctattcgtcgcatcgctgtt

SEQ ID NO: 11 - DNA, 1425 bp, Infectious Bursal Disease Virus
  misc_feature: Polynucleotide (DNA) encoding a variation of E/91 VP2 (structural protein from Infectious Bursal Disease Virus)
agatctgaagacaacatgaccaacctccaagatcaaactcaacagattgttcccttcatacgcagccttctcatgccaaccactggacctgcttccattcctgatgacaccttggagaagcacactctccgctctgagacctcaacctacaacttgactgttggtgacactggctctgggttgattgtctttttccctgggttccctggctccattgtgggtgctcactacacattgcagtccaatggcaactacaagtttgatcaaatgctcttgactgcccagaatcttccagcctcctacaactattgccgtcttgtgtctcgctccctcacagtgaggtcctcaacactccctggtggagtgtatgcactcaatggcaccatcaacgcagtgactttccaaggaagcctttcagaattgactgatgtgagctacaatgggttgatgtctgcaacagccaacatcaatgacaagattgggaatgtccttgttggagaaggagtcaccgtcctctcactcccaacatcctatgatcttggctatgtgagacttggtgatcccattcctgccataggacttgatcccaaaatggttgccacatgtgacagctctgatcgtccaagggtttacaccatcacagcagctgatgactaccaattctcctcacagtaccaagctggtggagtcaccatcacactcttctcagccaacatagatgccatcacaagcctcagcattggtggagaacttgtctttcagacatctgtccaagggctcatccttggtgccaccatctacttgattggctttgatggcactgctgtcatcaccagagcagtggctgcagacaatgggctcacagctggcactgacaacctcatgccattcaacattgtgattcccacctctgagatcacccagccaatcacttccatcaagttggagatagtgacctcaaagtccggtggacaagctggtgatcagatgtcctggtctgcatctgggagcttggctgtgaccattcatggtggcaactaccccggagccctcagacctgtgactttggttgcctatgaacgcgttgcaactggctctgttgtcactgttgctggtgtcagcaactttgagttgatcccaaatcctgaacttgcaaagaacttggtcacagagtatggaaggtttgaccctggtgccatgaactacacaaaattgatcctctcagagagggacagacttggcatcaagactgtttggccaaccagagagtacactgacttccgcgagtacttcatggaggttgctgacctcaacagccctctcaagatagctggagcctttggtttcaaagacatcataagggctattcgtcgcatcgctgtttgagtagttagcttaatcacctagagctcggtcaccagatct

SEQ ID NO: 12 - protein, 456 aa, Infectious Bursal Disease Virus
  misc_feature: Polypeptide variation of E/91 VP2 (structural protein from Infectious Bursal Disease Virus) encoded by SEQ ID NO 11.
MTNLQDQTQQIVPFIRSLLMPTTGPASIPDDTLEKHTLRSETSTYNLTVGDTGSGLIVFFPGFPGSIVGAHYTLQSNGNYKFDQMLLTAQNLPASYNYCRLVSRSLTVRSSTLPGGVYALNGTINAVTFQGSLSELTDVSYNGLMSATANINDKIGNVLVGEGVTVLSLPTSYDLGYVRLGDPIPAIGLDPKMVATCDSSDRPRVYTITAADDYQFSSQYQAGGVTITLFSANIDAITSLSIGGELVFQTSVQGLILGATIYLIGFDGTAVITRAVAADNGLTAGTDNLMPFNIVIPTSEITQPITSIKLEIVTSKSGGQAGDQMSWSASGSLAVTIHGGNYPGALRPVTLVAYERVATGSVVTVAGVSNFELIPNPELAKNLVTEYGRFDPGAMNYTKLILSERDRLGIKTVWPTREYTDFREYFMEVADLNSPLKIAGAFGFKDIIRAIRRIAV

SEQ ID NO: 13 - DNA, 42 bp, Infectious Bursal Disease Virus
  misc_feature (1)..(42): DNA sequence encoding translation termination (“Stop”) codons, used to terminate translation of inadvertant open reading frames following DNA integration during transformation (includes Sac I BstE II, and Bgl II restriction enzyme recogniton sites).
tgagtagttagcttaatcacctagagctcggtcaccagatct

(19) United States
(12) Patent Application Publication
(10) Pub. No.: US 2004/0268442 A1
(43) Pub. Date: Dec. 30, 2004

(54) Stable immunoprophylactic and therapeutic compositions derived from transgenic plant cells and methods for production

(75) Inventors: Timothy Joe Miller, Lincoln, NE (US); Matthew James Fanton, Lincoln, NE (US); Steven Robert Webb, Westfield, IN (US)

Correspondence Address: SALIWANCHIK LLOYD & SALIWANCHIK, A PROFESSIONAL ASSOCIATION, PO BOX 142950, GAINESVILLE, FL, 32614-2950, US

(73) Assignee: Dow AgroSciences LLC, Indianapolis, IN

(21) Appl. No.: 10/839,509

(22) Filed: May 4, 2004

Related U.S. Application Data
(60) Provisional application No. 60/467,999, filed on May 5, 2003.

(51) Int. Cl. A01H 1/00; C12N 15/82; C12N 7/02

(52) U.S. Cl. 800/288; 435/468

(57) ABSTRACT

The present invention generally relates to the field of immunology and provides immunoprotective compositions and methods for preparing such compositions from transgenic plant cells. The present invention also relates to the field of protein production (e.g., the recombinant production of enzymes, toxins, cell receptors, ligands, signal transducing agents, cytokines, or other proteins expressed in transgenic plant cell culture) and provides compositions Comprising these proteins.

### CROSS REFERENCE TO RELATED APPLICATIONS

This application claims the benefit of U.S. Provisional Application 60/467,999, filed May 5, 2003, which is hereby incorporated by reference in its entirety, including all figures, tables, amino acid sequences and polynucleotide sequences.

### FIELD OF INVENTION

The present invention generally relates to the field of immunology and provides immunoprotective compositions and methods for preparing such compositions from transgenic plant cells. The present invention also relates to the field of protein production (e.g., the recombinant production of enzymes, toxins, cell receptors, ligands, signal transducing agents, cytokines, or other proteins expressed in transgenic plant cell culture) and provides compositions comprising these proteins.

### BACKGROUND OF THE INVENTION

Systemic immunity to a particular pathogen results from activation of the innate or T-cell/B-cell mediated immune system in response to foreign agents. Often, those agents can be antigens of a particular pathogenic organism or a vaccine designed to protect against a particular pathogenic agent. Exposure to pathogens is often through mucosal surfaces that are constantly exposed and challenged by pathogenic organisms.

Mucosal and oral immunity results in the production of sIgA (secretory IgA) antibodies that are secreted by mucosal surfaces of the respiratory tract, gastrointestinal tract, the genitourinary tract and in secretions from all secretory glands. McGhee, J. R. et al., Annals N.Y. Acad. Sci. 409, (1983). These sIgA antibodies act to prevent colonization of pathogens on a mucosal surface (Williams, R. C. et al., Science 177, 697 (1972); McNabb, P. C. et al., Ann. Rev. Microbiol. 35, 477 (1981) and are an important feature of immune defense mechanism for the prevention of colonization or invasion through a mucosal surface. The production of sIgA can be stimulated either by local immunization of the secretory gland or tissue or by presentation of an antigen to either the GALT (gut-associated lymphoid tissue or Peyer's patches) or the BALT (bronchial-associated lymphoid tissue). Cebra, J. J. et al., Cold Spring Harbor Symp. Quant. Biol. 41, 210 (1976); Bienenstock, J. M., Adv. Exp. Med. Biol. 107, 53 (1978); Weisz-Carrington, P. et al., J. Immunol. 123, 1705 (1979); McCaughan, G. et al., Internal Rev. Physiol 28, 131 (1983). Membranous microfold cells, otherwise known as M Cells, cover the surface of the GALT and BALT and may be associated with other secretory mucosal surfaces. M cells act to sample antigens from the luminal space adjacent to the mucosal surface and transfer such antigens to antigen-presenting cells (dendritic cells and macrophages), which in turn present the antigen to a T lymphocyte (in the case of T-dependent antigens). B cells are then stimulated to proliferate, migrate and ultimately be transformed into an antibody-secreting plasma cell producing IgA against the presented antigen. When the antigen is taken up by M cells overlying the GALT and BALT, a generalized mucosal immunity results with sIgA against the antigen being produced by all secretory tissues in the body, Cebra et al., supra; Bienenstock et al., supra; Weinz-Carrington et al., supra; McCaughan et al., supra. Immune protection by oral exposure is therefore an important route to stimulate a generalized mucosal immune response and, in addition, leads to local stimulation of a secretory immune response in the oral cavity and in the gastrointestinal tract.

Moreover, mucosal immunity can be advantageously transferred to offspring. Immunity in neonates may be passively acquired through colostrum and/or milk. This has been referred to as lactogenic immunity and is an efficient way to protect animals during early life. sIgA is the major immunoglobulin in milk and is most efficiently induced by mucosal immunization.

The M cells overlying the Peyer's patches of the gut-associated lymphoid tissue are capable of taking up a diversity of antigenic material and particles (Sneller, M. C. and Strober, W., J. Inf. Dis. 154, 737 (1986). Because of their abilities to take up latex and polystyrene spheres, charcoal, microcapsules and other soluble and particulate matter, it is possible to deliver a diversity of materials to the GALT independent of any specific adhesive-type property of the material to be delivered. Therefore, compositions and means for producing stable and robust particles of appropriates size as, plant-derived immunoprotective antigens would greatly facilitate the development of plant-produced, mucosal vaccines against animal pathogens.

Recombinant DNA technology has provided substantial improvements in the safety, quality, efficacy and cost of pharmaceutical and veterinary medicaments including vaccines. Plant produced mucosal vaccines were invented by Curtiss & Cardineau. See U.S. Pat. Nos. 5,654,184; 5,679,880 and 5,686,079 herein incorporated by reference. Others have described transgenic plants expressing immunoprotective antigens and methods for production including Arntzen, Mason and Lam. See U.S. Pat. Nos. 5,484,717; 5,914,123; 6,034,298; 6,136,320; 6,194,560; and 6,395,964 herein incorporated by reference.

Plant cell production using cell culture in defined media avoids the need for animal-sourced components in growth media essentially eliminating the risk of transmitting pathogenic contaminants from the production process. Plants cells are capable of posttranslational glycosylation, and plant cell growth media is generally less expensive, easier to handle and prepare as compared to conventional growth media presently used in the manufacture of vaccines.

Vaccine antigens and proteins of pharamacological or relevant biological activity produced in plant systems offer a number of advantages over conventional production systems. Plant derived subunit proteins cannot revert to virulence (a feature of live conventionally or recombinant produced live vectored vaccines). Subunit proteins produced from conventional manufacturing methods may be difficult to produce and purify due to protein instability and biochemical extraction issues, and subunit vaccine components that require glycosylation will not be glycosylated when produced in prokaryotes.

Plants provide unique benefits that are difficult to derive from any single conventional or mammalian derived recombinant DNA systems. Traditionally, subunit vaccines or biologically active proteins are: 1) difficult to purify from recombinant or conventional sources because of low yields making their production prohibitive; 2) unstable due to the proteolysis, pH, or solvents used during purification; 3) less efficacious because they are not native, or the purification process denatures key epitopes; and 4) hampered with extraneous materials of biological origin when produced in mammalian systems (mentioned above).

### SUMMARY OF THE INVENTION

The invention is based on the unexpected finding that mechanically or physically disrupted plant cells genetically transformed to express immunogens or other polypeptides produce biologically active proteins and immunoprotective particles useful in vaccine, industrial, pharmaceutical and pharmacological preparations. Furthermore, these proteins display stability and robustness under formulation and downstream processing functions.

The invention provides a method for making stable and efficacious compositions comprising particles prepared from transformed plant cells that express at least one immunoprotective antigen or functional protein which accumulates in the plant cell culture during late exponential and stationary growth. The antigens or functional proteins accumulate in the cytoplasmic cell wall and membrane areas of the plant cell and can be released, in the form of particles, by mechanical or physical disruption or some other means. Furthermore, antigen or functional proteins are stabilized in a biologically active form in the cytoplasmic cell wall and membranes of the plant cell and remain stabile and active during and after the claimed methods. In further embodiments, the methods of antigen or functional protein production include the use of lower plants, monocot or dicot plants, cells and cultures. Further embodiments of the method provide for the production of immunogenic proteins in immunoprotective particles of particular pathogenic viruses including, but not limited to the HA (hemagglutinin) protein of AIV (Avian Influenza Virus), a type 1 glycoprotein; the HN (hemagglutinin/neuraminidase) protein of avian NDV (Newcastle Disease Virus) a type 2 glycoprotein, (See U.S. Pat. No. 5,310,678, herein incorporated by reference); a structural protein, VP2, of infectious bursa disease virus (IBDV); an enzyme ADP ribosyl transferase (LT-A subunit of heat labile toxin of E. coli); a bacterial toxin LT of E. coli made up of two subunits, human viruses including but not limited to picomaviruses such as foot-and-mouth disease virus (FMDV), poliovirus, human rhinovirus (HRV), hepatitis A virus (HAV), immunodeficiency virus (HIV), human papillomavirus (HPV), herpes simplex virus (HSV), and respiratory syncytial virus (RSV). The invention also provides biologically active compositions comprising plant cell soluble extracts, bearing at least one immunoprotective antigen or biologically active protein that accumulate in stationary phase in cytoplasmic cell wall and membrane structures, can easily be extracted with a means such as mechanical disruption, are stable when stored frozen, freeze dried or in suspension, and have features that are similar to native protein. Furthermore, these proteins are deposited in late exponential stage and stationary phase when expressed by several different types of promoter systems including but not limited to the S35 of cauliflower mosaic virus, cassaya vein mosaic virus, monopine/octopine promoter of Agrobacterium tumerfacians. These compositions comprised of recombinant protein and plant cell material can be put in association with one or more pharmaceutically acceptable adjuvants, diluents, carriers, or excipients. In further embodiments, the compositions include lower plants, monocot or dicot-derived particles as well as particles derived from specific plant cells and cultures. Further embodiments of the claimed compositions comprise an enzyme ADP ribosylase; a structural protein VP2; a type 1 glycoprotein; and a type 2 glycoprotein produced in plant cells. Specific immunogenic proteins of certain pathogenic viruses including HN protein of avian NDV and HA protein of AIV are also embodiments of the subject invention.

### BRIEF DESCRIPTION OF THE FIGURES

The file of this patent contains at least one drawing executed in color. Copies of this patent or patent application publication with color drawings will be provided by the Patent and Trademark Office upon request and payment of the necessary fee.

FIGS. 1a and 1b (SEQ ID NOS: 1 and 2) The plant optimized coding sequence and protein sequence of the HN gene of NDV strain “Lasota”.

FIG. 2. Map of pBBV-PHAS-iaaH that contains the plant selectable marker PAT (phosphinothricin acetyl transferase) driven by the constitutive CsVMV (cassaya vein mosaic virus) promoter and terminated by the MAS 3′ (mannopine synthase) element. LB and RB (left and right T-DNA border) elements from Agrobacterium that delineate the boundaries of the DNA that is integrated into the plant genome.

FIG. 3. Map of pC!H which is a “template vector” used as a starting plasmid for a variety of plant expression vectors for expressing immunoprotective antigens.

FIG. 4. Map of pCHN expression vector for NDV HN protein. The HN expression vector or cassette is driven by the constitutive CsVMV promoter and terminated by the soybean vspB 3′ element.

FIG. 5. Map of pgHN expression vector for NDV HN protein. The HN expression cassette is driven by the tuber-specific GBSS promoter with TEV 5′ UTR and terminated by the soybean vspB 3′ element.

FIG. 6. Map of pgHN151 expression vector for NDV HN protein. The HN expression cassette is driven by the tuber-specific GBSS promoter with its native 5′ UTR and intron, and terminated by the soybean vspB 3′ element. The vector is derived from pBBV-PHAS-iaaH, containing the plant selectable marker PAT driven by the CsVMV promoter and terminated by the MAS 3′ element. LB and RB, left and right T-DNA border elements that delineate the boundaries of the DNA that is integrated into the plant genome.

FIG. 7. Map of pgHN153 expression vector for NDV HN protein. The HN expression cassette is driven by the tuber-specific GBSS promoter with its native 5′ UTR and intron, and terminated by the bean phaseolin 3′ element. The vector is derived from pBBV-PHAS-iaaH, containing the plant selectable marker PAT driven by the CsVMV promoter and terminated by the MAS 3′ element. LB and RB, left and right T-DNA border elements that delineate the boundaries of the DNA that is integrated into the plant genome.

FIG. 8. Map of pMHN expression vector for NDV HN protein. The HN expression cassette is driven by the constitutive 4OCSΔMAS promoter (P2 direction) and terminated by the soybean vspB 3′ element. The vector is derived from pBBV-PHAS-iaaH, containing the plant selectable marker PAT driven by the CsVMV promoter and terminated by the MAS 3′ element. LB and RB, left and right T-DNA border elements that delineate the boundaries of the DNA that is integrated into the plant genome.

FIG. 9. Map of pCHA expression vector for the HA gene of the AIV A/turkey/Wisconsin/68 (H₅N₉).

FIG. 10 (SEQ ID NOS: 3 and 4). The DNA and protein sequences of the HA gene of AIV A/turkey/Wisconsin/68 (H₅N₉).

FIG. 11. Map of pGLTB intermediate vector.

FIG. 12. Map of pCLT105 intermediate vector.

FIG. 13. pDAB2423. Binary vector encoding VP2.

FIG. 14 (SEQ ID NO: 10). The DNA sequence of VP2 gene of IBDV Infectious Bursal Disease (IBD) virus, very virulent strain Ehime 91.

FIGS. 15-18. Production, growth and accumulation of expressed protein for CHN-18, CHA-13, SLT102, and CVP2-002. FIG. 15. Results of growth for CHA-13 in 10 liter fermentor. The closed squares: growth of the Cn-18 NT-1 transgenic cell using packed cell volume (PCV) from a 10 ml sample at various times after inoculation. Closed triangles: accumulation of HN protein (per 10 liter fermentor run) using a quantitative ELISA assay described in Example 7. The closed diamonds are the accumulation of hemagglutination titer described in Example 8, the hemagglutination titer observed at day 1 is from the inoculum taken from a 13 day (stationary) shaker culture. FIG. 16. Results of growth for CHA-13 in 10 liter fermentor. The closed squares are the growth of the CHA-13 NT-1 transgenic cell using packed cell volume (PCV) from a 10 ml sample at various times after inoculation. The open triangles show the sucrose concentration, sucrose is used as the carbon source it is rapidly converted to dextrose (open squares) and can no longer be detected 48 hours after inoculation into the culture. The accumulation of the HA protein by quantitative ELISA is represented by the closed triangles from the cell extract of CHA-13 NT-1. FIG. 17. Results of growth for CVP2-002 in 10 liter fermentor. The closed diamonds depict the growth of the CVP2-002 NT-1 transgenic cell using packed cell volume (PCV) from a 10 ml sample at various times after inoculation. The open triangles is the sucrose concentration, sucrose is used as the carbon source it is rapidly converted to dextrose (*) and can no longer be detected 48 hours after inoculation into the culture. The accumulation of the VP2 protein by quantitative ELISA is represented by the closed triangles from the cell extract of CVP2-002 NT-1. FIG. 18. Accumulation of LT in shaker flask cultures, concentration was determined by LT quantitative ELISA in Example 7. Growth curve was not determined in this study but PCV for SLT-102 NT-1 cell exhibit the same growth and production as seen for NT-1 transgenic cell lines in FIGS. 15-17.

FIG. 19. Stable production of protein from transgenic cell line CHN-18. Stability of the quantitative ELISA signal from samples prepared from CHN-18 NT-1. Supertantants from CHN-18 were isolated by harvesting cells from 10 liter fermentor as described in Example 3. After the a single microfluidization to disrupt the cells the samples were then filtered through either 0.45 micron or 0.2 micron filters and stored at 25° C.

FIGS. 20 and 21. Confocal scanning microscopy. FIG. 20. MHN-41 stained cells. Green: Cells stained with Cy2 dye labeled for Rb anti-HN Polyclonal antibody (upper left). Blue: Cells stained with the Cy5 dye labeled for 4A ascites fluid.(lower left). Red: Propidium Iodide stained nucleus (upper right). Light Blue/Red. Digital merged image of green, blue and red images. No staining with either antibody is observed in the nucleus of the cells. Because of the intensely stained areas along the entire cell wall and membrane of the intracellular cytoplasm, the vacuole cannot be distinquished.

FIG. 21. Control NT-1 cells. Staining of control NT-1 cells, panels to the left are cells stained with either the Rb anti-HN polyclonal or 4A ascites fluid; the right panels are propidium iodide stained NT-1 cells.

FIGS. 22 and 23. Electron micrographs illustrating the localization of transgenically produced polypeptide. FIG. 22. Electron microscopy of osmium tetraoxide fixed cells from NT-1 control cells, CHN-18 transgenic cells and MHN-41 transgenic cells. The magnification of each frame is indicated, control cells at 16,000 magnification, CHN-18 at 50,000 magnification, and MHN-41 at 26,000 magnification. FIG. 23. Immunogold staining for electron microscopy of NT-1 control cells, CHN-18 transgenic cells and MN-41 transgenic cells.

FIGS. 24-31. Maps of binary, intermediate and expression vectors. FIG. 24: Basic binary vector (BBV) map. FIG. 25: Intermediate pDAB2407 map. FIG. 26: Synthesized VP2 in Bluescript vector, provided by PICOSCRIPT (Houston, Tex.). FIG. 27: Map of intermediate vector, pDAB2406. FIG. 28: Map of intermediate vector, pDAB2415. FIG. 29: Map of intermediate vector, pDAB2418. FIG. 30: Map of intermediate vector, pDAB2416. FIG. 31: Dicot expression vector pDAB2423 map illustrating VP2 driven by the CsVMV promoter, terminated by Atu ORF24 3′UTR, with an upstream RB7 MAR element. The selectable marker, PAT, is regulated by At Ubi 10 promoter and Atu ORF1 3′ UTR.

### SUMMARY OF THE SEQUENCES

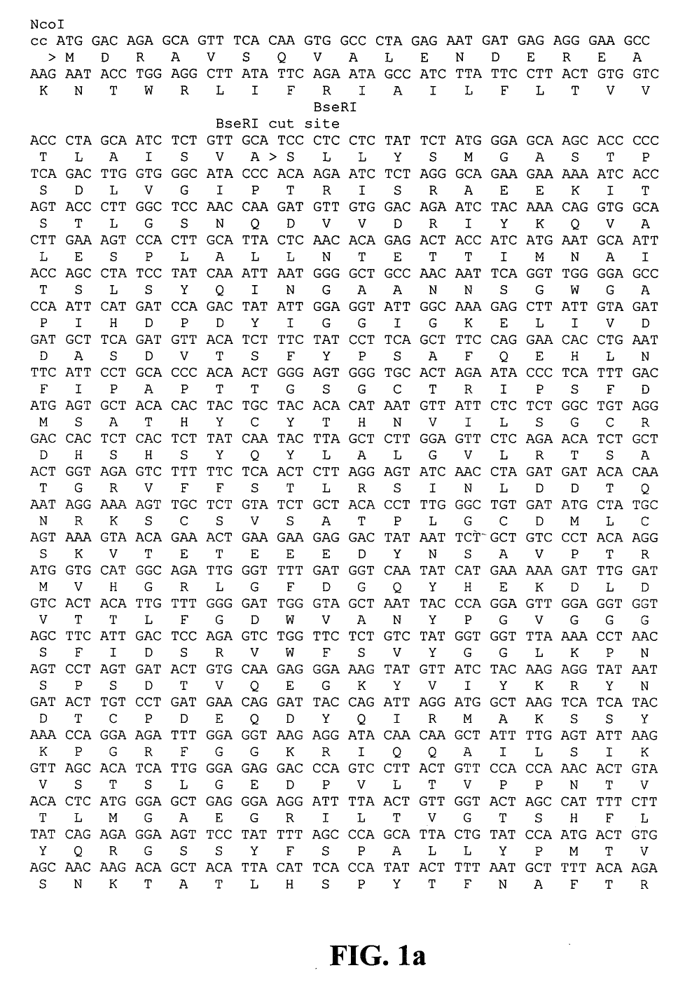

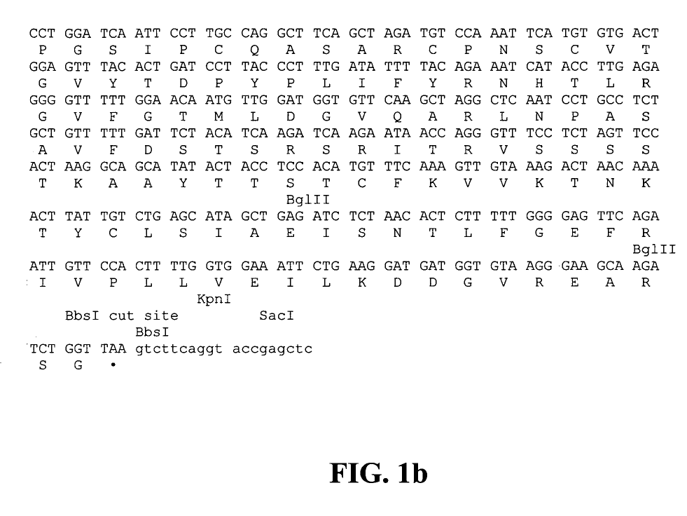

SEQ ID NOS: 1 and 2, shown in FIGS. 1a and 1b, are the plant optimized coding sequence and protein sequence of the HN gene of NDV strain “Lasota”.

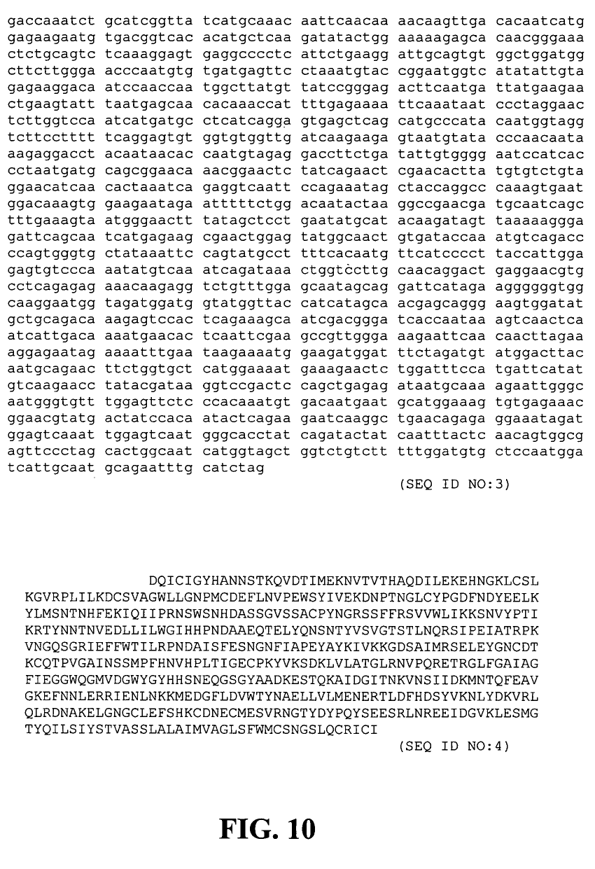

SEQ ID NOS: 3 and 4, shown in FIG. 10, are the DNA and protein sequences of the HA gene of AIV A/turkey/Wisconsin/68 (H₅N₉).

SEQ ID NO:5 is a PCR primer used to end-tailor the CsVMV promoter on pCP!H.

SEQ ID NO:6 is a PCR primer used to end-tailor the CsVMV promoter on pCP!H.

SEQ ID NO:7 is a mutagenic primer used to create a Nco I site.

SEQ ID NO:8 is forward primer complimentary to the 5′ region.

SEQ ID NO:9 is a mutagenic primer used to create a XhoI I site.

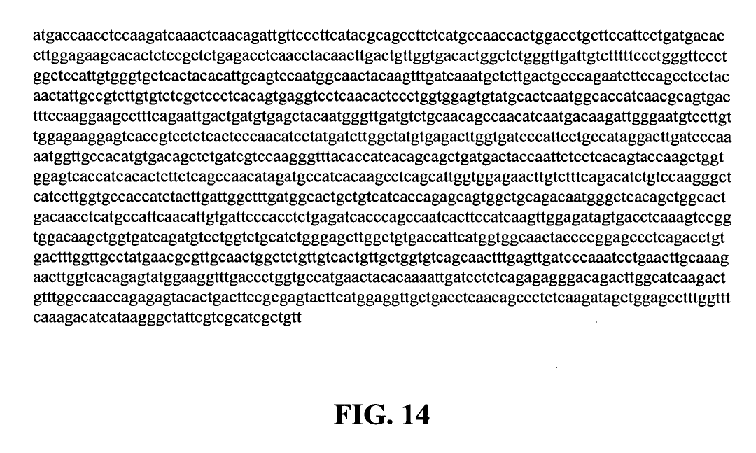

SEQ ID NO:10 shown in FIG. 14 is the DNA sequence of VP2 gene of infectious bursal disease virus.

SEQ ID NO: 11 is a plant-optimized DNA sequence encoding a variation of E/91 VP2 (1425 bases). The coding region for E/91 plant-optimized VP2 comprises bases 16 to 1383 (1371 bases). Six frame stops are found at bases 1384 to 1425.

SEQ ID NO: 12 comprises the sequence of the E/91 VP2 protein encoded by the plant-optimized version of the E/91 VP2 coding region (SEQ ID No. 11).

SEQ ID NO: 13 is the DNA sequence encoding translation termination (“Stop”) codons in six reading frames. The sequence was used to terminate translation of inadvertant open reading frames following DNA integration during transformation and includes Sac I BstE II, and Bgl II restriction enzyme recognition sites (Tsukamoto K., Kojima, C., Komori, Y., Tanimura, N., Mase, M., and Yamaguchi, S. (1999) Protection of chickens against very virulent infectious bursal disease virus (IBDV) and Marek's disease virus (MDV) with a recombinant MDV expressing IBDV VP2. Virol. 257: 352-362.)

### DETAILED DESCRIPTION OF THE INVENTION

An immunogen or immunoprotective antigen is a substance that elicits an innate, humoral and/or cellular immune response in healthy animals such that the animal is protected against future exposure to a pathogen bearing the immunogen. These pathogens are typically agents such as viruses, bacteria, fungi and protozoa. Immunogens may also be antigenic portions of pathogens including cell wall components and viral coat proteins.

Biologically active proteins include, but are not limited to enzymes, toxins, cell receptors, ligands, signal transducing agents, cytokines, or other proteins expressed in transgenic plant cell culture; including, carbohydrases (e.g., alpha-amylase [bacterial α-amylase (e.g., Bacillus subtilis), fungal α-amylase (e.g., Aspergillus niger), alkaline α-amylase]; β-amylase; cellulase; β-glucanase; exo-β-1,4-glucanase, endo-β-1,4-glucanase; β-glucosidase; dextranase; dextrinase; α-galactosidase (melibiase); glucoamylase; hemmicellulase/pentosanase/xylanase; invertase; lactase; naringinase; pectinase; pullulanase); proteases (e.g., acid proteinase; alkaline protease; bromelain; pepsin; aminopeptidase; endo-peptidase; subtilisin); lipases and esterases (e.g., phospholidases; pregastric esterases; phosphatases; aminoacylase; glutaminase; lysozyme; penicillin acylase; isomerase); oxireductases (e.g., alcohol dehydrogenase; amino acid oxidase; catalase; chloroperoxidase; peroxidase); lyases (e.g., acetolactate decarboxylase; aspartic β-decarboxylase; histidase); or transferases (e.g., cyclodextrin glycosyltransferase). Polynucleotide sequences encoding these enzymes (or toxins, cell receptors, ligands, signal transducing agents, or cytokines suitable for expression in the expression systems of the subject invention) can be obtained from commercial databases such as EMBL, SWISSPROT, or the NCBI database. Typically biologically active proteins produced in transgenic plant cell cultures are equivalent in functional or structural activity to the same proteins isolated from natural sources.

A biologically active protein particle is defined as a heterogeneous particle or aggregate composed of the recombinant protein, plant proteins, lipid, carbohydrate, nucleic acid or combinations thereof derived from a transgenic plant cell expressing a biologically active protein that is prepared by the methods of the present invention. In certain embodiments, the biologically active protein particle can be part of, or associated with, lipid vesicles, membrane fragments, cell wall fragments, subcellular organelles or fragments, or storage proteins that is typically derived from late exponential and stationary growth phase of the transformed plant cell. The claimed particles are highly stabile and maintain the recombinant protein in a highly stabile and biologically active conformation. In other embodiments the particle is defined as the material that can easily be suspended in buffer or culture supernatant by mechanically or physically disrupting a late exponential or stationary growth transgenic cell culture expressing a protein from a recombinant gene introduced into the plant cell.

An immunoprotective particle is derived or obtained from a transgenic plant cell that has been genetically engineered to express an immunoprotective antigen. The claimed immunoprotective particle is a heterogeneous particle or aggregate composed of the recombinant immunoprotective antigen, protein, lipid, carbohydrate, nucleic acid or combinations thereof derived from the engineered transgenic plant cell that, when appropriately administered to an animal, including humans, provides protection against future exposure to a pathogen bearing the immunogen. The claimed particles are highly stabile and maintain the recombinant immunoprotective antigen in a highly stabile and biologically active conformation. The immunoprotective particle is obtained by mechanical or physical disruption of the engineered cell followed by separating the cellular debris from the immunoprotective particle. In certain embodiments, the particle can be part of, or associated with, lipid vesicles, membrane fragments, cell wall fragments, subcellular organelles or fragments, or storage proteins that is derived from late exponential and stationary growth phase of the transformed plant cell. In other embodiments the particle is defined as the material that can easily be suspended in buffer or culture supernatant by mechanically or physically disrupting a late exponential or stationary growth transgenic cell culture expressing a protein from a recombinant gene introduced into the plant cell.

Lower plant is defined as any non-flowering plant including ferns, gymnosperms, conifers, horsetails, club mosses, liver warts, hornwarts, mosses, red algaes, brown algaes, gametophytes, sporophytes of pteridophytes, and green algaes; especially preferred are mosses.

Vaccination and vaccinating is defined as a means for providing protection against a pathogen by inoculating a host with an immunogenic preparation, an immunoprotective particle, or an immunogenic preparation of a pathogenic agent, or a non-virulent form or part thereof, such that the host immune system is stimulated and prevents or attenuates subsequent unwanted pathology associated with the host reactions to subsequent exposures of the pathogen.

A vaccine is a composition used to vaccinate an animal, including a human, that contains at least one immunoprotective antigenic substances.

A pathogenic organism is a bacterium, virus, fungus, or protozoan that causes a disease or induced/controlled physiologic condition in an animal that it has infected.

For purposes of this specification, an adjuvant is a substance that accentuates, increases, moderates or enhances the immune response to an immunogen or antigen. Adjuvants typically enhance both the humor and cellular immune response but an increased response to either in the absence of the other qualifies to define an adjuvant. Moreover, adjuvants and their uses are well known to immunologists and are typically employed to enhance the immune response when doses of immunogen are limited, when the immunogen is poorly immunogenic, or when the route of administration is sub-optimal. Thus the term ‘adjuvating amount’ is that quantity of adjuvant capable of enhancing the immune response to a given immunogen or antigen. The mass that equals an ‘adjuvating amount’ will vary and is dependant on a variety of factors including, but not limited to, the characteristics of the immunogen, the quantity of immunogen administered, the host species, the route of administration, and the protocol for administering the immunogen. The ‘adjuvating amount’ can readily be quantified by routine experimentation given a particular set of circumstances. This is well within the ordinarily skilled artisan's purview and typically employs the use of routine dose response determinations to varying amounts of administered immunogen and adjuvant. Responses are measured by determining serum antibody titers or cell-mediated responses raised to the immunogen using enzyme linked immunosorbant assays, radio immune assays, hemagglutination assays and the like.

The present invention also provides pharmaceutical and veterinary compositions comprising an immunoprotective or biologically active protein or particle or composition of the present invention in combination with one or more pharmaceutically acceptable adjuvants, carriers, diluents, and excipients. Such pharmaceutical compositions may also be referred to as vaccines and are formulated in a manner well known in the pharmaceutical and vaccine arts.

Administering or administer is defined as the introduction of a substance into the body of an animal, including a human, and includes oral, nasal, ocular, rectal, vaginal and parenteral routes. The claimed compositions may be administered individually or in combination with other therapeutic agents via any route of administration, including but not limited to subcutaneous (SQ), intramuscular (IM), intravenous (IV), intraperitoneal (IP), intradermal (ID), via the nasal, ocular or oral mucosa (IN), or orally. Especially preferred is the mucosal route, and most preferred is the oral route.

An effective dosage is an amount necessary to induce an immune response in a human or animal sufficient for the human or animal to effectively resist a challenge mounted by pathogenic agent or to respond to a physiological requirement of the animal such as an autoimmune antigen to diabetes. The dosages administered to such human or animal will be determined by a physician, veterinarian, or trained scientist in the light of the relevant circumstances including the particular immunoprotective particle or combination of particles, the condition of the human or animal, and the chosen route of administration. Generally, effective dosages range from about 1 ng to about 0.5 mg, and preferably from about 1 ug to about 50 ug. For Newcastle Disease Virus (HN antigen) in poultry effective dosages range from about 0.5 ug to about 50 ug, preferably from about 2.5 ug to about 5 ug via the SQ route. Via the IN/ocular mucosal route effective dosages for HN in poultry range from about 0.5 ug to about 50 ug, preferably from about 5 ug to about 25 ug, and more preferably from about 10 ug to about 12 ug. For Avian Influenza Virus (HA antigen) effective dosages range from about 0.5 ug to about 50 ug, preferably from about 1 ug to about 30 ug, and more preferably from about 24 ug to about 26 ug via the IN/ocular route and preferably from about 1 ug to about 5 ug via the SQ route. For Infectious Bursal Disease (VP2 antigen) in poultry effective dosages range from 0.5 ug to about 50 ug, preferably from about 5 ug to about 25 ug, and more preferably from about 5 ug to about 20 ug via the SQ route. For LT antigen effective oral dosages range from about 50 ng to about 250 ng, preferably from about 100 ng to about 200 ng. For LT antigen effective SQ or IN/ocular dosages range from about 50 ng to about 100 ug; preferably from about 1 ug to about 25 ug and more preferably from about 2 ug to about 10 ug. The dosage ranges presented herein are not intended to limit the scope of the invention in any way and are presented as general guidance for the skilled practitioner.

Bird is herein defined as any warm-blooded vertebrate member of the class Aves typically having forelimbs modified into wings, scaly legs, a beak, and bearing young in hard-shelled eggs. For purposes of this specification, preferred groups of birds are domesticated chickens, turkeys, ostriches, ducks, geese, and cornish game hens. A more preferred group is domesticated chickens and turkeys. The most preferred bird for purposes of this invention is the domesticated chicken, including broilers and layers (poultry).

The methods and compositions of the present invention are directed toward immunizing and protecting animals, including humans, preferably domestic animals, such as birds (poultry), cows, sheep, goats, pigs, horses, cats, dogs and llamas, and most preferably birds. Certain of these animal species can have multiple stomachs and digestive enzymes specific for the decomposition of plant matter, and may otherwise readily inactivate other types of oral vaccines. While not meant to be a limitation of the invention, ingestion of transgenic plant cells, and compositions derived therefrom, can result in immunization of the animals at the site of the oral mucosa including the tonsils.

For purposes of the present invention the term membrane sequence contemplates that which the ordinarily skilled artisan understands about the term. Membrane anchor sequences include transmembrane protein sequences and are found in many naturally occurring proteins. Such membrane anchor sequences vary in size but always are comprised of a series of amino acids having lipophilic or aliphatic side chains that favor the hydrophobic environment within the membrane. During RNA translation and post translational processing, the anchor sequences integrate and become embedded in the cell membrane and function to anchor, or loosely attach the protein to a cellular membrane component allowing hydrophilic portions of the protein to be exposed to, and interact with, the aqueous milieu inside or outside of the cell.

Storage inclusion body or storage protein herein is defined as proteins the plant uses for nitrogen sources, these proteins are stored during non-productive phases of the plant life cycle (e.g., during stationary phase) and are quickly utilized as sources of energy and nitrogen when the cell is induced into active growth.

Transgenic plant is herein defined as a plant cell culture, plant cell line, plant tissue culture, lower plant, monocot plant, dicot plant, or progeny thereof derived from a transformed plant cell or protoplast, wherein the genome of the transformed plant contains foreign DNA, introduced by laboratory techniques, not originally present in a native, non-transgenic plant cell of the same species. The terms “transgenic plant” and “transformed plant” have sometimes been used in the art as synonymous terms to define a plant whose DNA contains an exogenous DNA molecule.

Construction of gene cassettes for expressing immunoprotective antigens in plants is readily accomplished utilizing well known methods, such as those disclosed in Sambrook et al. (1989); and Ausubel et al., (1987) Current Protocols in Molecular Biology, John Wiley and Sons, New York, N.Y. The present invention also includes DNA sequences having substantial sequence homology with the disclosed sequences encoding immunoprotective antigens such that they are able to have the disclosed effect on expression. As used in the present application, the term “substantial sequence homology” is used to indicate that a nucleotide sequence (in the case of DNA or RNA) or an amino acid sequence (in the case of a protein or polypeptide) exhibits substantial, functional or structural equivalence with another nucleotide or amino acid sequence. Any functional or structural differences between sequences having substantial sequence homology will be de minimis; that is they will not affect the ability of the sequence to function as indicated in the present application. Sequences that have substantial sequence homology with the sequences disclosed herein are usually variants of the disclosed sequence, such as mutations, but may also be synthetic sequences.

In most cases, sequences having 95% homology to the sequences specifically disclosed herein will function as equivalents, and in many cases considerably less homology, for example 75% or 80%, will be acceptable. Locating the parts of these sequences that are not critical may be time consuming, but is routine and well within the skill in the art. Exemplary techniques for modifying oligonucleotide sequences include using polynucleotide-mediated, site-directed mutagenesis. See Zoller et al. (1984); Higuchi et al. (1988); Ho et al. (1989); Horton et al. (1989); and PCR Technology: Principles and Applications for DNA Amplification, (ed.) Erlich (1989).

In most cases mammalian cells, bacterial cells, or other host vector systems used for production of proteins via recombinant DNA do not establish protein stores that can be reused when placed in renewed culture environments. Inclusions bodies described for E. coli, or crystalline proteins of baculovirus, granulosis virus or Bacillus thurengiensis are deposited by the host system for various biological purposes. However, none have been shown to put proteins into storage compartments that can be used by the plant as a nitrogen source during re-cultivation or activation of new growth from resting or stationary phase.

The placement of protein into storage compartments or stable sites in the cell at late stages of stationary phase of NT-1 growth was not an expected feature of expression of proteins in transgenic plant cells cultivated in vitro. Electron microscopy shows dark centers in the leucoplasts and immunogold labeled attachment to proteins in cytoplasmic compartments next to cell wall and membranes (see Example 16). Furthermore, the ability of the NT-1 system to deposit protein into stable compartments regardless of the types of protein expressed or transcriptional promoter system used is an unprecedented observation. Proteins that have been successfully expressed include several different classes of proteins: 1) an enzyme ADP ribosyl transferase, the LTA component of LT enterotoxin of E. coli; 2) fully formed and functional LT holotoxin containing both LTA and LTB subunits derived from E. coli; 3) a structural protein VP2 of infectious bursa disease virus (IBDV); 4) a type 1 viral glycoprotein hemagglutinin (HA) of avian influenza virus (AIV); and, 5) type 2 viral glycoprotein of Newcastle disease virus. In each case the biological activity of the expressed protein was found to be as potent if not more potent than the native protein derived from each respective pathogen. The efficacy associated with the each protein is an unexpected feature for a single type of host cell used for expression of a foreign protein. Another unexpected feature of the stored foreign protein is that it is stable, and as described above, the protein can easily be isolated (for example, by mechanical disruption). The suspended protein or protein-bearing particles can then be freeze dried, frozen, emulsified, homogenized, microfluidized, without loss of signal or stability. Protein and particles of the present invention held in liquid form at 2-7° C. for several months display long half lives; without any stabilizers added, extracts produced by simple mechanical agitation have resulted in preparations with projected half-life of 1-2 years for HN protein of NDV and 13-15 months for LT of E. coli. The proteins produced in accordance with the subject invention are extremely robust and are amenable to various types of formulations that can augment immune response.

Physical or mechanical cell disruption techniques consistent with the claimed methods include but are not limited to conventional cell disruption means such as sonication, microfluidization or other shear-type methods, high shear rotor/stator methods, French press or other pressure methods, and homogenization techniques. Early research and development activities showed that high pressure disruption energies were necessary for extracting HN protein from harvested cells in the form of immunoprotective particles. While sonic disruption was utilized to release HN immunoprotective particles from small fermentor assay volumes (1-10 ml), it was shown to be less effective (>35%) for recovery of HN immunoprotective particles from harvested cell volumes exceeding 1 L, and not amenable to scale-up.

Power titration studies, using a fixed orifice pressure disrupter from Microfludics, Inc., showed that disruption pressure was proportional to the yield of HN immunoprotective particles recovered from NT1-CHN-18 cells. The highest recovery of HN particles was achieved at the maximum pressure setting for the instrument, which was 18,000 psig. Even at the maximum pressure, more than 40% of the total HN protein was present in the discarded, cell debris fraction. Pressure titration curves for this latter experiment suggest that higher lysis pressures may significantly reduce the amount of HN protein in the discarded, cell debris fraction.

The Microfludics product line is considered to be ‘second generation’ in constant cell disruption technology. Microfludics instruments achieve cell disruption by forcing suspended cells through a fixed 0.1 mm turbulent (‘Y’ geometry) orifice, which is attached to reservoir that is emptied at a high flow rate with hydraulic ram. The ram upstroke opens a check valve that fills the reservoir for the next cycle. Microfludics Inc., claims equivalence in scale from roughly 10 ml*min⁻¹ to 10,000 L*hr⁻¹. Cell lysis is thought to be the result of (1) acceleration through the orifice (implosion), (2) pressure differential between the orifice tip and ejection chamber that causes cellular rupture, and/or (3) de-acceleration into the ejection chamber target. Cellular ultrastructure (i.e., cell wall), cell concentration, disruption energy (psig), and the lysis buffer composition are considered the most important variables that influence lysis efficiency.

Earlier first generation instruments were produced by Aminco Inc., as continuous French press cells. These are similar to the Microfludics instruments, except that the orifice diameter and hydraulic pressure are controlled manually. These latter instruments are primarily used for research and development activities for sample volumes less than 50 ml. Third generation constant cell disruption instruments (DeBEE, Inc., and Constant Systems, Inc.) have included improvements such as higher operating pressures (up to 60,000 psig), dual sample chambers to reduce pressure fluctuations, and sample ejection chambers that are operated under vacuum. These improvements have reportedly improved lysis efficiency over first and second generation instruments.

The clarification step of the claimed method includes any separation techniques including but are not limited to gravity sedimentation, centrifugation, floatation, filtration including tangential flow and conventional, and chromatographic techniques including all forms of column and HPLC methods. Preferred methods are low speed centrifugations ranging from about 1000 g to about 5000 g for periods of several minutes.

In preparing the constructs of this invention, the various DNA fragments may be manipulated, so as to provide for the DNA sequences in the proper orientation and, as appropriate, in the proper reading frame. Adapters or linkers may be employed for joining the DNA fragments or other manipulations may be involved to provide for convenient restriction sites, removal of superfluous DNA, removal of restriction sites, or the like.

In carrying out the various steps, cloning is employed, so as to amplify a vector containing the promoter/gene of interest for subsequent introduction into the desired host cells. A wide variety of cloning vectors are available, where the cloning vector includes a replication system functional in E. coli and a marker which allows for selection of the transformed cells. Illustrative vectors include pBR322, pUC series, pACYC184, Bluescript series (Stratagene) etc. Thus, the sequence may be inserted into the vector at an appropriate restriction site(s), the resulting plasmid used to transform the E. coli host (e.g., E. coli strains HB101, JM101 and DH5α), the E. coli grown in an appropriate nutrient medium and the cells harvested and lysed and the plasmid recovered. Analysis may involve sequence analysis, restriction analysis, electrophoresis, or the like. After each manipulation the DNA sequence to be used in the final construct may be restricted and joined to the next sequence, where each of the partial constructs may be cloned in the same or different plasmids.

Vectors are available or can be readily prepared for transformation of plant cells. In general, plasmid or viral vectors should contain all the DNA control sequences necessary for both maintenance and expression of a heterologous DNA sequence in a given host. Such control sequences generally include a leader sequence and a DNA sequence coding for translation start-signal codon, a translation terminator codon, and a DNA sequence coding for a 3′ UTR signal controlling messenger RNA processing. Selection of appropriate elements to optimize expression in any particular species is a matter of ordinary skill in the art utilizing the teachings of this disclosure. Finally, the vectors should desirably have a marker gene that is capable of providing a phenotypical property which allows for identification of host cells containing the vector.

The activity of the foreign coding sequence inserted into plant cells is dependent upon the influence of endogenous plant DNA adjacent the insert. Generally, the insertion of heterologous genes appears to be random using any transformation technique; however, technology currently exists for producing plants with site specific recombination of DNA into plant cells (see WO 91/09957). Any method or combination of methods resulting in the expression of the desired sequence or sequences under the control of the promoter is acceptable.

The present invention is not limited to any particular method for transforming plant cells. Technology for introducing DNA into plant cells is well-known to those of skill in the art. Four basic methods for delivering foreign DNA into plant cells have been described. Chemical methods (Graham and van der Eb, Virology, 54(O₂):536-539, 1973; Zatloukal, Wagner, Cotten, Phillips, Plank, Steinlein, Curiel, Bimstiel, Ann. N.Y. Acad. Sci., 660:136-153, 1992); Physical methods including microinjection (Capecchi, Cell, 22(2):479-488, 1980), electroporation (Wong and Neumann, Biochim. Biophys. Res. Commun. 107(2):584-587, 1982; Fromm, Taylor, Walbot, Proc. Natl. Acad. Sci. USA, 82(17):5824-5828,1985; U.S. Pat. No. 5,384,253) and the gene gun (Johnston and Tang, Methods Cell. Biol., 43(A):353-365, 1994; Fynan, Webster, Fuller, Haynes, Santoro, Robinson, Proc. Natl. Acad. Sci. USA 90(24):11478-11482, 1993); Viral methods (Clapp, Clin. Perinatol., 20(1):155-168, 1993; Lu, Xiao, Clapp, Li, Broxmeyer, J. Exp. Med. 178(6):2089-2096, 1993; Eglitis and Anderson, Biotechniques, 6(7):608-614, 1988; Eglitis, Kantoff, Kohn, Karson, Moen, Lothrop, Blaese, Anderson, Avd. Exp. Med. Biol., 241:19-27, 1988); and Receptor-mediated methods (Curiel, Agarwal, Wagner, Cotten, Proc. Natl. Acad. Sci. USA, 88(19):8850-8854, 1991; Curiel, Wagner, Cotten, Birnstiel, Agarwal, Li, Loechel, Hu, Hum. Gen. Ther., 3(2):147-154, 1992; Wagner et al., Proc. Natl. Acad. Sci. USA, 89 (13):6099-6103, 1992).

The introduction of DNA into plant cells by means of electroporation is well-known to those of skill in the art. Plant cell wall-degrading enzymes, such as pectin-degrading enzymes, are used to render the recipient cells more susceptible to transformation by electroporation than untreated cells. To effect transformation by electroporation one may employ either friable tissues such as a suspension culture of cells, or embryogenic callus, or immature embryos or other organized tissues directly. It is generally necessary to partially degrade the cell walls of the target plant material to pectin-degrading enzymes or mechanically wounding in a controlled manner. Such treated plant material is ready to receive foreign DNA by electroporation.

Another method for delivering foreign transforming DNA to plant cells is by microprojectile bombardment. In this method, microparticles are coated with foreign DNA and delivered into cells by a propelling force. Such micro particles are typically made of tungsten, gold, platinum, and similar metals. An advantage of microprojectile bombardment is that neither the isolation of protoplasts (Cristou et al., 1988, Plant Physiol., 87:671-674,) nor the susceptibility to Agrobacterium infection is required. An illustrative embodiment of a method for delivering DNA into maize cells by acceleration is a Biolistics Particle Delivery System, which can be used to propel particles coated with DNA or cells through a screen onto a filter surface covered with corn cells cultured in suspension. The screen disperses the particles so that they are not delivered to the recipient cells in large aggregates. For the bombardment, cells in suspension are preferably concentrated on filters or solid culture medium. Alternatively, immature embryos or other target cells may be arranged on solid culture medium. The cells to be bombarded are positioned at an appropriate distance below the macroprojectile stopping plate. In bombardment transformation, one may optimize the prebombardment culturing conditions and the bombardment parameters to yield the maximum numbers of stable transformants. Both the physical and biological parameters for bombardment are important in this technology. Physical factors are those that involve manipulating the DNA/microprojectile precipitate or those that affect the flight and velocity of either the microprojectiles. Biological factors include all steps involved in manipulation of cells before and immediately after bombardment, the osmotic adjustment of target cells to help alleviate the trauma associated with bombardment, and also the nature of the transforming DNA, such as linearized DNA or intact supercoiled plasmids.

Agrobacterium-mediated transfer is a widely applicable system for introducing foreign DNA into plant cells because the DNA can be introduced into whole plant tissues, eliminating the need to regenerate an intact plant from a protoplast. The use of Agrobacterium-mediated plant integrating vectors to introduce DNA into plant cells is well known in the art. See, for example, the methods described in Fraley et al., 1985, Biotechnology, 3:629; Rogers et al., 1987, Meth. in Enzymol., 153:253-277. Further, the integration of the Ti-DNA is a relatively precise process resulting in few rearrangements. The region of DNA to be transferred is defined by the border sequences, and intervening DNA is usually inserted into the plant genome as described in Spielmann et al., 1986, Mol. Gen. Genet., 205:34; Jorgensen et al., 1987, Mol. Gen. Genet., 207:471.

Modern Agrobacterium transformation vectors are capable of replication in E. coli as well as Agrobacterium, allowing for convenient manipulations. Moreover, recent technological advances in vectors for Agrobacterium-mediated gene transfer have improved the arrangement of genes and restriction sites in the vectors to facilitate construction of vectors capable of expressing various proteins or polypeptides. Convenient multi-linker regions flanked by a promoter and a polyadenylation site for direct expression of inserted polypeptide coding genes are suitable for present purposes. In addition, Agrobacterium containing both armed and disarmed Ti genes can be used for the transformations.

Transformation of plant protoplasts can be achieved using methods based on calcium phosphate precipitation, polyethylene glycol treatment, electroporation, and combinations of these treatments (see, e.g., Potrykus et al., 1985, Mol. Gen. Genet., 199:183; Marcotte et al., Nature, 335:454, 1988). Application of these systems to different plant species depends on the ability to regenerate the particular species from protoplasts.

Once the plant cells have been transformed, selected and checked for antigen expression, it is possible in some cases to regenerate whole fertile plants. This will greatly depend on the plant species chosen. Methods for regenerating numerous plant species have been reported in the literature and are well known to the skilled artisan. For practice of the present invention, it is preferable to transform plant cell lines that can be cultured and scaled-up rapidly by avoiding the generally lengthy regeneration step. In addition, the use of plant cell cultures avoids open field production and greatly reduces the chances of gene escape and food contamination. Tobacco suspension cell cultures such NT-1 and BY-2 (An, G., 1985 Plant Physiol. 79, 568-570) are preferred because these lines are particularly susceptible to handling in culture, are readily transformed, produce stably integrated events and are amenable to cryopreservation.

The tobacco suspension cell line, NT-1, is suitable for the practice of the present invention. NT-1 cells were originally developed from Nicotiana tabacum L.cv. bright yellow 2. The NT-1 cell line is widely used and readily available; though, any tobacco suspension cell line is consistent with the practice of the invention. It is worth noting that the origins of the NT-1 cell line are unclear. Moreover, the cell line appears variable and is prone to change in response to culture conditions. NT-1 cells suitable for use in the examples below are available from the American Type Culture Collection under accession number ATCC No. 74840. See also U.S. Pat. No. 6,140,075, herein incorporated by reference in its entirety.

Many plant cell culture techniques and systems ranging from laboratory-scale shaker flasks to multi-thousand liter bioreactor vessels have been described and are well know in the art of plant cell culture. See for example Fischer, R. et al, 1999 Biotechnol. Appl. Biochem. 30, 109-112 and Doran, P., 2000 Current Opionions in Biotechnology 11, 199-204. After the transformed plant cells have been cultured to the mass desired, they are harvested, gently washed and placed in a suitable buffer for disruption. Many different buffers are compatible with the present invention. In general the buffer is an aqueous isotonic buffered salt solution at or near a neutral pH value that does not contain harsh detergents that can be used to solubilze membranes. Preferred buffers include Dulbecco's Phosphate Buffered Saline and PBS containing 1 mM EDTA.

In one embodiment, cells can be disrupted by sonication. The washed cells are placed in buffer in a range of about 0.01 gm/ml to about 5.0 gm/ml, preferably in a range of about 0.1 gm/ml to about 0.5 gm/ml (washed wet weight cells per volume of buffer). Many commercially available sonication instruments are consistent with the invention and sonication times range from about 5 to about 20 seconds, preferably about 15 to about 20 seconds. The resulting may range in size from a few microns to several hundred microns and expose the recombinant immunoprotective proteins or other biologically active proteins.

### EXAMPLE 1

### Vectors

Gene Construction: The coding sequence of the HN gene of NDV strain “Lasota” (Genbank accession AF077761), HA gene of AIV strain ATurkey/Wisconsin/68, VP2 gene of IBDV stain E19 (GenBank accession number X00493), and LT gene of E. coli were analyzed for codon use and the presence of undesired sequence motifs that could mediate spurious mRNA processing and instability, or methylation of genomic DNA. See Adang M J, Brody M S, Cardineau G, Eagan N, Roush R T, Shewmaker C K, Jones A, Oakes J V, McBride K E (1993) The construction and expression of Bacillus thuringiensis cryIIIA gene in protoplasts and potato plants. Plant Mol Biol 21:1131-1145. A plant-optimized coding sequence was designed with hybrid codon preference reflecting tomato and potato codon usage (Ausubel F., et al., eds. (1994) Current Protocols in Molecular Biology, vol. 3, p. A.1C.3 Haq T A, Mason H S, Clements J D, Arntzen C J (1995) Oral immunization with a recombinant bacterial antigen produced in transgenic plants. Science 268:714-716). The designed sequence for HN is shown in FIG. 1. The synthetic HN gene was assembled by a commercial supplier (Retrogen) and was received in two separate plasmids containing either the 5′ (p4187-4203-1) or 3′ (p42111-4235-1c-1) half of the gene cloned into pCR-Blunt.

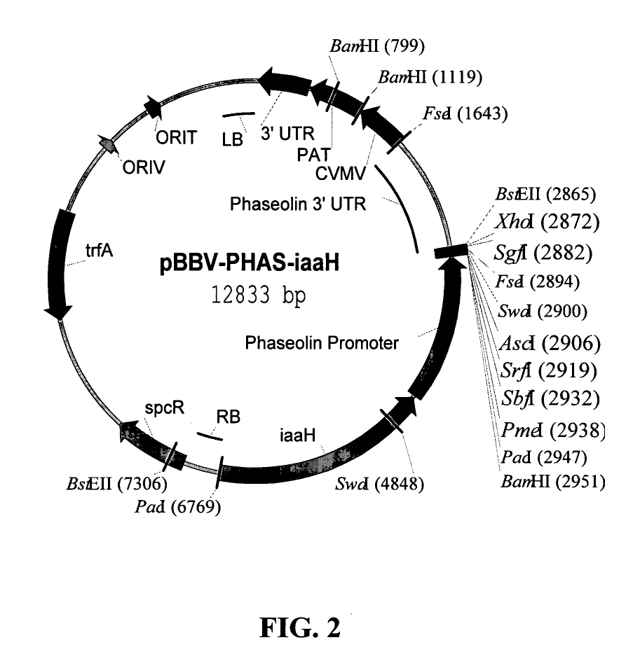

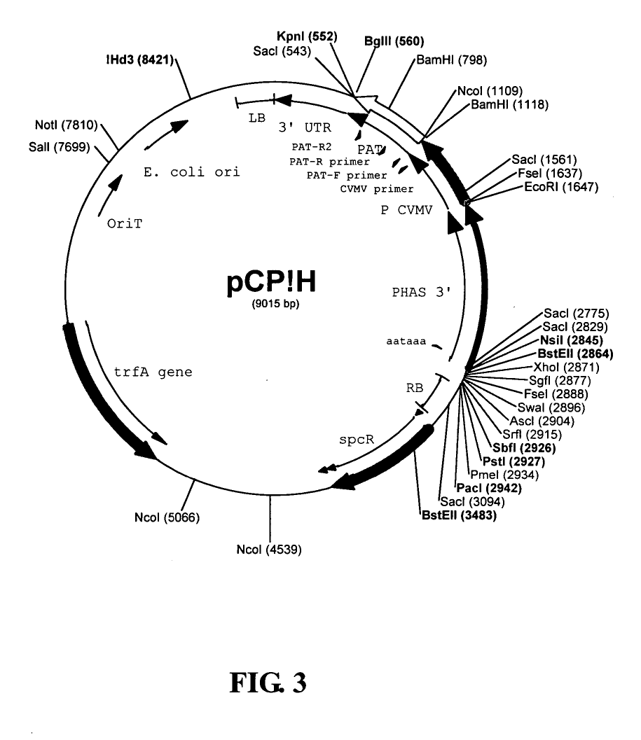

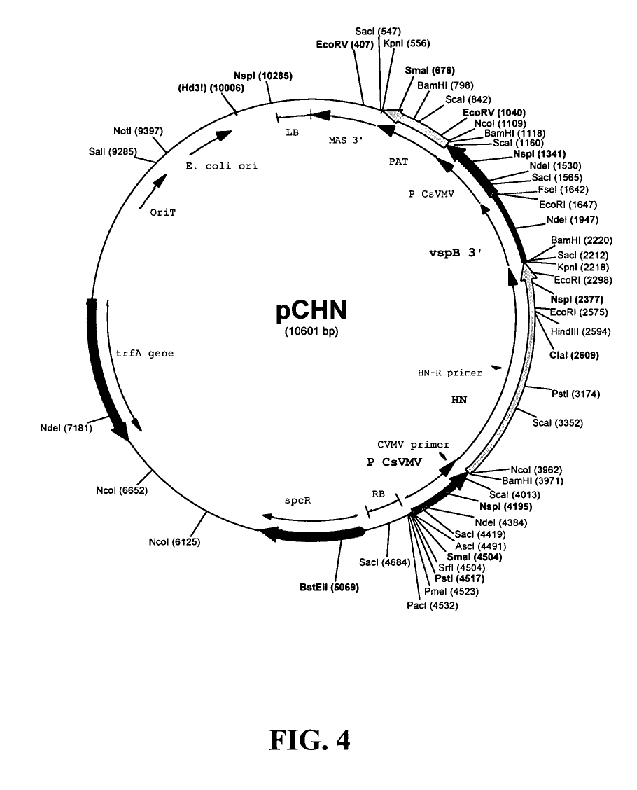

Plasmid construction: Binary vectors for Agrobacterium-mediated plant transformations were constructed based on vector pBBV-PHAS-iaaH shown in FIG. 2, which uses the plant selection marker phosphinothricin acetyl transferase (PAT), described in U.S. Pat. Nos. 5,879,903; 5,637,489; 5,276,268; and 5,273,894 herein incorporated by reference, driven by the constitutive cassaya vein mosaic virus promoter (CsVMV) described in WO 97/48819. We first deleted the iaaH gene and the phaseolin promoter sequence by digestion of pBBV-PHAS-iaaH with PacI and re-ligating to form pCVMV-PAT; then we deleted the single HindIII site by filling it with Klenow enzyme and re-ligating to form pCP!H. We end-tailored the CsVMV promoter by PCR using primers CVM-Asc (5′-ATGGCGCGCCAGAAGGTAATTATCCAAG SEQ ID NO:5) and CVM-Xho (5′-ATCTCGAGCCATGGTTTGGATCCA SEQ ID NO:6) on template pCP!H, and cloned the product in EcoRV-digested, T-tailed pBluescriptKS to make pKS-CVM7. A map of pCP!H is shown in FIG. 3. We constructed the HN expression cassette pKS-CHN by ligating the vector pKS-CVM7/NcoI-EcoRI with 3 insert fragments: the HN 5′ half on NcoI/PstI, the HN 3′ half on PstI/KpnI, and the soybean vspB 3′ element on KpnI-EcoRI (Haq 1995). The binary T-DNA vector pCHN was then assembled by ligation of the vector pCP!H/AscI-EcoRI and the AscI-EcoRI fragment of pKS-CHN. A map of pCHN is shown in FIG. 4.

The granule bound starch synthase (GBSS) promoter, described in U.S. Pat. No. 5,824,798 herein incorporated by reference, was used to make other vectors. These constructs were made using a promoter fragment amplified from genomic DNA of Solanum tuberosum L. cv. “Desiree” using primers designed from the sequence in Genbank accession X83220 for the Chinese potato cultivar “Dongnong”. A mutagenic primer “GSS-Nco” (5′-[TGCCATGGTGATGTGTGGTCTACAA] SEQ ID NO:7) was used to create a Nco I site overlapping the translation initiation codon, along with forward primer “GSS-1.8F” (5′-[GATCTGACAAGTCAAGAAAATTG] SEQ ID NO:8) complimentary to the 5′ region at −1800 bp; the 1825 bp PCR product was cloned in T-tailed pBluescriptKS to make pKS-GBN, and sequenced. A mutagenic primer “GSS-Xho” (5′-[AGCTCGAGCTGTGTGAGTGAGTG] SEQ ID NO:9) was used to create a XhoI site just 3′ of the transcription start site along with primer “GSS-1.8F”; the 1550 bp PCR product was cloned in T-tailed pBluescriptKS to make pKS-GBX, and sequenced.

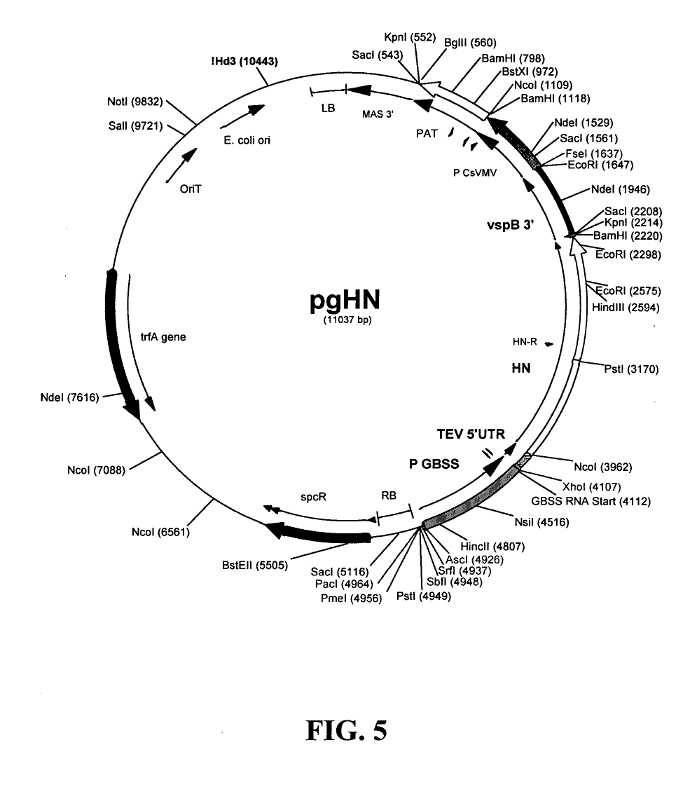

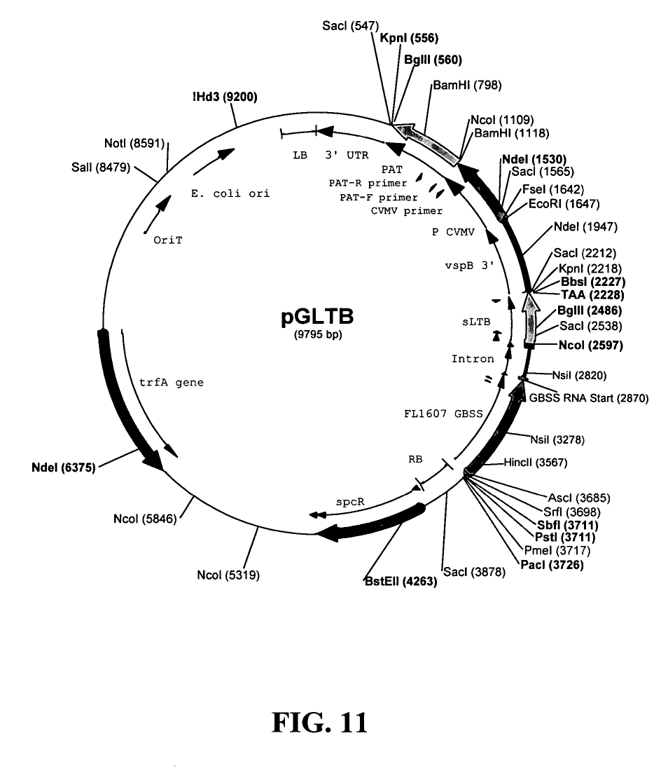

A GBSS promoter expression cassette containing the TEV 5′UTR (untranslated region), described in U.S. Pat. No. 5,891,665 herein incorporated by reference, was assembled by ligation of vector pTH210 digested with HindIII/XhoI with the HindIII/XhoI fragment of pKS-GBX, which effected a substitution of the CaMV 35S promoter with the 811 bp GBSS promoter, to make pTH252A. See Haq T A, Mason H S, Clements J D, Arntzen C J (1995) Oral immunization with a recombinant bacterial antigen produced in transgenic plants. Science 268:714-716. The HN gene was inserted into pTH252A/NcoI-KpnI by ligation with the HN 5′ half on NcoI/PstI and the HN 3′ half on PstI/KpnI to make pHN252A. The binary T-DNA vector pgHN was made by ligation of the vector pGLTB (shown in FIG. 11) digested with NsiI and EcoRI with the fragments pHN252A/NsiI-KpnI and pTH210/KpnI-EcoRI. A map of pgHN is shown in FIG. 5.

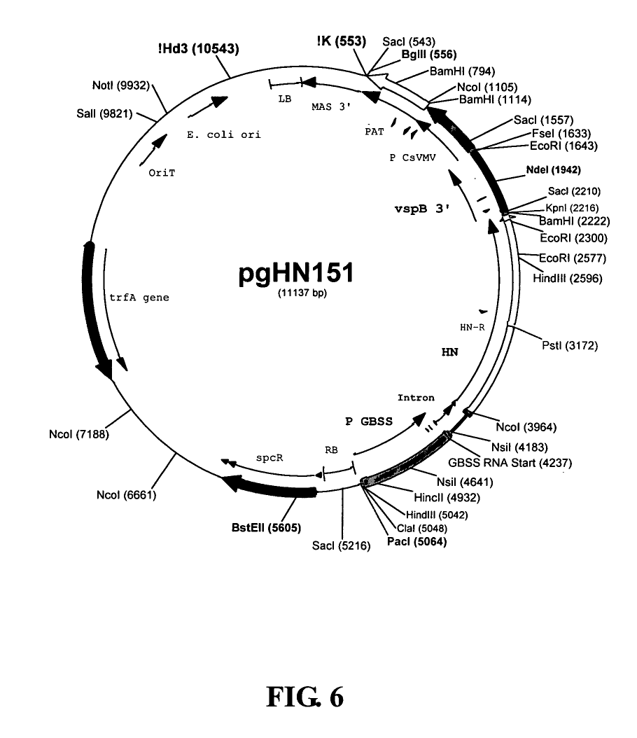

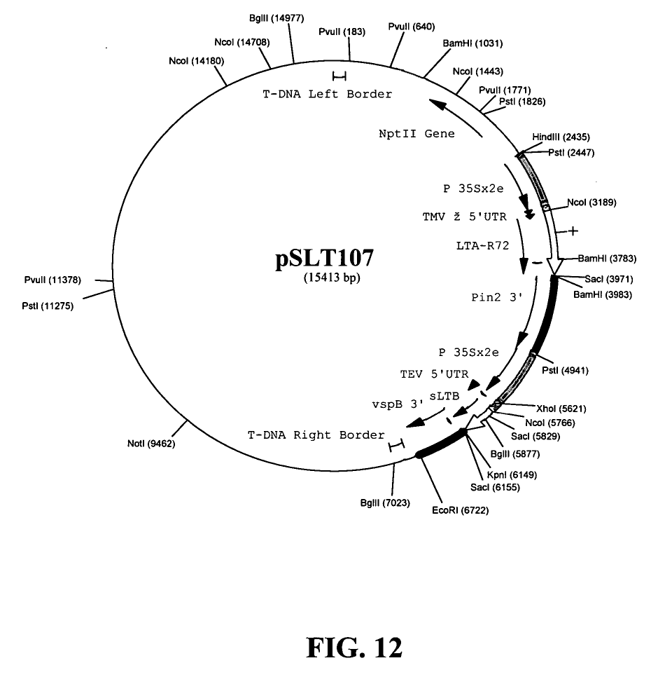

A GBSS promoter expression cassette containing the GBSS 5′UTR, described in U.S. Pat. No. 5,824,798, herein incorporated by reference, with its intron was assembled by ligation of vector pTH210 (Haq 1995) digested with HindIII/NcoI with the HindIII/NcoI fragment of pKS-GBN, which effected a substitution of the (cauliflower mosaic virus) CaMV 35S promoter/TEV 5′UTR with the 1084 bp GBSS promoter/5′-UTR, to make pTH251A. The binary T-DNA vector pgHN151 was made by ligation of the vector pCLT105 (shown in FIG. 12) with fragments pTH251A/HindIII-NcoI and pHN252A/NcoI-KpnI. A map of pgHN151 is shown in FIG. 6.

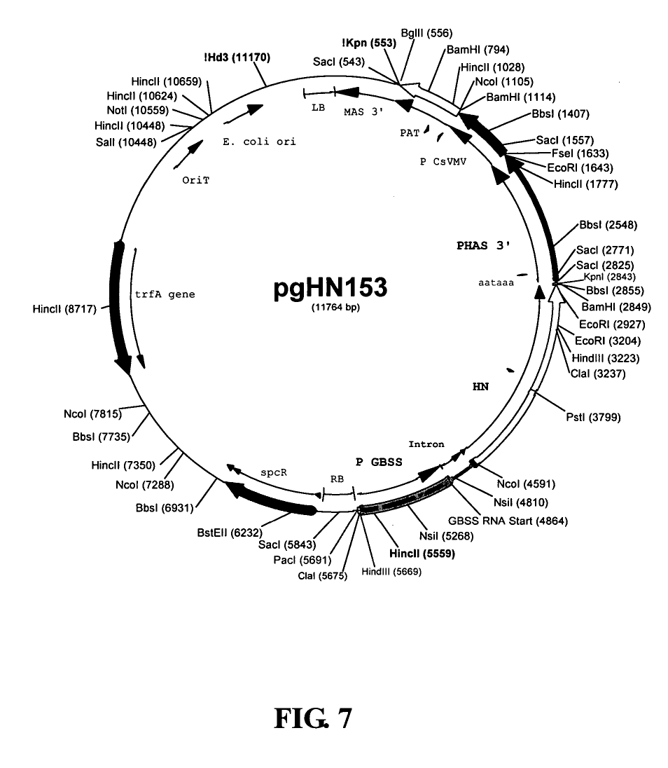

A GBSS promoter expression cassette containing the GBSS 5′UTR with its intron and the bean phaseolin 3′ element (described in U.S. Pat. Nos. 5,270,200; 6,184,437; 6,320,101, herein incorporated by reference) was constructed. First, pCP!H was digested at the unique KpnI site, blunted with T4 DNA polymerase, and re-ligated to make pCP!HK, which has the KpnI site removed. pCP!HK was digested with NsiI, followed by blunting with T4 DNA polymerase, and then digestion with PacI. The resulting vector was ligated with a 2848 bp fragment from pgHN151 digested with SacI, followed by blunting with T4 DNA polymerase, and then digestion with PacI, to make pgHN153. A map of pgHN153 is shown in FIG. 7.

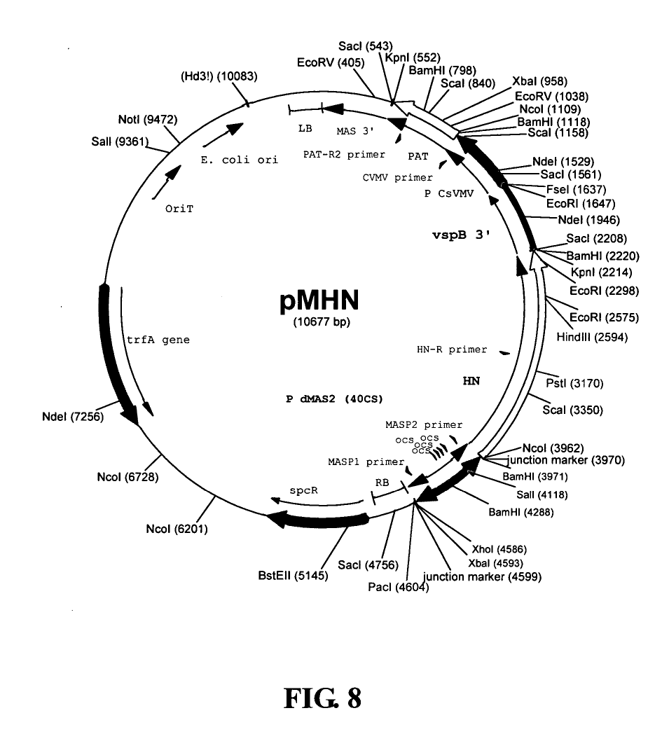

A chimeric constitutive promoter (4OCSΔMAS U.S. Pat. Nos. 5,001,060; 5,573,932 and 5,290,924 herein incorporated by reference) was used to construct another expression vector for HN. Plasmid, pAGM149, was digested with EcoRV and partial digestion with BamHI. This fragment was ligated with pCHN digested with PmeI/PstI and the 5′ half of the synthetic HN gene obtained by digestion of pKS-CHN with BamHI/PstI. The resulting pMHN is shown in FIG. 8.

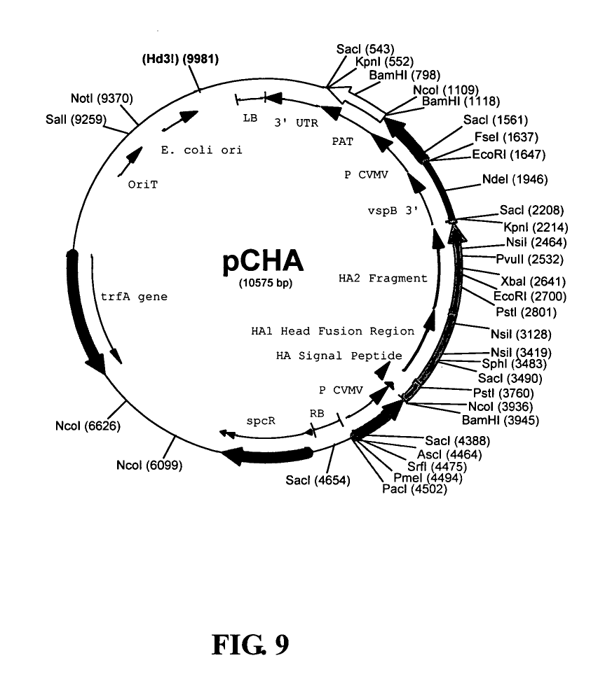

A plasmid containing the HA gene of AIV A/turkey/Wisconsin/68 (H₅N₉) was obtained from David Suarez (SEPRL, Athens, Ga.) (FIG. 10). It was end-tailored by PCR to add restriction sites NcoI at 5′ and KpnI at 3′ end, and inserted into the vector pIBT210.1 (Haq et al., 1995), containing the 35S promoter, TEV 5′-UTR, and vspB 3′ end. The expression cassette was transferred to the binary vector pGPTV-Kan (Becker et al., Plant Mol Biol 1992; 20: 1195-7) by digestion with HindIII and EcoRI (partial), to make pIBT-HAO. The HA gene/vspB3′ end fragment from pIBT-HAO was obtained by digestion with NcoI and EcoRI (partial), and inserted into pKS-CVM7 to make pKS-CHA. The cassette containing the CsVMV promoter, HA gene, and vspB3′ end was obtained from pKS-CHA by digestion with AscI and EcoRI (partial), and ligated with pCP!H to make pCHA, shown in FIG. 9.

The plant-optimized sequence encoding the LT-B gene of E. coli strain H10407 is know in the art (Mason H S, Haq T A, Clements J D, Arntzen C J, 1998, Vaccine 16:1336-1343). The plant-optimized sequence encoding the LT-A gene of E. coli strain HI 0407 was described in WO/00/37609 which was originally filed as U.S. Provisional Application No. 60/113,507, the entire teachings of which are herein incorporated by reference. WO/00/37609 describes the construction of three binary T-DNA vectors (pSLT102, pSLT105, pSLT107) that were used for Agrobacterium tumefaciens-mediated plant cell transformation of Nicotiana tabacum NT-1 cells in Example 2. The resulting transformed NT-1 cell lines (SLT102, SLT105 and SLT107) expressed and accumulated fully assembled LT and LT analogs comprised of LT-B and modified forms of the LT-A subunit. FIG. 12 illustrates pSLT107, which contains a modified LT-A gene that replaces Ala72 with Arg72. SLT102 and SLT105 expression products were identical except that they contained different alterations in the LT-A gene (Ser63 to Lys63 in pSLT102; Arg192 to Gly192 in pSLT105. These lines contain an undetermined number of copies of the T-DNA region of the plasmids stably integrated into the nuclear chromosomal DNA. The transgenic NT1 cells accumulated LT-B subunits that assembled into ganglioside-binding pentamers, at levels up to 0.4% of total soluble protein as determined by ganglioside-dependent ELISA. The transgenic NT1 cells also accumulated modified LT-A subunits, some of which assembled with LT-B pentamers as determined by ganglioside-dependent ELISA using LT-A specific antibodies.

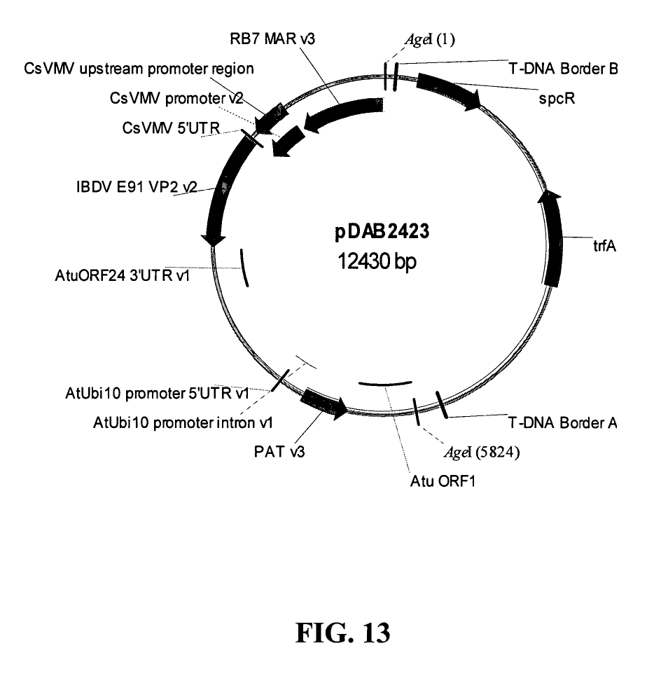

A binary vector for Agrobacterium-mediated plant cell transformation was constructed from basic binary vector (PBBV) modified at the unique BamHI site with an AgeI linker for addition of a VP2 and selectable marker expression cassette. VP2 is flanked by an RB7 MAR element (U.S. Pat. No. 5,773,689; U.S. Pat. No. 5,773,695; U.S. Pat. No. 6,239,328, WO 94/07902, and WO 97/27207) and the CsVMV promoter, with Agrobacterium tumifaciens (Atu) ORF 24 (GenBank accession number X00493) 3′UTR. The selectable marker, PAT, is regulated by Arabidopsis thaliana (At) Ubiquitin 10 promoter (Plant J. 1997. 11(5):1017; Plant Mol. Biol. 1993. 21(5):895; Genetics.1995. 139(2):921) and Atu ORF 1 (US5428147; Plant Molecular Biology. 1983. 2:335; GenBank accession number X00493) 3′ UTR; the resulting plasmid pDAB2423 is shown in FIG. 13.

Infectious Bursal Disease (IBD) virus, very virulent strain Ehime 91 (J Vet Med Sci. 1992. 54(1):153; JVI. 2002. 76(11):5637) was used to produce the VP2 plant-optimized nucleotide sequence, based on reported VP2 amino acid sequence (GenBank accession number AB024076), with amino acids #454-456 from strain UK661 (GenBank accession number NC_{—}004178). (See FIG. 14 for VP2 sequence).

### EXAMPLE 2

### Preparation of Transgenic Nicotiana tabacum

Three to 4 days prior to transformation, a 1 week old NT-1 culture was sub-cultured to fresh medium by adding 2 ml of the NT-1 culture into 40 ml NT-1 media. The sub-cultured was maintained in the dark at 25±1° C. on a shaker at 100 rpm.

NT-1 Medium

| Reagent | Per liter | |
|---|---|---|
| MS salts | 4.3 | g |
| MES stock (20×) | 50 | ml |
| B1 inositol stock (100×) | 10 | ml |
| Miller's I stock | 3 | ml |
| 2,4-D (1 mg/ml) | 2.21 | ml |
| Sucrose | 30 | g |
| pH to 5.7 ± 0.03 | | |

B1 Inositol Stock (100×)(1 liter)

Thiamine HCl (Vit B1)-0.1 g

MES (20×) (1 liter)

MES (2-N-morpholinoethanesulfonic acid)—10 g

Myoinositol—10 g

Miller's I (1 liter)

KH₂PO₄—60 g

| | Per 1 liter | |
|---|---|---|
| MS Basal Salts | DI water | |
| Modified MS vitamins (100×) | 10 | ml |
| Myo-inositol | 100 | mg |
| Potassium Phosphate | 137.4 | g |
| Dibasic Anhydrous | | |
| MES | 0.5 | g |
| 2,4-D (10 mg/ml) | 222 | ul |
| Sucrose | 30 | g |
| L-Proline | | |

| | Per Liter | |
|---|---|---|
| Modified MS vitamins | DI water | |
| Nicotinic Acid | 5 | mg/L |
| Pyridoxin HCL | 50 | mg/L |
| Thiamine HCL | 200 | mg/L |
| Glycine | 200 | mg/L |

2.5 M L-Proline Stock

M.W = 115.1 grams/L
Prepare 100 ml of 2.5 M Stock
115.1/10 = 11.51 × 2.5 = 28.775 grams in 100 mls

Agrobacterium tumefaciens containing the expression vector of interest was streaked from a glycerol stock onto a plate of LB medium containing 50 mg/l spectinomycin. The bacterial culture was incubated in the dark at 30° C. for 24 to 48 hours. One well-formed colony was selected, and transferred to 3 ml of YM medium containing 50 mg/L spectinomycin. The liquid culture was incubated in the dark at 30° C. in an incubator shaker at 250 rpm until the OD₆₀₀ was 0.5-0.6. This took approximately 24 hrs.

LB Medium

| Reagent | Per liter |
|---|---|
| Bacto-tryptone | 10 g |
| Yeast extract | 5 g |
| NaCl | 10 g |
| Difco Bacto Agar | 15 g |

YM Medium

| Reagent | Per liter |
|---|---|
| Yeast extract | 400 mg |
| Mannitol | 10 g |
| NaCl | 100 mg |
| MgSO₄.7H₂0 | 200 mg |
| KH₂PO₄ | 500 mg |

(Alternatively, YM in powder form can be purchased (Gibco BRL; catalog #10090-011). To make liquid culture medium, add 11.1 g to 1 liter water.)

On the day of transformation, 1 μl of 20 mM acetosyringone was added per ml of NT-1 culture. The acetosyringone stock was made in ethanol the day of the transformation. The NT-1 cells were wounded to increase the transformation efficiency. For wounding, the suspension culture was drawn up and down repeatedly (20 times) through a 5 ml wide-bore sterile pipet. Four milliliters of the suspension was transferred into each of 10, 60×15 mm Petri plates. One plate was set aside to be used as a non-transformed control. Approximately, 50 to 100 μl of Agrobacterium suspension was added to each of the remaining 9 plates. The plates were wrapped with parafilm then incubated in the dark on a shaker at 100 rpm at 25±1° C. for 3 days.

Cells were transferred to a sterile, 50 ml conical centrifuge tube, and brought up to a final volume of 45 ml with NTC medium (NT-1 medium containing 500 mg/L carbenicillin, added after autoclaving). They were mixed, then centrifuged at 1000 rpm for 10 min in a centrifuge equipped with a swinging bucket rotor. The supernatant was removed, and the resultant pellet was resuspended in 45 ml of NTC. The wash was repeated. The suspension was centrifuged, the supernatant was discarded, and the pellet was resuspended in 40 ml NTC. Aliquots of 5 ml were plated onto each Petri plate (150×15 mm) containing NTCB10 medium (NTC medium solidified with 8 g/l Agar/Agar; supplemented with 10 mg/l bialaphos, added after autoclaving). Plates were wrapped with parafilm then maintained in the dark at 25±1 C. Before transferring to the culture room, plates were left open in the laminar flow hood to allow excess liquid to evaporate. After 6 to 8 weeks, putative transformants appeared. They were selected and transferred to fresh NTCB5 (NTC medium solidified with 8 g/l Agar/Agar; supplemented with 5 mg/l bialaphos, added after autoclaving). The plates were wrapped with parafilm and cultured in the dark at 25+1° C.

Putative transformants appeared as small clusters of callus on a background of dead, non-transformed cells. These calli were transferred to NTCB5 medium and allowed to grow for several weeks. Portions of each putative transformant were selected for ELISA analysis. After at least 2 runs through ELISA, lines with the highest antigen levels were selected. The amount of callus material for each of the elite lines was then multiplied in plate cultures and occasionally in liquid cultures.

### EXAMPLE 3

### Antigen Preparation

Cells are removed from the fermentor via the harvest port using a peristaltic pump and silicone tubing. The cells are pumped over a conical filter apparatus containing 30 um Spectramesh and the cells are filtered to a wet cell cake via vacuum. The cells are then suspended in cold lysis buffer containing Dulbecco's Phosphate Buffered Saline (catalogue # 21-031-CV Mediatech, Inc) with 1 mM ethlenediaminetetraacetic acid (EDTA; catalogue number is E(884, Sigma Aldrich) at a ratio of 2 ml of buffer per gram of filtered cells. The cell slurry is held at 5° C. until processed. Prior to microfluidization the cells can be homogenized using a Silverson L4RT Mixer at 6000 rpm for 5-10 minutes. The Microfluidics model 110L microfluidizer fitted with a 100 um Z configuration interaction chamber (H10Z) is primed with approximately 200 ml of cold lysis buffer. The chamber pressure is set to 18,000 PSI and the interaction chamber: inlet and output lines are covered with ice. The sample is passed through the microfluidizer at a flow rate of 100 ml/min and the lysed cell suspension collected on ice. The processed solution is clarified of cellular debris by centrifugation at 2800×g for 15 minutes at 4° C. Supernatant, with released HN, HA, LT or VP2 protein, is separated from the cellular debris pellet and stored at −80° C. The 2800×g pellet is resuspended in a 2-fold excess of fresh lysis buffer and incubated for 16 hours at 5° C. to extract HN proteins that remain associated with the cellular debris. The cellular debris is pelleted at 2800×g for 15 minutes at 4° C. in a swinging bucket rotor. The supernatant is decanted and stored at −80° C.

Processing from cold storage is performed by centrifuging the bulk material at 2800×g for 15 minutes at 4° C., the supernatant is filtered through a 30 um Spectramesh under vacuum. The supernatant bulk material can be concentrated using a Pall Centramate tangential flow system using a molecular weight cutoff membrane smaller than the product target molecular weight. The inlet and retentate lines are directed to the product vessel and the product pool is concentrated 10-20 fold. The permeate is tested for breakthrough of the product. Upon completion of the concentration, the Centramate unit is flushed with 500 ml of DPBS and this wash is added to the final concentrate pool. The concentrate is stored at 4° C. or −80° C.

For sonication whole wet NT-1 cells expressing either HN, HA or null control were harvested directly from cell culture and filtered to remove excess media by placing a Spectramesh 30 filter in a Buchner funnel and pouring cells and media through the filter using a slight vacuum. 0.5 grams of cells were placed in 2 mls of buffer (Dulbecco's Phosphate Buffered Saline and 1 mM EDTA), and then sonicated for 15 to 20 seconds on ice. Sonication was performed using a Branson 450 sonifier with a replaceable microtip at output control of 8, duty cycle 60 for varying amounts of time. Sonicates were then placed on ice until use. For larger masses of cells the time needed for sonication will increase proportionately (for example, for 250 g of cells, sonication will be increased to 8-10 minutes).

### EXAMPLE 4

### Antigen Extraction

To examine whether non-detergent treatments could release ELISA signal from transformed NT-1 cells and allow retention of biological activity, a series of treatments were set up that involved comparison of treatments without detergent and various levels of sonication or microfluidization. The results were striking in that periods of sonication greater than 20 seconds in extraction buffer completely destroys hemagglutination activity of HN from a pCHN bearing NT-1 cell line, but not ELISA signal. In contrast, sonication for only 20 seconds in DPBS not only released antigen detectable by ELISA signal, but the soluble protein extracts demonstrated excellent hemagglutination activity (see Table 1).

Plant-derived HN extracted without harsh detergents or detergents at high concentration was used as the antigen in hemagglutination inhibition assays to determine if polyclonal antibody produced to native virus could recognize and inhibit agglutination of RBC's by the plant-derived HN. The results indicate that native antibody will recognize the hemagglutination epitope of the plant-derived HN in a similar manner as native virus (Table 2). The data from Table 2 also demonstrates that control NT-1 cells or NT-1 cells expressing a non-hemagglutinating protein do not agglutinate red blood cells nor are affected by NDV specific serum. In this experiment, extracts of plant-derived protein were diluted to 4 HA (hemagglutination) units, and then treated with NDV specific polyclonal antisera. Four HA units are the standard amount of virus used for titration of serum.

The above data demonstrates that using an extraction method that does not utilize harsh detergent and reduces the amount of cell disruption produces an extracellular fraction that retains hemagglutination activity for transformed NT-1 cell lines expressing HN or HA. To determine if HN protein from non-detergent extracted NT-1 cells had additional biological activity that may be relevant to vaccine efficacy, the HN extracts were examined for ability to bind to chicken cell receptors. Immunofluorescence staining indicated that chicken embryo fibroblast (CEF) cells treated with native virus or pCHN-18 extracts were indistinguishable. Thus, plant-derived HN retains virus-like ability to bind to receptors on target cell surfaces.

The combined data from Tables 1 and 2 together with the hemagglutination and immunofluorescence assays discussed above suggest that the HN protein derived from transgenic NT-1 cells of the present invention retains both immunological and biological features. Also, proteins and immunoprotective particles can be released from the plant cell in an efficacious and native form in the absence of detergents. Most significant of the data provided above is that antisera to native virus will recognize plant-derived HN in HAI tests. Chickens that contain at least 4-fold higher titer of hemagglutination inhibition (HAI) activity above background are almost always certain of protection against challenge from virulent virus.

To examine whether the yield of HN could be increased by other means of mechanical disruption the cells were exposed to microfluidization as described above. Various pressures were used to examine the effect of disruption and biological activity of the HN protein; Table 3 shows the results of the study. The data suggest that the amount of hemagglutination activity per unit mass of HN protein can change more than 10 fold using this method of disruption, however, the protein concentration only increases about 20%. These data suggest that the HN protein is integrated into larger particle sizes that are only partially released from sonication and that smaller particle sizes can exist that retain biological activity. Using a disruption method that produces a more homogenous extract can result in the recovery of additional active polypeptide.

### EXAMPLE 5

### Quantitative ELISAs

Quantitative ELISA VP2

Nunc Maxisorp 96-well microtiter ELISA plates were coated with Chicken anti-IBDV polyclonal antiserum (SPAFAS Lot No. G0148) diluted 1:2000 in 0.01 M borate buffer using 100 μl per well; plates were incubated at 5° C. overnight. The plates were washed 3 times with 300 μl/well PBS-T (1×PBS containing 0.05% Tween 20, Sigma Cat. No. P-1379). Each well was then incubated one hour at 37° C. with 200 μl of blocking buffer (5% (w/v) non-fat dried milk, Difco Cat. No. 232100 in PBS). The wells were washed 3× with 300 μl/well using PBS-T. IBDV reference antigen (BEI inactivated IBDV D-78 strain Lot No. 2209031BDV) was diluted to a final concentration of 1000 ng/ml VP2 in blocking buffer. Samples were pre-diluted in blocking buffer. The diluted reference antigen and experimental antigen samples were added to the plate by applying 200 μl of sample to duplicate wells in row B and 100 μl of blocking buffer to remaining wells. Serial 2 fold dilutions were made by mixing and transferring 100 μl per well, 6 dilutions per reference or sample. Plates were then incubated 1 hour at 37° C., washed 3× in PBS-T and 100 μl of R-63 monoclonal antibody ascites fluid (IBDV VP2 specific Lot No. 190903R-63) diluted 1:10,000 in blocking buffer was added per well and incubated 1 hour at 37° C. The plates were washed 3× with PBS-T. Goat anti-Mouse IgG peroxidase-labeled antibody conjugate (KPL Cat. No. 074-1806) diluted 1:2000 in blocker was added at 100 μl/well and plates were incubated 1 hour at 37° C. The plates were washed 3× in PBS-T and 100 μl of ABTS substrate (KPL Cat. No. 50-66-01) was added to each plate and incubated at room temperature for approximately 5 minutes. Optical density at 405 nm wavelength was determined using a Tecan Sunrise Plate reader. Data were transported and displayed using Tecan Magellan Software. Linear regression and quantitation analysis were done using Microsoft Office Excel 2003.

Nunc Maxisorp 96-well microtiter ELISA plates were coated with 5 μg/well of mixed GM1 ganglioside in 0.01 M borate buffer using 100 μl per well; plates were incubated at room temperature overnight. The plates were washed 3 times with PBS-T (1× containing 0.05% Tween 20, Sigma, Lot No.120K0248). Each well was then incubated one hour at 37° C. with 200 μl of blocking buffer containing 5% (w/v) non-fat dried milk in PBS-0.05% Tween 20. The wells were washed 3× with 250 μl/well using PBS-T. LT reference antigen [or LT-B reference antigen were diluted to 50 ng/ml. Samples were pre-diluted in blocking buffer. The diluted reference antigen and samples were added to the plate by applying 200 μl of sample in row A and 100 μl of blocking buffer to remaining rows. Serial 2-fold dilutions were made by mixing and transferring 100 μl per well. Plates were then incubated 1 h at 37° C., washed 3× in PBS-T and 100 μl of diluted LT-A or LT-B specific antisera in blocking buffer was added per well and incubated 1 h at 37° C. The plates were washed 3× in PBS-T and then 100 μl of peroxidase-labeled antibody conjugate was added and incubated for 1 hour at 37° C. The plates were washed 3× in PBS-T and 50 μl of TMB substrate was added to each plate. TMB stop solution was added at 20 minutes post addition of substrate. Optical density at 450 nm wavelength was determined using a Tecan Sunrise Plate reader. Data were transported and displayed using Tecan Magellan Software. Linear regression and quantitation analysis were done using Microsoft Excel 2000 version 9.0.3821 SR-1.

Quantitative ELISA HN

Quantitative ELISA for HN can be performed by coating the plates on the day prior to running the assay. 50 μl per well of Capture Antibody (Rabbit anti-HN in 50% glycerol, diluted (1:500) in 0.01M Borate Buffer) is added to each well of each flat bottom 96-well microtiter plate. Cover the plate and incubate at 2° C.-7° C. overnight, (12-18 hours). The coated ELISA plate(s) should be allowed to equilibrate to room temperature (approximately 20-30 minutes) and then washed three times with 200-300 μl per well per wash with PBS-T. Block the entire plate to prevent non-specific reactions by adding 200 μl per well of 3% Skim Milk Blocking Solution. The plate(s) is(are) then incubated for 2 hours (+10 minutes) at 37° C.±2° C. (covered with a plate cover or equivalent). Add HN Reference antigen (Ag) in 1% Skim Milk Blocker to a concentration of 250 ng HN/ml; experimental antigens are diluted in 1% Blocker. Wash the HN ELISA plate(s) one time with PBS-T and add 100 μl per well of diluted HN Reference Antigen and HN Test Samples to Row B; add 50 μl per well of 1% Blocker to all remaining wells; serially dilute the samples down the plate by transferring 50 μl per well from row B to row G, mixing 4-5 times with the pipette before each transfer. Cover plate(s) and incubate 1 hour (+10 minutes) at 37° C.±2° C.; wash the ELISA plate(s) three times with PBS-T. Fifty NDV HN 4A Ascites Fluid in 50% glycerol (1:2000) in 3% Blocker is added to each well and the plates are covered and incubated 1 hour (+10 minutes) at 37° C.±2° C. The ELISA plate(s) are washed three times with PBS-T and 50 μl of rabbit anti-Mouse IgG in 50% glycerol (1:3000) in 3% Blocker is added to each well; the plates are covered and incubated 1 hour (+10 minutes) at 37° C.±2° C. ELISA plate(s) are washed three times with PBS-T and 50 μl of ABTS Peroxidase Substrate Solution (equilibrated at RT (room temperature) for at least 30 minutes) is added to each well. Cover plate(s) and incubate at RT in the dark for 15-20 minutes. The Optical Density (OD) of the wells is read at a wavelength of 405 nm (with a 492 nm Reference Filter). The initial dilution of the HN Reference Antigen should be within 0.7-1.0 OD, this serves as the positive control for the ELISA.

Quantitative ELISA HA

For quantitative ELISA of HA, coat the plates on the day prior to running the assay. Fifty μl per well of Capture Antibody (goat anti-Hav5 in 50% glycerol, diluted (1:1000) in 0.01M Borate Buffer) is added to each well of flat bottom 96-well microtiter plate(s)). Cover the plate(s) and incubate at 2° C.-7° C. overnight, (12-18 hours). The coated ELISA plate(s) is(are) allowed to equilibrate to room temperature (approximately 20-30 minutes) and is(are) then washed three times with 200-300 μl per well per wash with PBS-T. The entire plate is blocked to prevent non-specific reactions by adding 200 μl per well of 3% Skim Milk Blocking Solution. The plate(s) is(are) then incubated for 2 hours (+10 minutes) at 37° C.±2° C. (covered with a plate cover or equivalent). AIV-HA (allanotoic fluid) reference Antigen is added in 1% Skim Milk Blocker to a concentration of 1000 ng HA/ml; experimental antigens are diluted in 1% Blocker. The HA ELISA plate(s) are washed one time with PBS-T and 100 μl per well of diluted HA reference antigen and HA Test Samples are added to Row B; add 50 μl per well of 1% Blocker to all remaining wells; serially dilute the samples down the plate by transferring 50 μl per well from row B to row G, mixing 4-5 times with the pipette before each transfer. Cover plate(s) and incubate 1 hour (+10 minutes) at 37° C.±2° C.; wash the ELISA plate(s) three times with PBS-T. Fifty μl of chicken anti-AIV polyclonal antisera in 50% glycerol (1:2000) in 3% Blocker is added to each well and the plates are covered and incubated 1 hour (+10 minutes) at 37° C.±2° C. Wash the ELISA plate(s) three times with PBS-T and then add 50 μl of goat anti-chicken IgG in 50% glycerol (1:3000) in 3% Blocker to each well; the plates are covered and incubated 1 hour (+10 minutes) at 37° C.±2° C. Wash the ELISA plate(s) three times with PBS-T and add 50 μl of ABTS Peroxidase Substrate Solution (equilibrated at RT for at least 30 minutes) to each well. Cover plate(s) and incubate at RT in the dark for 15-20 minutes. The Optical Density (OD) of the wells read at a wavelength of 405 nm (with a 492 nm Reference Filter). The initial dilution of the HA Reference Antigen should be within 0.7-1.0 OD, this serves as the positive control for the ELISA.

Quantitative ELISA LT and LTB

Nunc Maxis 96-well microtiter ELISA plates were coated with 5 ug/well of mixed GM1 ganglioside in 0.01 M borate buffer using 100 μl per well; plates were incubated at room temperature overnight. The plates were washed 3 times with PBS-T. Each well was then incubated one hour at 37° C. with 200 μl of blocking buffer containing 5% (w/v) non-fat dried milk in PBS-T. The wells were washed 3× with 250 μl/well using PBS-T. Reference antigen and sample antigens were mixed 1:1 with PBS-T before adding to plates. LT reference antigen and LTB reference antigen were diluted to 50 ng/ml in the first well. Samples were added to the plate by applying 200 μl of sample in row A and 100 μl of blocking buffer to remainder rows. Serial 2 fold dilutions were made by mixing and transferring 100 μl per well. Plates were then incubated 1 h at 37° C., washed 3× in PBS-T and 100 μl of diluted antisera in blocking buffer was added per well and incubated 1 h at 37° C. The plates were washed 3× in PBS-T and then 100 μl of antibody conjugate was added and incubated 1 h at 37° C. The plates were washed 3× in PBS-T and 50 μl of TMB substrate was added to each plate and TMB stop solution was added at 20 minutes post addition of substrate. Optical density at 450 nm wavelength was determined using a Tecan Sunrise Plate reader. Data were transported and displayed using Tecan Magellan Software, Linear regression and quantitation analysis were done using Microsoft Excel 2000 version 9.0.3821 SR-1.

### EXAMPLE 6

### Serum ELISAs

Serum ELISA LT

Blood was collected by decapitation (birds 0-7 days of age) or by venipuncture in the wing web or jugular vein. Birds were euthanized by cervical dislocation or by CO₂ exposure for 1-5 minutes prior to decapitation. The blood was transported from the animal facility to the laboratory and placed at 2-7° C. for 45 minutes to advance and condense the blood clot. The blood samples were transferred to a 37° C. water bath for 10 minutes and then centrifuged for 20 minutes at 2500 rpm using a Beckman GPR centrifuge at 2-7° C. The serum was aseptically removed from each tube, 0.5-1.5 ml was aliquoted to a cryotube (Nunc) and stored at −18° C. until used. For serum ELISA, the ganglioside adsorption step utilized 1.5 μg/well or 15 μg/ml with incubation overnight at 2-7° C. The plates were washed 3× with PBS-T and then blocked for 1 hour at 37° C. with 3% skim milk PBS. To titer antibody per serum sample, after the ganglioside is adsorbed, 100 μl of LT-B or LT at 2.5 ug/ml in blocking buffer is added per well and incubated 1 hour at 37° C. The plates were washed 3× with PBS-T and then 200 μl of the serum sample diluted in blocking buffer was added to Row A and 100 μl of blocking buffer was added to the remaining rows. Starting dilution for serum was 1:10 in blocking buffer unless specified otherwise. After two-fold serially dilutions of the serum samples, the plates were incubated 1 hour at 37° C. and then washed 3× in PBS-T. The goat anti-chicken conjugate was labeled with HRP were added and incubated 1 hour at 37° C. Plates were washed and 100 μl of ABTS was added and incubated until the positive control provided a 0.7 to 1.0 absorbance at 405/492 dual wavelength using a Tecan Sunrise Plate reader. Data was transported and displayed using Tecan Magellan Software. Linear regression and quantitation analyses were done using Microsoft Excel 2000 version 9.0.3821 SR-1. The serum geometric mean titer (GMT) was determined for each treatment group using Microsoft Excel 2000 version 9.0.3821 SR-1. Background ELISA titers of <10 were given a value of 1 for these calculations. Difference in least squares means for treated birds from controls was determined using least squares analysis. A treatment was passed as effective if there was a significant difference of a treatment group with the non-vaccinated non-challenged control group.

Serum ELISA NDV-HN

Coat plates with rabbit A-NDV pooled antiserum (Mixed 1:2 with 50% glycerol in water) diluted (1:2000) in 0.01 M borate buffer (100 μl/well). Incubate plates overnight at 2-7° C.; covered; equilibrate plates for approximately 20-30 minutes at room temperature. Wash plates 3× with PBS-T (1×PBS+0.05% Tween-20) at 300 μl/well with the Titertek M96 plate washer or equivalent. Block plates with 5% skim milk in PBS-T (Blocking Buffer) (200 μl/well) and incubate plates for 2 hours at 37° C. Wash plates 1× with PBS-T at 300 μl/well with the Titertek M96 plate washer or equivalent. Dilute NDV allantoic fluid 1:200 in Blocking Buffer. Add 100 μl/well of the diluted antigen to the plate, and incubate plates for 1 hour at 37° C. Wash plates 3× with PBS-T at 300 μl/well with the Titertek M96 plate washer or equivalent. Dilute test chicken serum samples (1:50). Dilute negative control serum (1:50) (Neg. Control 27 November 2000). Dilute positive control serum (1:10,000 or 1:20,000) (SPAFAS Chicken α-NDV serum). All serum samples are diluted in Blocking Buffer. Add 100 μl/well of Negative Control Serum to Column 1 Rows B-G; add 200 μl/well of Positive Control Serum to Columns 2-3 Row A; add 200 μl/well of Test Serum Samples to Rows A appropriate columns. This allows 4 samples per plate with 8 dilutions per sample. Add 100 μl/well of Blocking Buffer to all remaining wells; Serially two-fold dilute the Positive Control Serum and the Test Serum Samples down the plate. Dilute the samples down the plate from Row A to Row H, discarding the remaining 100 μl/well. Incubate plates for 1 hour at 37° C. and wash plates 3× with PBS-T at 300 μl/well with the Titertek M96 plate washer or equivalent. Dilute the Goat α-Chicken IgG (H&L)-HRP (1:3000) in Blocking Buffer. Add 100 μl/well of the diluted conjugate to each plate; once the conjugate is added to the plates, equilibrate ABTS substrate at RT in the dark. Incubate plates for 1 hour at 37° C.; wash plates 3× with PBS-T at 300 μl/well using with the Titertek M96 plate washer or equivalent. Add 100 μl/well of pre-warmed ABTS substrate to each plate. Leave 2-3 minutes between plates. Read plates at dual wavelength 405/490 nm on the Tecan Sunrise plate reader or equivalent when the first dilution of the positive control reaches an absorbance of between 0.7 and 1.0.

Serum ELISA AIV-HA

Coat plates with Rabbit α-HA pooled antiserum diluted (1:1000) in 0.01 M borate buffer and incubate plates overnight at 2-7° C., covered. Equilibrate plates for approximately 20-30 minutes at room temperature and wash plates 3× with PBS-T (PBS Stock+0.05% Tween-20) at 300 μl/well using the Titertek M96 plate washer or equivalent. Block the plates with 5% skim milk in PBS-T (Blocking Buffer) (200 μl/well) and incubate plates for 1 hour at 37° C. Wash plates 1× with PBS-T at 300 μl/well using the Titertek M96 plate washer or equivalent. Dilute inactivated T/W/68 AIV Allantoic Fluid (1:100) in Blocking Buffer and add 100 μl/well of the diluted antigen to the plate; incubate plates for 1 hour at 37° C. Wash the plates 3× with PBS-T at 300 μl/well using the Titertek M96 plate washer or equivalent. Dilute test chicken serum samples (1:50); dilute negative control serum (1:50); dilute positive control serum (1:25600) (USDA/SEPRL Chicken α-AIV (T/W/68 antiserum) in Blocking Buffer. Add 100 el/well of Negative Control Serum to Column 1 Rows B-G; add 200 μl/well of Positive Control Serum to Columns 2-3 Row A; add 200 μl/well of Test Serum Samples to Row A in appropriate columns; add 100 μl/well of Blocking Buffer to all remaining wells. Serially two-fold dilute the Positive Control Serum and the Test Serum Samples down the plate, discarding the remaining 100 μl/well, and incubate plates for 1 hour at 37° C. Wash plates 3× with PBS-T (300 μl/well) using the Titertek M96 plate washer or equivalent. Dilute Goat α-Chicken IgG (H&L)-HRP (1:3000) in Blocking Buffer and add 100 μl/well of the diluted conjugate to each plate. Once the conjugate is added to the plates, equilibrate ABTS substrate at RT in the dark. Incubate plates for 1 hour at 37° C. and wash plates 3× with PBS-T at 300 l/well using the Titertek M96 plate washer or equivalent. Add 100 μl/well of equilibrated ABTS substrate to each plate; allow 2-3 minutes interval between plates. Read plates at dual wavelength 405/490 nm on the Tecan Sunrise plate reader or equivalent when the first dilution of the positive control reaches an absorbance of between 0.7 and 1.0.

Serum ELISA IBDV-VP2

Blood and serum collection was performed as described above for Serum ELISA for LT. For serum ELISA, chicken anti-IBDV was adsorped to plates 1.0 μg/ml in 0.1M borate buffer at pH 6.5 with incubation overnight at 2-7° C. The plates were washed 3× with PBS-T and then blocked for 1 hour at 37° C. with 3% skim milk in PBS-T. Two hundred μl of the chicken serum sample diluted in blocking buffer was added to Row A and 100 μl of blocking buffer was added to the remaining rows. Starting dilution for serum was 1:10 in blocking buffer unless specified otherwise. After two-fold serial dilution of the serum samples, the plates were incubated 1 hour at 37° C. and then washed 3× in PBS-T. Goat anti-chicken conjugate labeled with HRP was added and incubated 1 hour at 37° C. Plates were washed and 100 μl of ABTS was added and incubated until the positive control provided a 0.7 to 1.0 absorbance at 405/492 dual wavelength using a Tecan Sunrise Plate reader. Data was transported and displayed using Tecan Magellan Software as described above for the Serum ELISA for LT.

### EXAMPLE 7

### Hemagglutination and Hemagglutination Inhibition of Red Blood Cells

Hemagglutination. Chicken red blood cells in Alsevers solution (CRBC) were obtained from Colorado Serum (L#8152). To prepare a 1% solution of CRBCs, five ml was transferred to a 15 ml conical tube and centrifuged at 250×g for 10 minutes. The supernatant and buffy coat were pipetted from the RBC pellet; the pellet was washed twice by resuspending in 1×DPBS (Dulbecco's Phosphate Buffered Saline) (L# 003435E JRH) and centrifuged 250×g for 10 minutes. The pellet was resuspended to 1% (v/v) in DPBS. To confirm the concentration of the suspension, 400 μl was transferred to 1.6 ml of deionized water and cells lysed by mixing vigorously. The OD₅₄₀ was between 0.4-0.5. The 1% solutions were stored at 2-7° C. until used. To test hemagglutination, a 96 well U-bottom dish (Falcon) was first sprayed with Static Guard™ and blotted onto paper towels. Virus samples were prediluted in DPBS 1:2 and 50 μl of DPBS were placed to each well of the 96-well dish. The diluted virus was added to the first row and then serially diluted 2-fold for the desired number of dilutions per virus sample. 50 μl of 1% CRBC was added to each well and the plate was mixed for 20 seconds at 600 rpm. The plate was placed on wet paper towels and incubated until the CRBCs in the control wells (DPBS and CRBCs at 1:1 ratio) pellet to the bottom of the plate, or for at least 1 hour at 2-7° C. The end point was the dilution of the last well in the series that provides 100% agglutination.

Hemagglutination inhibition (HAI). Virus was prediluted in DPBS to provide 4-8 HA units per 50 μl (based on titering the virus described above). A separate plate was set up using 25 μl of DPBS per well in columns 1 and 3-12; 25 μl of serum was added per well in column 1 and 3; serum in column 3 was serially diluted 2 fold through 10 wells. The pretitered virus (25 μl) was then added in all wells column 3-12 and mixed 20 seconds at 600 rpm; the plate was allowed to incubate at room temperature for 1 hour+/−15 minutes. Fifty ill of 1% CRBC was then added per well, mixed 20 seconds at 600 rpm and incubated in a humidifying chamber overnight at 2-7° C. for AIV or 1-2 hours at 2-7° C. for NDV. The titer of the serum is the last well in the series dilution that inhibits agglutination 100%.

### EXAMPLE 8

### Antigenicity in Rabbits

To test whether the plant derived protein in the immunoprotective particles extracted in non-detergent buffers, as described above, would generate antibody in animal species both HA and HN protein were prepared and inoculated into rabbits. New Zealand White rabbits 3 months of age were inoculated with plant-derived HA-AIV or HN-NDV according to the dose schedule provided in Table 4. For the primary inoculation the antigen was mixed with Complete Freund's Adjuvant (CFA) and Incomplete Freund's Adjuvant was used for all booster inoculations. The antibody titers induced by both proteins are provided in Table 5. The results indicate that after two inoculations, HAI antibody titers were induced by both proteins demonstrating that plant derived immunoprotective particles of the present invention prepared from late phase growth in NT-1 cells induce antibody in mammals that can recognize native protein. The data suggests that plant-derived HN and HA have features shared with native derived HN and HA protein. The titers of the plant-derived AIV-HA inoculated rabbits were higher than those induced by the NDV-HN plant-derived protein. This may be significant since the AIV/HA protein had lower overall activity of biological activity (hemagglutination) per unit of AIV-HA protein than NDV-HN (Table 4 column 4).

### EXAMPLE 9

### Efficacy and Biological Activity of Expressed Antigens: Challenge in Poultry and In Vitro Cytotoxicity

Challenge Trials for Newcastle disease virus (NDV). To examine the efficacy of the plant derived HN protein were inoculated in two separate trials using birds that were 2 days of age and 10 days of age. The dose concentrations for Trial #16 used for these studies are provided in Table 6. All vaccine inoculum was formulated with soluble fraction of NT-1 cells grown 15-20 days in shaker flasks at 25° C. Adjuvant used in both trials was MPL-TDM from Corixa, Inc. Intranasal groups were given MPL alone as the adjuvant.

Two-day old SPF chicks were inoculated by various routes using biologically active (hemagglutination positive) NDV-HN protein derived from NT-1 with the amount of HN protein per inoculation shown in Table 6. The serological and challenge results of this trial are provided in Table 7. All control groups responded as expected. Birds not receiving NDV-HN antigen in the inoculum had 100% mortality, whereas, control birds receiving 20 μg of native NDV by SQ had 100% survival. In the experimental treatment using plant derived HN antigen groups there was 75% protection in group #3 (SQ inoculation without adjuvant) and 80% protection in group #4 (SQ inoculation with adjuvant). The remaining treatment groups, which were inoculated by IN and oral routes, had 100% mortality. However, in group 6 two birds had a delay in mortality, indicating that these birds may have been sensitized to vaccination (see Table 10, row 14) and require a different formulation to enhance efficacy when administered by this route. In a subsequent trial (#18), 10-day old SPF birds were inoculated with doses as described in schedule Table 8. One control group (#3), a non-vaccinated non-challenged treatment was used to show that the housing and facility had no adverse affects on general health of the chickens. Control groups in this trial also responded as expected. Since birds from both trials were challenged at the same facility, treatment group # 2 served as a positive control for both Trials 16 (Table 7) and 18 (Table 9). In the remaining groups, all of which were inoculated SQ with HN derived from NT1 cells, there was 100% survival in group #7, 80% survival in each of groups 5 and 6, and 60% survival in group 4 (see Table 9).

Challenge Trial for Avina Influenza (AIV). In a separate study broiler chicks were vaccinated with plant derived hemagglutinin protein (HA). The plant derived HA protein gene sequence of avian influenza virus (AIV) strain A/turkey/Wisc/68 (H₅N₉) was transformed into NT1 cells using the vector system described for NT1 CHN-18. The NT1 line designation for transgenic plant cell production of HA-AIV protein was CHA-13. Chicks were received from the hatchery at 3 days of age and 10 birds were randomly placed in cages for each treatment group. The dose for each treatment group is shown in Table 11. The birds were given three doses at day 0, 14 and 28 of the study, blood samples were collected at Day 0, 21, 35 and 45. Serum from each blood sample was analyzed for HAI titer; at day 35 the birds were shipped to the Southeast Poultry Research Laboratory in Athens, Ga. where they were challenged with virulent AIV (Chicken/Pennsylvania/1370/1983). The data provided in Table 11 indicate that a 30 μg dose of HA protein derived from CHA-13 NT1 lines provided a seroconversion to HAI positive titer after only two doses of the vaccine preparation. Upon challenge all vaccinated groups showed protection against AIV; a Challenge Score of 50 or above indicates disease or clinical pathology. All groups regardless of formulation showed a very similar titer to native AIV upon challenge indicating a memory response to native virus induced by plant derived protein (column 4, Table 11).

Challenge Trial for infectious bursa disease virus (IBDV). The above trials indicate that two types of glycoproteins derived from transgenic plant cells according to the present invention are highly efficacious in that they can protect target species from virulent challenge. In an additional study the gene for a non-glycosylated structural protein VP2 from IBDV was transformed into NT-1 cells with similar vector and promoter construction as that for CHN-18 and CHA-13. The resulting transfected cell described here was designated CVP2-002. In this study SPF chickens were vaccinated on days 7, 21 and 35 post hatch with NT-1 control cell lysate, cell lysate from transgenic NT cells expressing the VP2 protein from IBDV (transformation event CVP2-002) and Vi Bursa K⁺ V commercially available inactivated Infectious Bursal Disease Virus (IBDV) vaccine (Lohman Animal Health). NT-1 control cells were expanded in a 10 L fermenter and passage 6 CVP2-002 cells were expanded in shaker flasks. Cells were harvested at 10-14 days post plant and lysed by passing through a Microfluidics 110L microfluidizer fitted with a 100 μm Z configuration interaction chamber at 18,000 PSI. The resulting cell lysates were clarified by centrifugation at 2000×g. The clarified supernatant was concentrated by lyophilization. Vaccines were formulated with adjuvant and the VP2 concentration of each vaccine was determined by ELISA prior to vaccination. Table 12 describes vaccine formulation, route of administration and VP2 concentrations at each vaccination date. Blood samples were collected on days 21, 35 and 42 post hatch and tested for antibody response in a serology ELISA and for neutralizing antibody titer in an IBDV Serum Neutralization (SN) assay. Birds were challenged by bilateral intraocular instillation of 50 EID₅₀ embryo derived STC strain of IBDV. Birds were euthanized 10 days post challenge. Bursa to body weight (BBW) and spleen to body weight (SBW) ratios were determined for each bird. Bursal tissue from each bird was fixed in formalin and scored for IBDV associated lesions as indicated by bursal follicle depletion. BBW ratios, SBW ratios and bursal lesion scores were compared to non-challenge control birds. Birds were scored as protected from challenge if there was no statistical significant difference in the BBW between the unchallenged and control. Table 13 summarizes the serology and challenge results for each vaccine group, which indicate that the plant derived VP2 antigen produces a serological response that is actually greater (by ELISA) than the conventional killed IBD vaccine. Furthermore, protection against challenge as measure by BBW indicates that the plant-derived VP2 protects as well as the conventional killed IBD vaccine (compare row 4 to row 10 Table 13).

Cytotoxicity of Heat Labile Toxin in Y1 Adrenal Cells. Y1 adrenal cells from mice were purchased from ATCC(CCL-79, L#1353400). The cell vial was thawed at 37° C. and placed into a 25 cm² T-flask (Corning) containing 10 ml of growth media consisting of 15% donor horse serum (Quad-5 L# 2212), 2.5% fetal bovine serum (JRH L# 7N2326), 1% glutamax-1 (Gibco L# 1080323) in F-12K media (Gibco L# 1089716). Cells were incubated at 37° C. in 5% CO₂. Cells were maintained in this growth media at each passage and for LT and CT cytotoxicity assays. To assay, the cells were passed onto 96 well cell culture plates (Nunc) and allowed to reach 80% confluence. LT toxin was diluted to 1 μg/ml in F-12K growth media. The toxin was further diluted by two fold serial dilutions on a 96 well microtiter plate by adding 100 ul of the prediluted sample to row A of the plate. Two fold serial dilutions were then made by transferring 50 ul of the sample in row A to 50 μl of growth media in the next well. Each dilution of the sample was transferred to 1-4 wells of Y1 adrenal cells depending on availability of samples or cells. The end point titer of LT toxin is the amount of protein required to obtain 50% cytotoxicity (cell death) (EC₅₀) (Guidry, et. al. 1997; Donta, et. al. 1974). The toxins used were the G192, R72, and K63 single amino acid gene substitution mutants of heat labile toxin of Escherichia coli (E. coli) produced in NT-1 transgenic cell lines SLT105, SLT107 and SLT102, respectively. The three mutant forms of LT toxin have been reported to be toxin in bio-assays in vitro and in vivo with the G192, R72, K63 providing approximately 10-fold, 100-fold and 1000-fold less toxicity than wild type LT toxin, respectively (Rappuoli, et. al. 1999. Immunology Today 20: 293-500). The concentration of LT mutants made in plants were compared with toxicity of LT wild type toxin from E. coli in the Y1 adrenal assay (see Table 14), the results indicate that the plant derived toxin follows similar levels of sensitivity of seen for same mutants derived from E. coli. Furthermore, because quantitation of LT toxin is determined by G1 ganglioside capture ELISA method the plant-made toxins mimic fully assembled holotoxin (Guidry, et. al. 1993. Inf. and 1 mm. 65: 4943-4950).

Mucosal Delivery of Plant Made Immunoprotective Particles from CHA-13 and CHN-18.

To determine whether non-replicating plant derived antigens delivered on mucosal surfaces are potent immunoprotective material, a bird study was performed by inoculating antigen directly onto the eye and nasal mucosal surfaces using formulations prepared by microfluidization to create homogenous emulsions for inoculation. Seven day old broiler chicks were randomly distributed in cages (5 birds per group) and inoculated with antigen ranging from 2.6-16.7 μg/bird (see Table 15). The birds were given three doses of vaccine at day 0, day 14 and day 21 days of the study, birds were 42 days of age at the end of the study. The antigens included HN derived from CHN-18 transgenic plant cells, HA derived from CHA-13 transgenic plant cells, and inactivated avian influenza virus (AIV) derived from allanotic fluid of infected chick embryos. The antigen preparations were made as described in Example 3 above. Five separate adjuvants were used in various formulations and immune response was determined by serology for hemagglutination inhibition and serum ELISA (See Table 15). The results of the study are shown in Table 16. After three doses all but one formulation resulted in seroconversion in birds inoculated with the plant derived HN from CHN-18. One of the formulations resulted in seroconversion of birds inoculated with HA from CHA-13 and two formulations resulted in serocoversion of birds inoculated with inactivated AIV antigen. One adjuvant was common to all responding groups which was Quil A mixed with cholesterol. The results indicate that non-replicating antigens derived from plants provided a serological response in birds by inoculation onto mucosal surfaces.

### EXAMPLE 10

### Production and Accumulation of Expressed Protein in Transgenic Cells

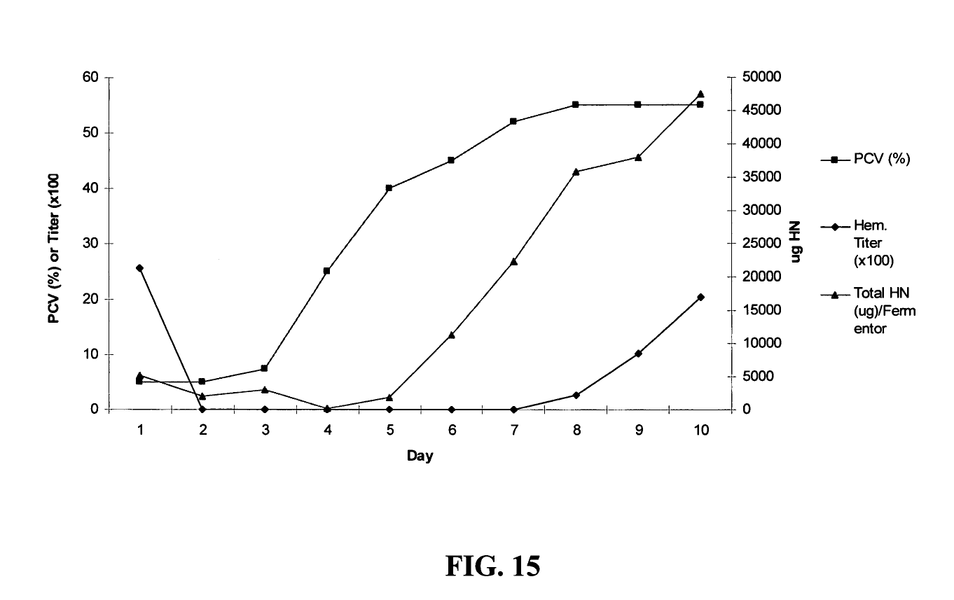

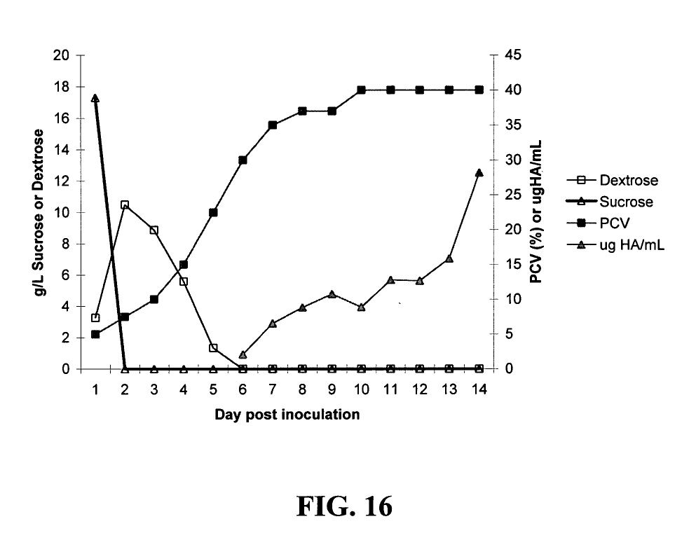

The rDNA expressed protein from transgenic cell culture grown in 10 liter bioreactors or shaker flasks is shown in FIGS. 15-18. NT-1 transgenic cell cultures producing CHN-18, CHA-13, SLT102, or CVP2-002 transgenic cells in media described above in Example 2, were harvested after 12 days of culture (stationary phase). Inoculum from the shaker flask was then transferred aseptically to a 10 liter Bioflow 3000 Fermentor (New Brunswick), containing 10 L of growth media containing 1 ml of Pluronic L61 antifoam. The cell production is performed at 25° C. with an agitation of 100 rpm and aeration at 2.5 liters per minute at 30% dissolved oxygen; cell production is performed for 9-15 days. Packed cell volume (PCV) was determined by adding 10 ml of fermentation culture to a 15 ml conical tube and centrifuging for 10 minutes at 2000×g, cell volume was then measured and evaluated as a parameter to track cell growth from inoculation day through day 10 or stationary phase of the culture. The data indicated that after about a 3 day lag there wais an exponential growth phase of the culture between day 3 and day 7, after which the culture began to reach stationary phase for each transgenic cell line analyzed regardless of the inserted gene and promoter system used. For CHN-18, the amount of measurable HN protein was tracked at each day. At day one, prior to cell new cell growth, there is a HN ELISA signal that can be extracted, which represents the amount of HN present in the inoculum harvested from the shaker flask at day 12 of culture. However, the HN is rapidly degraded and is not detected until about day 6 of the culture when the cells are reaching stationary phase and continues to accumulate in the cell after the cells have gone through stationary phase. The HN expression was followed by two different measurements, the closed triangles represent HN protein measured by quantitative ELISA and the closed squares represent hemagglutination. The quantitative ELISA is more sensitive to HN protein production and measures both monomer or polymerized HN protein, the hemagglutination measures only dimer or polymerized protein capable of agglutinating red blood cells and, thus, more protein needs to accumulate before hemagglutination activity can be determined (FIG. 15). The phenomenon of late phase production of protein is observed regardless of the protein expressed (holotoxin LT of E. coli, hemagglutinin protein (HA) of avian influenza virus; VP2 structural protein of infectious bursa disease virus, or (HN) hemagglutinin-neuraminidase protein of Newcastle Disease Virus) (See FIGS. 16, 17, and 18).

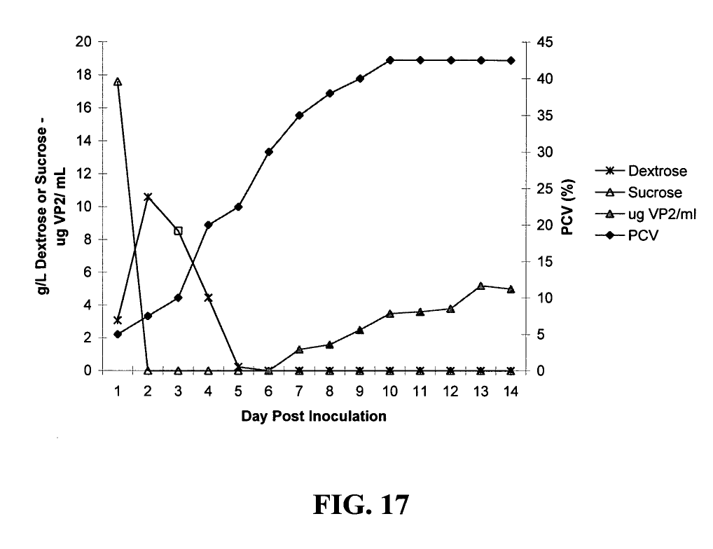

The production and growth curves for CHA-13 and CVP2-002 are illustrated in FIGS. 16 and 17, respectively. For CHA-13, growth (PCV) starts on day 2 post inoculation and enters stationary phase on day 10 post inoculation. Sucrose is consumed by day 2 post inoculation and dextrose is consumed by day 6 post inoculation. HA accumulation starts at day 6 post inoculation (mid log-growth) and increases through day 14. Cell growth starts on day 2 post inoculation and enters stationary phase on day 9 or 10 post inoculation. Sucrose is consumed by day 2 post inoculation and dextrose is consumed by day 5 post inoculation. VP2 accumulation starts at day 7 post inoculation (mid log-growth) and increases through day 14.

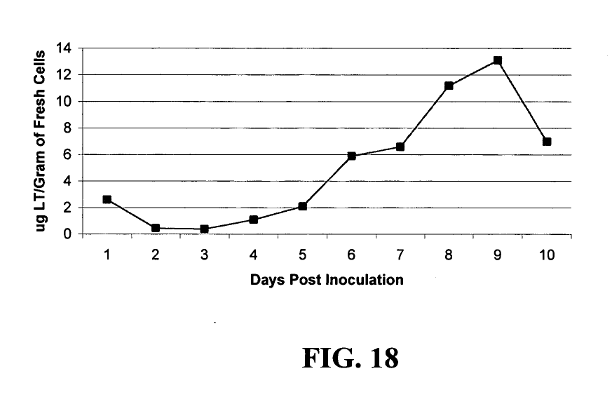

SLT102 transgenic NT-1 cell line expressing the K63 mutant for of E. coli heat labile toxin (LT) is shown in FIG. 18. The LT toxin begins to accumulate between day 5 and 6, the packed cell volume is not shown in this experiment but is similar to that for other NT-1 transgenic cell lines.

### EXAMPLE 11

### Stability of Plant Made Proteins

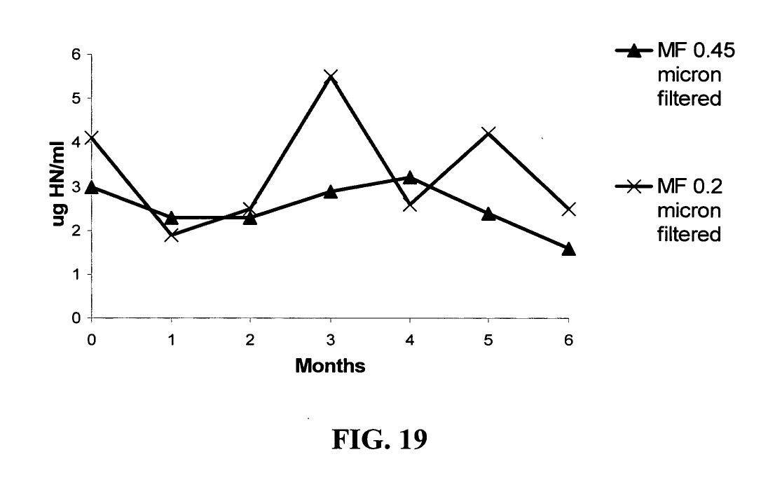

Proteins extracted from recombinant or native sources are often unstable due to proteases, glycosylases, lipases or other enzymes that co-purify with the protein and cellular components. The proteins and immunoprotective particles isolated from NT-1 cells are inherently stable and are robust to many different types of down stream processing activities. In FIG. 19, CHN-18 cells were harvested from a 10 liter fermentor in stationary phase and filtered, clarified by centrifugation, and microfluidized one time according to methods described in Example 3. The supernatants were then filtered through a 0.2 or 0.45 micron filter to remove any bacterial agents that may have been introduced during manipulation through filtration or microfluidization, no stabilizers were added to these suspensions, the stability is inherent to the proteins derived from these transgenic cells. The material was then stored at 2-7° C., 25° C. or frozen at −80° C.; the material was found to be stable at all temperatures, but the most interesting results is that when held at 25° C. (ambient temperature) the isolated proteins were found to be stable (shown in FIG. 19). Although variation in signal was seen from month to month the amount of isolated protein showed remarkable stability after several months, the half life that can be calculated from these data indicate an extrapolated half life of 8 months (0.45 micron sample) and greater than 1 year for the 0.2 micron filtered sample.

### EXAMPLE 12

### Subcellular Localizaton of Antigen

Confocal laser scanning microscopy (CLSM). Confocal laser scanning microscopy was performed to localize HN antigen in transformed MHN-41 and CHN-18 cells. Antibodies used for the localization procedure were IgG Purified Rabbit anti-HN Polyclonal (Capture Ab in HN ELISA) and HN Mab 4A—non purified from ascites fluid (Detector Ab in HN ELISA). Images were obtained from cultured plant cells using the following procedure. Cells, including non-transformed control cells (NT-Ctrl.), were spun at 1000 g×5 minutes and fixed with 3.7% formaldehyde for 15 minutes. Cells were then washed with PBS 2 times at 5 minutes for each wash. Cells were spun at 3000 g for 2 minutes (each time) and then treated with 0.5% Triton X-100 and 1% pectolyase for 15 minutes. A wash with H₂O was performed and the H₂O was replaced with methanol (−20° C.); the cells were loaded onto coated slides with different wells via a pipette and air dry (in hood 20-30 min). Wash with PBS and then block in 3% BSA/PBS for 30 min. The primary antibody (in 1% BSA-PBS-T (PBS with 0.05% Tween-20 is incubated for 1 hour at 37° C. or 1.5-2 hours at RT). Wash 3× with PBS-T. Secondary antibody, labeled with Cy5/Cy2 (1:100) is incubated for 1 hour RT, the slides are washed 3× with PBS-T and mounted.

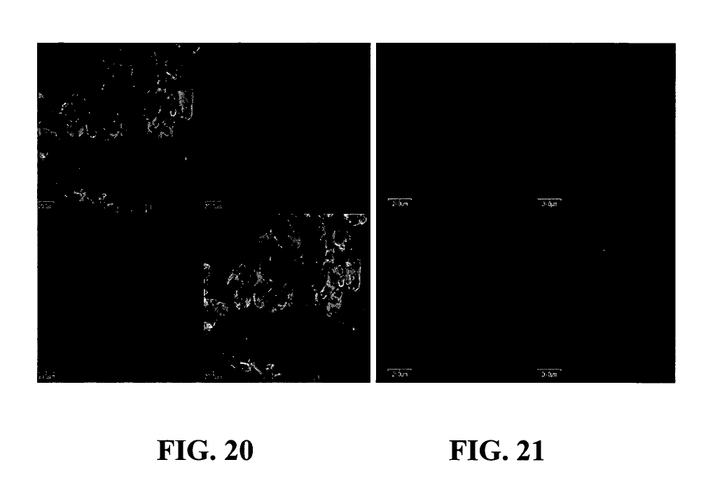

No staining was observed in NT control cells with the Rb anti-HN polyclonal or HN Mab 4A. Bright staining throughout the cell cytoplasm (but not the nucleus) of stationary phase cells was observed throughout the MHN-41 line with both HN specific antibodies. (See FIGS. 20 and 21).

Electron Microscopy. To establish where expressed protein is accumulating, transgenic plant cells were harvested after 10 days in culture and prepared for thin sectioning and immunogold label as follows. The immunogold labeling was done using purified IgG from rabbits that had been immunized with HN protein purified from Newcastle disease virus preparations of allantoic fluid taken from 10-day-old infected chicken egg embryos. For defining morphology features, cell suspensions were fixed in 3% glutaraldehyde in 0.1M phosphate buffer (pH 6.8) for 3 hours. Then they were washed in phosphate buffer for 1 hour with 4 changes of buffer. Cells were post-fixed in 2% osmium tetroxide in phosphate buffer for 1 hour. Cells were dehydrated in ascending ethanol series (25%, 50%, 75% 95% and 100%, 15 minutes each step) and propylene oxide. Cells were left in propylene oxide/Epon 812 mixture overnight before they were embedded in Epon 812 and polymerized at 60° C. for 2 days. Sections were cut with LKB Ultrotome III, stained with 2% aqueous uranyl acetate and lead citrate, and examined with Hitachi 7500 transmission electron microscope operated at 80 kV.

For immunogold staining, glutaraldehyde-fixed cells were dehydrated in ascending ethanol series after phosphate buffer washing (with 0.02 M glycine added). Cells were then infiltrated with LR White Resin overnight and finally embedded and polymerized at 50° C. for 24 hrs.

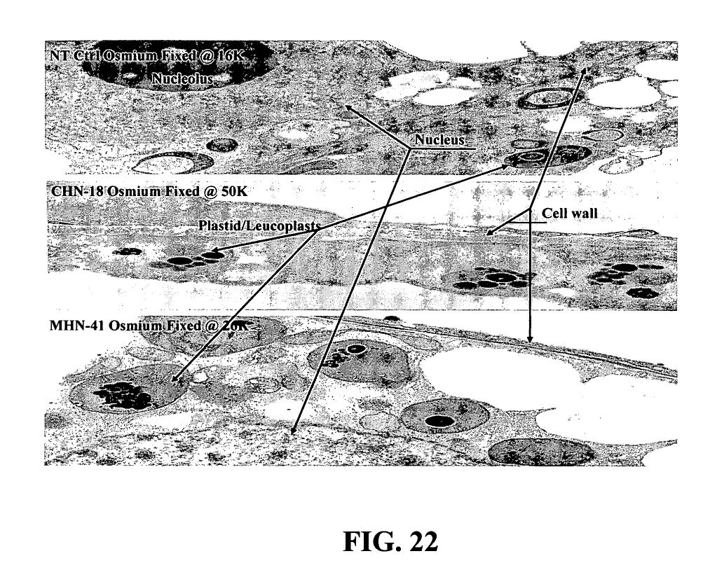

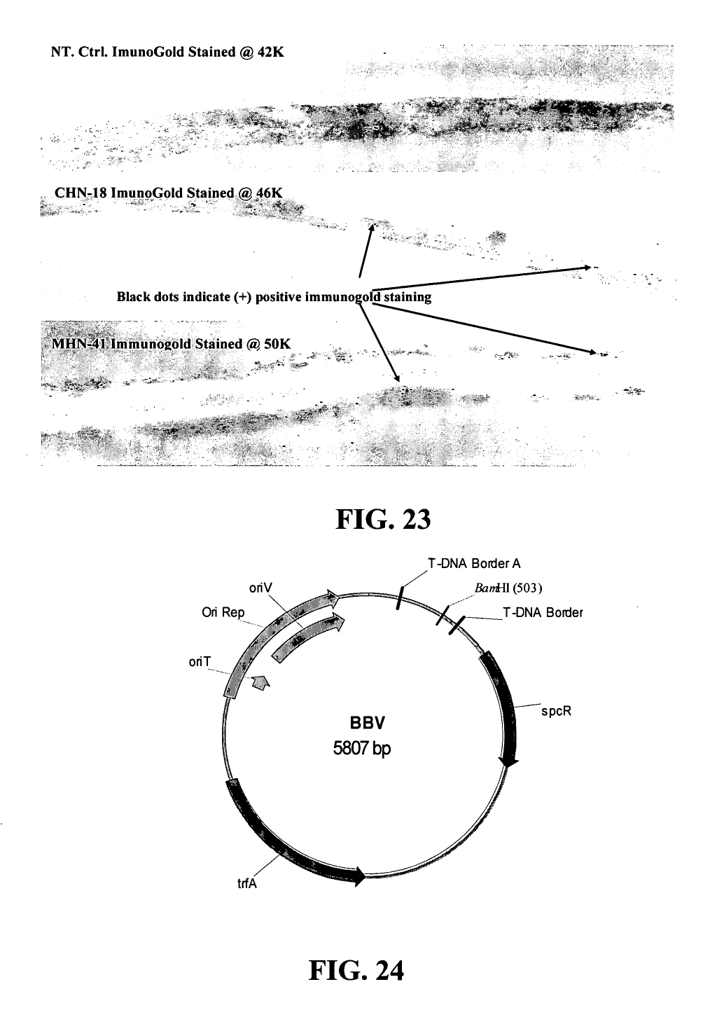

Sections mounted on nickel grids were incubated with 1% solution of bovine serum albumin in PBS buffer at pH 7.4 for 20 minutes to block non-specific sites. Cells were then incubated with primary antibody (dilution 1:150 in PBS) for 2 hours at room temperature. Then rinsed with PBS-BSA 6 times (3 minutes each) and incubated with colloidal gold (15 nm) conjugated with goat-anti-rabbit AB (diluted 1:150 in PBS) for 2 hours at room temperature. After rinsing the cells in PBS 4×5 minutes and water 2×1 minutes, the grids were stained with uranyl acetate for 5 minutes. The EM pictures indicated two major differences between control cells and transgenic cells expressing HN protein, first plastid/leucoplasts show dark granules accumulating in the transgenic cells but not the control cells (FIG. 22) and, secondly immunogold stain granules can be seen accumulating near the cell wall of the transgenic cells but not the control cells (FIG. 23). Typically gene products expressed in a host cell will occur during exponential growth of the cell and can be generally be stained in the endoplasmic reticulum, golgi apparatus, and other protein synthesizing substructure in the cell. Together with the confocal imaging described in FIGS. 19 and 20 the data indicate that the protein is being produced and deposited in the cell membranes and cell walls, but no protein can be seen accumulating in the nucleus, chloroplasts, mitochondria, endomplasmis reticulum or golgi apparatus by electron microscopy. The electron microscopy demonstrates that the late stationary phase cells have a enlarged vacuole and compressed cytoplasmic and nucleus. The confocal imaging suggests that the protein is compressed against the cytoplasmic cell wall and membranes throughout the cell.

It is unusual that the protein production and accumulation in the cell would not be apparent until late exponential and stationary phase when the cell is no longer in active metabolism. The rapid loss of expressed protein signal (24 hours) at inoculation of cells in growth flasks or fermentors (see FIG. 15) indicates that the cell is using the protein as a nitrogen source, when the cell has completed active growth, storage of nitrogen sources (proteins) can then occur. This phenomenon is a unique feature to transgenic proteins produced in plant cell culture described in the above examples. The location of the protein near the cell wall and membranes helps to explain the unexpected ability to isolate the protein easily with mechanical disruption. The ability of the each protein class to be easily isolated in stable and efficacious format is also not expected. Although any single protein can often be made in any foreign host system chosen to study recombinant DNA expression, many proteins especially trans-membrane bound glycoproteins, are often made at low levels and one host system does not express two glycoproteins in the same manner. In the cell culture transgenic system described here at least five classes of proteins (an enzyme, type 1 viral glycoprotein, type 2 viral glycoprotein, LT toxin, and a structural non-glycosylated protein VP2 have been successfully expressed to similar levels in the same host system. Furthermore, the proteins accumulate in late stationary phase regardless of the class of protein, transcriptional cassette or promoter system and can be easily removed from the cell using the same physical or mechanical disruption methods. Regardless of the protein class expressed by these transgenic cells, each protein has been successfully isolated in stable form that is biologically active.

### EXAMPLE 13

### Infectious Bursal Disease Plant-Optimized VP2 Antigen Gene

The viral causative agent of Infectious Bursal Disease (IBD) Virus (or IBDV) has a bipartite RNA genome (J. Virol. (1979) 32:593). Full-length RNA1 is translated into a polyprotein that is processed into peptides VP2, VP3, and VP4. In silico reverse transcription of the genomic RNA can be performed to obtain a DNA sequence corresponding to the protein coding capacity of the native RNA. The 1359 base pairs (bp) of the derived DNA sequence of the Ehime91 (E/91) strain of IBVD which encode the native E/91 VP2 protein are available as GenBank Accession AB024076. Analysis of this sequence revealed the presence of several sequence motifs that are thought to be detrimental to optimal plant gene expression, as well as a non-optimal codon composition (see, for example, U.S. Pat. No. 5,380,831). To improve production of the recombinant VP2 protein in monocots as well as dicots, a “plant-optimized” DNA sequence (SEQ ID No: 11) was developed that encodes a protein (disclosed herein as SEQ ID No: 12) essentially identical to the native E/91 VP2 protein, except for the addition of single Isoleucine, Alanine, and Valine residues at the carboxy-terminal end of the native E/91 protein. Codons for these additional amino acids were included based on the report of J. Caston et al. (J Virol (2001) 75:10815) which indicates that the optimal VP2 processing site for VP2 capsid assembly occurs after amino acid position 456, rather than 453, which is the last amino acid encoded by IBD strain UK661 (GenBank Accession NC_{—}004178) VP2 sequence is identical to that of E/91, with the exception of position 451 (Leu vs. Ile). Thus, amino acids 454, 455, and 456 (Ile, Ala, Val) were derived from the UK661 strain for engineering of a 456 amino acid VP2 gene. The VP2 protein encoded by the native E/91 sequence and the VP2 protein encoded by the plant-optimized coding region are 99.3% identical, differing only at amino acid numbers 454, 455, and 456. In contrast, the derived DNA of the native E/91 VP2 coding region and the plant-optimized DNA are only 80.3% identical.

Foreign genes are integrated into plant chromosomes in random fashion, and the possibility exists that any particular integration site may be one that is conducive to adventitious production of new, abberant proteins from gene control elements and open reading frames flanking the integration site. To help eliminate the production of these unwanted and possibly detrimental proteins, additional bases which encode translation termination codons in all six possible reading frames were included downstream of the VP2 coding region (“universal terminator”; disclosed as SEQ ID No: 13). To enable subsequent cloning steps, bases comprising the recognition sites for three restriction enzymes are included in this useful sequence.

### EXAMPLE 14

### Construction of Basic Binary Vector pDAB2423 for Expression of Infectious Bursal Disease Plant-Optimized VP2 Antigen Gene in Plant Cells

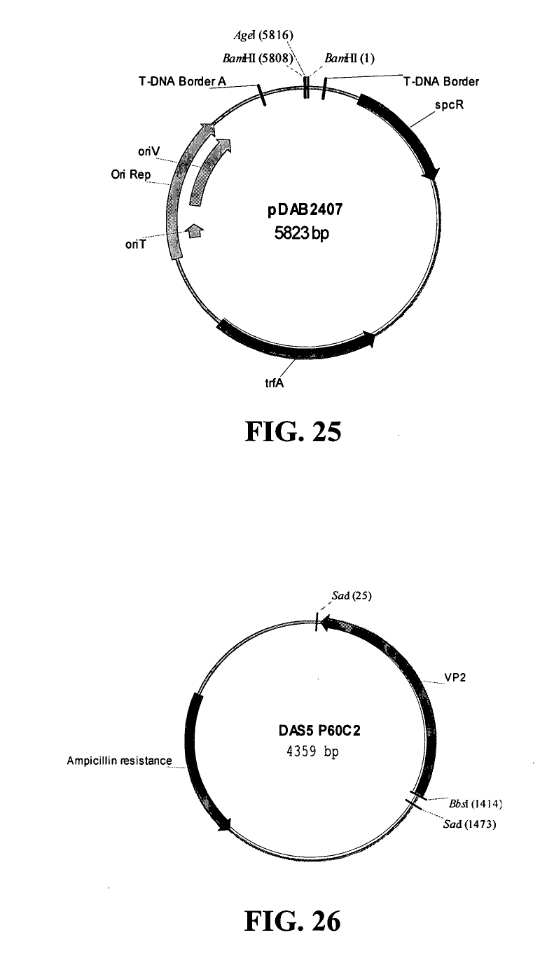

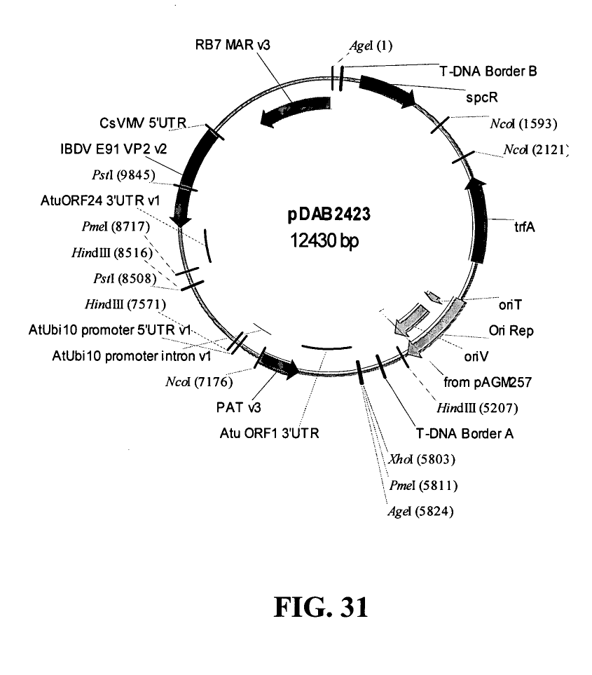

A dicot expression vector containing the plant-optimized nucleotide sequence of IBD VP2 gene (SEQ ID NO: 11) was constructed. Using a basic binary vector (BBV) backbone (FIG. 24), a modification was made at the unique BamHI site with addition of an AgeI linker. The new binary vector (pDAB2407, FIG. 25) allowed for AgeI/AgeI ligation of a VP2 and selectable marker expression cassette between the T-DNA borders (pDAB2423, FIG. 31).

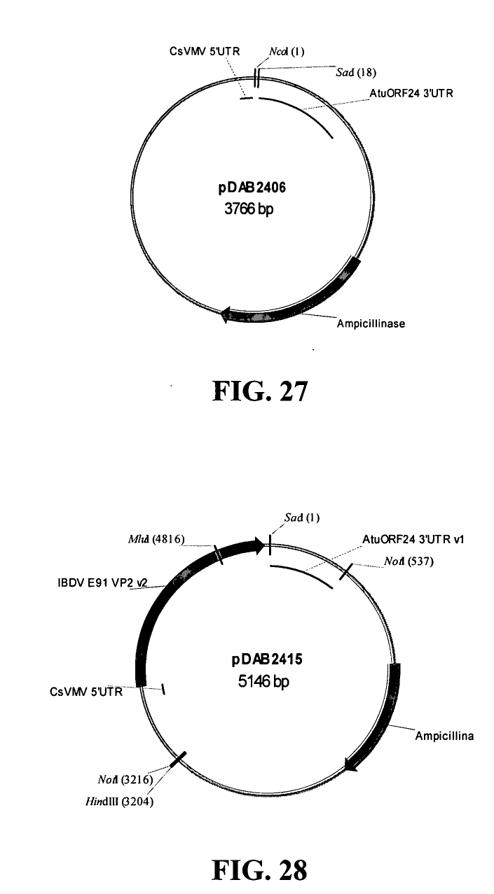

The expression cassette was assembled by excising the synthesized VP2 sequence from DAS5 P60C2 (FIG. 26, PICOSCRIPT, Houston, Tex.) with BbsI and SacI restriction enzymes. pDAB2406 (FIG. 27), encoding the CsVMV promoter and Agrobacterium tumifaciens (Atu) ORF24 3′UTR (GenBank accession number X00493), was cut with NcoI and SacI. The pDAB2406 backbone and the VP2 insert fragments were ligated at the NcoI and SacI sites of pDAB2406, resulting in pDAB2415 (FIG. 28). Ligated DNA was transformed into E. coli DH5α Competent Cells (Invitrogen) and screening was done for positive clones. Positive clones were identified by restriction analysis, using HindIII×MluI enzymes and confirmed with sequencing across insert/vector junctions.

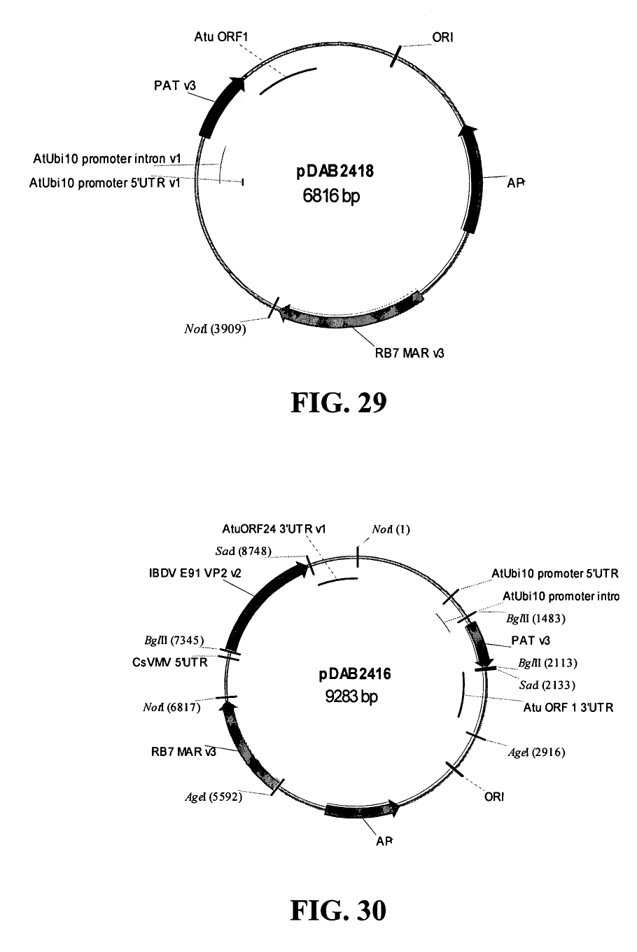

Once a pDAB2415 subclone was confirmed, the plasmid was cut with NotI to isolate the CsVMV/VP2/ORF24 fragment. pDAB2418 (FIG. 29), encoding RB7 MAR element (U.S. Pat. No. 5,773,689; U.S. Pat. No. 5,773,695; U.S. Pat. No. 6,239,328, WO 94/07902, and WO 97/27207) and the selectable marker, PAT, regulated by Arabidopsis thaliana (At) Ubiquitin 10 (Ubi10) promoter (Plant J. 1997. 11(5):1017; Plant Mol. Biol. 1993. 21(5):895; Genetics.1995. 139(2):921) and Atu ORF1 3′ UTR. (U.S. Pat. No. 5,428,147; Plant Molecular Biology. 1983. 2:335; GenBank accession number X00493), was linearized with NotI. The pDAB2418 backbone and pDAB2415 insert fragments were isolated on a gel, purified, and ligated at the NotI sites, resulting in pDAB2416 (FIG. 30). Ligated DNA was transformed into E. coli and colonies were screened by restriction enzyme digests with BglII and SacI. Further confirmation of the positive subclone was done by sequencing across the NotI junctions. pDAB2416 plasmid was then cut with AgeI to remove the MAR/CsVMV/VP2/ORF24//Ubi10/PAT/ORF1 expression cassette from the vector backbone. pDAB2407 was also cut with AgeI to linearize the binary vector and the appropriate fragments were ligated to form pDAB2423. After transformation of the ligated DNA, colonies were screened using HindIII and XhoI digests. Of 30 colonies picked, 12 were positive. One positive clone was further analyzed by restriction digests using NcoI, PmeI, and PstI enzymes. For final verification, the clone was filly sequenced between the T-DNA borders.

TABLE 1
Comparison of extraction methods on hemagglutination
activity of plant-derived HN

| | | | | Ext. buffer | DPBS |
|---|---|---|---|---|---|
| | DPBS | Ext. buffer | DPBS | F/T | F/T |
| | Sonic. | Sonic. | Sonic. | Sonic. | Sonic. |
| Sample | 1.5 min | 1.5 min | 15 sec | 15 sec | 15 sec |
| pCHN-18-NT-1 | ≦2 | 256 | 4096 | 1024 | 1024 |
| pCHA-47-NT-1 | ≦2 | — | 64 | 16 | 16 |
| NT-1 | ≦2 | ≦2 | ≦2 | ≦2 | ≦2 |
| Native NDV¹ | 256 | 512 | 128 | nd | nd |

¹Native NDV was sonicated for 2 minutes.
Ext. buffer - 50 mM sodium ascorbate, 1 mM EDTA, 1 mM PMSF, and 0.1% Triton X-100 pH 7.2;
DPBS—Dulbecco's phosphate buffered saline;
sonic.—sonication;
F/T—freeze-thaw;
nd—not done for this experiment.

TABLE 2
Comparison of hemagglutination inhibition (HAI) activity
of plant-derived HN and native virus

| | | | Hemagglutination |
|---|---|---|---|
| | | | Inhibition Titer |
| | HN Concentration | | (chicken anti-NDV |
| Sample | ELISA | Hemagglutination Titer | polyclonal antibody) |
| NDV allantoic | 20 ug/ml | 4¹ | 4096 |
| fluid (native) | | | |
| NT control cell | None | ≦2 | ≦8 |
| pCHN-7-NT-1 | 1.5 μg/g fresh weight | >64 | 512 |
| CHN-18-NT-1 | 12 μg/g fresh weight | ≧4096 | 1024 |
| CLT-101-14-NT-1 | None | ≦2 | ≦8 |

¹Stock virus is 4HA units, equating to a 1:4 dilution of the stock virus. This is the concentration of virus used to titer antibody, the endpoint dilution of antibody that will interfere with 4 HA units of virus is considered to be the HAI titer of the antibody preparation.

TABLE 3
Comparison of hemagglutination of cell extracts
of CHN-18 transgenic cells using various
pressures for microfluidization

| Treatment | S/N or Pellet | HA Titer |
|---|---|---|
| Sonicated | S/N | 2048 |
| Sonicated | Pellet | 1024 |
| MF 4500 PSI | S/N | 4096 |
| MF 4500 PSI | Pellet | 4096 |
| MF 4500 PSI, retreat | S/N | 4096 |
| pellet 4500 PSI | | |
| MF 4500 PSI, retreat | Pellet | 768 |
| pellet 4500 PSI | | |
| MF 6000 PSI | S/N | 8192 |
| MF 6000 PSI | Pellet | 8192 |
| MF 8000 PSI | S/N | 8192 |
| MF 8000 PSI | Pellet | 8192 |
| MF 10000 PSI | S/N | 8192 |
| MF 10000 PSI | Pellet | 8192 |
| MF 12000 PSI | S/N | 8192 |
| MF 12000 PSI | Pellet | 8192 |
| MF 14000 PSI | S/N | 8192 |
| MF 14000 PSI | Pellet | 8192 |
| MF 18000 PSI | S/N | 32,768 |
| MF 18000 PSI | Pellet | 8192 |

MF—microfluidized;
PSI—pounds per square inch;
S/N—supernatant

TABLE 4
Dose levels for inoculation into rabbits

| | | ELISA Results | | Hemagglutination |
|---|---|---|---|---|
| | Hemagglutination | (μg | BCA TSP¹ | units |
| Sample | Endpoint Titers | protein/ml) | Results (mg/ml) | per μg protein |
| NT Control | ≦2 | 0.00 | 1.44 | 0 |
| CHN-7 | 2048 | 13.53 | 2.48 | 3027 |
| CHN-18 | 1024 | 9.21 | 4.10 | 2223 |
| CHA-13 | 32 | 3.80 | 6.15 | 168 |
| CHA-47 | 16 | 2.88 | 5.25 | 111 |

¹All NT-1 samples were provided from non freeze-dried material.
BCA—bicinchoninic acid, primary component of Pierce Chemical BCA protein assay kit;
TSP—total soluble protein.
CHN-7 and CHN-18 are two separate transgenic cell lines expressing the HN protein from NDV, CHA-13 and CHA-47 are two separate transgenic cell lines expressing the HA protein of AIV.

TABLE 5
Serology results from rabbits inoculated with AIV-HA and NDV-HN protein
derived from transgenic plant cells CHA-13 and CHN-18

| | Sample | | | | | | | | |
|---|---|---|---|---|---|---|---|---|---|
| | Number | Pre-Bleed | 6 week | 8 week | 10 week | Pre-Bleed | 6 week | 8 week | 10 week |
| | | NDV HAI Titers | | | | NDV ELISA Titers | | | |
| Treatment with | | | | | | | | | |
| NDV-HN | | | | | | | | | |
| S/N from NT Control Cells | 2723 | ≦8 | ≦8 | ≦8 | ≦8 | 0 | 0 | 0 | 0 |
| S/N from CHN - 18 Cells | 2724 | ≦8 | 23 | 23 | 23 | 0 | 815 | 554 | 888 |
| S/N from CHN - 18 Cells | 2725 | ≦8 | 11 | 23 | 23 | 0 | 0 | 585 | 591 |
| | | AIV HAI Titers | | | | AIV ELISA Titers | | | |
| Treatment with | | | | | | | | | |
| AIV-HA | | | | | | | | | |
| S/N from NT Control Cells | 2723 | ≦8 | ≦8 | ≦8 | ≦8 | <25 | <25 | <25 | <25 |
| S/N from CHA - 13 Cells | 2728 | ≦8 | 362 | 362 | 362 | <25 | 25600 | 25600 | 25600 |
| S/N from CHA - 13 Cells | 2729 | ≦8 | 181 | 181 | 11 | <25 | 3200 | 3200 | <25 |

S/N—supernatant;
HAI—hemagglutination inhibition serum titer

TABLE 6
Dose levels used per inoculation for poultry Trial #16

| | μg HN/Bird | | |
|---|---|---|---|
| Group | Day 0 | Day 14 | Day 21 |
| NT Control (SQ) | 0 | 0 | 0 |
| NDV All. Fluid (SQ) | 20 | 20 | 20 |
| CHN-18 (SQ) | 150 | 230 | 180 |
| CHN-18 (IN) | 6 | 14 | 14 |
| CHN-18 (OG) | 114 | 240 | 135 |
| CHN-18 (OG + OF) | 114og + | 240OG/ | 2366 OF¹ |
| | 700OF¹ | 1400OF¹ | 135OG + |
| Average | 3590 | 5810 | 6025 |
| hemagglutination | | | |
| units per μg HN | | | |

¹Dose based on expression of hemagglutinin/neuraminidase (HN) per wet weight cells;
IN—intranasal;
SQ—subcutaneous;
OG—oral gavage;
OF—on feed mixtures.

TABLE 7
Serology and challenge results for CHN-18 from poultry
trial #16 (NDV challenge)

| | | NDV HAI titers | | | | | NDV ELISA titers | | | |
|---|---|---|---|---|---|---|---|---|---|---|
| | Sample | Day | Day | | Pre | Post | Day | Pre | Post | Surv. |
| Treatment | Number | 14 | 21 | Day 28 | Chall. | Chall. | 28 | Chall. | Chall. | Chall. |
| 1. Control NT Cells | 924 | ≦8 | ≦8 | ≦8 | ≦8 | na | 0 | 0 | na | No |
| SQ | 1061 | ≦8 | ≦8 | ≦8 | ≦8 | na | 0 | 0 | na | No |
| | 1073 | ≦8 | ≦8 | ≦8 | ≦8 | na | 0 | 0 | na | No |
| | 1077 | ≦8 | ≦8 | ≦8 | ≦8 | na | 0 | 0 | na | No |
| | 1081 | ≦8 | ≦8 | ≦8 | ≦8 | na | 0 | 0 | na | No |
| 2. NDV HN allantoic | 1063 | 11 | 1448 | 2896 | 724 | 724 | 11956 | 9245 | 7294 | Yes |
| fluid SQ | 1068 | 11 | 1448 | 1024 | 724 | 362 | 9216 | 7639 | 6122 | Yes |
| | 1072 | 45 | 1448 | 1448 | 724 | 362 | 11592 | 7500 | 5937 | Yes |
| | 1083 | 23 | 724 | 724 | 181 | 91 | 5697 | 4919 | 3011 | Yes |
| | 1089 | 45 | 1024 | 1448 | 362 | 181 | 15181 | 7449 | 6085 | Yes |
| 3. CHN-18 | 797 | 23 | 45 | 45 | ≦8 | 2896 | 0 | 0 | 19036 | Yes |
| SQ | 1066 | ≦8 | 16 | 45 | ≦8 | 724 | 450 | 0 | 10587 | Yes |
| | 1085 | ≦8 | ≦8 | 23 | ≦8 | na | 0 | 0 | na | No |
| | 1095 | ≦8 | 23 | 45 | ≦8 | 724 | 436 | 0 | 10043 | Yes |
| 4. CHN-18 | 1067 | 11 | 45 | 45 | ≦8 | 181 | 592 | 0 | 4912 | Yes |
| MPL/TDM adjuvant | 1080 | ≦8 | 181 | 181 | 45 | 181 | 1911 | 871 | 5048 | Yes |
| SQ | 1093 | 11 | 45 | 45 | ≦8 | ≦8 | 0 | 0 | 0 | Yes |
| | 1094 | 11 | 91 | 91 | 11 | 11 | 747 | 199 | 0 | Yes |
| | 1098 | ≦8 | 23 | 45 | ≦8 | na | 0 | 0 | na | No |
| 5. CHN-18 | 796 | ≦8 | ≦8 | ≦8 | ≦8 | na | 0 | 0 | na | No |
| IN | 925 | ≦8 | ≦8 | ≦8 | ≦8 | na | 0 | 0 | na | No |
| | 1065 | ≦8 | ≦8 | ≦8 | ≦8 | na | 0 | 0 | na | No |
| | 1084 | ≦8 | ≦8 | 8 | ≦8 | na | 0 | 0 | na | No |
| | 1092 | ≦8 | ≦8 | ≦8 | ≦8 | na | 0 | 0 | na | No |
| 6. CHN-18 + MPL | 921 | ≦8 | ≦8 | ≦8 | ≦8 | na | 0 | 0 | na | No |
| adjuvant | 923 | ≦8 | 11 | ≦8 | ≦8 | na | 0 | 0 | na | No |
| IN | 1069 | ≦8 | ≦8 | ≦8 | ≦8 | na | 0 | 0 | na | No |
| | 1074 | ≦8 | 11 | ≦8 | ≦8 | na | 0 | 0 | na | No |
| | 1088 | ≦8 | 11 | 8 | ≦8 | na | 0 | 0 | na | No |
| 7. CHN-18 | 723 | ≦8 | ≦8 | ≦8 | ≦8 | na | 0 | 0 | na | No |
| Oral gavage | 1062 | ≦8 | ≦8 | ≦8 | ≦8 | na | 0 | 0 | na | No |
| | 1075 | ≦8 | 8 | ≦8 | ≦8 | na | 0 | 0 | na | No |
| | 1079 | ≦8 | 8 | ≦8 | ≦8 | na | 0 | 0 | na | No |
| | 1086 | ≦8 | ≦8 | ≦8 | ≦8 | na | 0 | 0 | na | No |
| 8. CHN-18 + MPL/ | 1070 | ≦8 | ≦8 | ≦8 | ≦8 | na | 0 | 0 | na | No |
| TDM adjuvant | 1082 | ≦8 | ≦8 | ≦8 | ≦8 | na | 0 | 0 | na | No |
| Oral gavage + On feed | 1091 | ≦8 | ≦8 | ≦8 | ≦8 | na | 0 | 0 | na | No |
| | 1097 | ≦8 | ≦8 | ≦8 | ≦8 | na | 0 | 0 | na | No |
| | 1100 | ≦8 | ≦8 | ≦8 | ≦8 | na | 0 | 0 | na | No |

All birds receive 10² EID₅₀ (egg infectious dose) Texas GB strain of NDV. Birds were challenged 24 days post last vaccination. Bird numbers bolded had a delayed onset to mortality see Table 9.
HAI—hemagglutination inhibition serum titers;
na—not applicable

TABLE 8
Dose levels of antigen used per inoculation for Trial #18

| | μg HN/Bird (Subcutaneous) | | |
|---|---|---|---|
| Group | Day 0 | Day 14 | Day 21 |
| NT Control | 0 | 0 | 0 |
| Inactivated NDV from | 20 | 20 | 20 |
| allantoic fluid | | | |
| CHN-18 (low dose) | 20 | 20 | 20 |
| CHN-18 (high dose) | 150 | 100 | 100 |
| Average | 3590 | 2625 | 2625 |
| hemagglutination | | | |
| units per μg HN | | | |

TABLE 9
Serology and challenge results CHN-18 from Trial #18 (NDV challenge)

| | | NDV HAI titer | | | | NDV ELISA titers | | | | |
|---|---|---|---|---|---|---|---|---|---|---|
| | Sample | | | Pre | Post | | | Pre | Post | Surv. |
| Treatment | Number | Day 21 | Day 28 | Chall. | Chall. | Day 21 | Day 28 | Chall. | Chall. | Chall. |
| 1. Control allantoic | 1026 | ≦8 | ≦8 | ≦8 | na | 0 | 0 | 0 | na | No |
| fluid | 1027 | ≦8 | ≦8 | ≦8 | na | 0 | 0 | 0 | na | No |
| | 1028 | ≦8 | ≦8 | ≦8 | na | 0 | 0 | 0 | na | No |
| | 1029 | ≦8 | ≦8 | ≦8 | na | 0 | 0 | 0 | na | No |
| | 1030 | ≦8 | ≦8 | ≦8 | na | 0 | 0 | 0 | na | No |
| 2. NDV HN allantoic | 1031 | 362 | 362 | 181 | 91 | 9177 | 6937 | 5533 | 3551 | Yes |
| fluid | 1032 | 362 | 724 | 181 | 91 | 12393 | 16533 | 7909 | 6080 | Yes |
| 20 μg/dose | 1033 | 362 | 724 | 181 | 181 | 8622 | 15291 | 6766 | 6362 | Yes |
| | 1034 | 724 | 362 | 181 | 181 | 7875 | 10071 | 6487 | 5822 | Yes |
| | 1035 | 1448 | 724 | 181 | 181 | 9681 | 16133 | 7537 | 6539 | Yes |
| 3. Control tobacco | 1036 | ≦8 | ≦8 | ≦8 | ≦8 | 0 | 0 | 0 | 0 | n/c |
| | 1037 | ≦8 | ≦8 | ≦8 | ≦8 | 0 | 0 | 0 | 0 | n/c |
| | 1038 | ≦8 | ≦8 | ≦8 | ≦8 | 0 | 0 | 0 | 0 | n/c |
| | 1039 | ≦8 | ≦8 | ≦8 | ≦8 | 0 | 0 | 0 | 0 | n/c |
| | 1040 | ≦8 | ≦8 | ≦8 | ≦8 | 0 | 0 | 0 | 0 | n/c |
| 4. CHN-18 | 1041 | ≦8 | 11 | ≦8 | 1448 | 0 | 0 | 0 | 14042 | Yes |
| 20 μg/dose | 1042 | ≦8 | 23 | ≦8 | 2896 | 0 | 0 | 0 | 19263 | Yes |
| | 1043 | 8 | 32 | ≦8 | na | 0 | 0 | 0 | na | No |
| | 1044 | ≦8 | ≦8 | ≦8 | na | 0 | 0 | 0 | na | No |
| | 1045 | 23 | 23 | ≦8 | 1024 | 0 | 0 | 0 | 11770 | Yes |
| 5. CHN-18 | 1046 | 11 | 23 | ≦8 | 1448 | 0 | 674 | 0 | 11243 | Yes |
| 20 μg/dose + MPL/ | 1047 | 11 | 23 | ≦8 | ≦8 | 0 | 963 | 0 | 0 | Yes |
| TDM | 1048 | 32 | 23 | ≦8 | na | 396 | 757 | 0 | na | No |
| emlusion adjuvant | 1049 | 23 | 45 | ≦8 | 362 | 0 | 804 | 0 | 6239 | Yes |
| | 1050 | 11 | 11 | ≦8 | 1448 | 0 | 398 | 0 | 15948 | Yes |
| 6. CHN-18 | 1051 | 45 | 91 | 23 | 181 | 1096 | 1137 | 565 | 4547 | Yes |
| 250 μg/dose | 1052 | 45 | 45 | ≦8 | 181 | 1166 | 998 | 0 | 7376 | Yes |
| | 1053 | 23 | 23 | ≦8 | 2896 | 0 | 0 | 0 | 16712 | Yes |
| | 1054 | 11 | 23 | ≦8 | na | 646 | 838 | 0 | na | No |
| | 1055 | 32 | 45 | 23 | 91 | 705 | 563 | 448 | 4902 | Yes |
| 7. CHN-18 | 1056 | 45 | 45 | 11 | 23 | 746 | 948 | 174 | 926 | Yes |
| 250 μg/dose + | 1057 | 11 | 45 | 11 | 724 | 556 | 892 | 0 | 11542 | Yes |
| MPL/TDM | 1058 | 23 | 45 | 23 | 724 | 780 | 1588 | 630 | 9915 | Yes |
| emulsion adjuvant | 1059 | 32 | 91 | 23 | 91 | 2004 | 3090 | 1016 | 4690 | Yes |
| | 1060 | 45 | 45 | 11 | 181 | 916 | 1522 | 448 | 6620 | Yes |

All birds received 10² EID₅₀ (egg infectious dose) Texas GB strain of NDV, except group 1, which received 10⁴ EID₅₀ Texas GB strain of NDV and group 3, which was the non-challenge control. Birds were challenged 31 days post last vaccination. Hemagglutination inhibition (HAI) titers are reported as a mean titer from three replicas.

TABLE 10
Death on days after challenge for Trial #16 and #18 (NDV challenge)

| Group # | Treatment | Route | Chall. | D1 | D2 | D3 | D4 | D5 | D6 | D7 | D8 | D9 | D10 | D11-14 | % Surv. |
|---|---|---|---|---|---|---|---|---|---|---|---|---|---|---|---|
| 1-018 | Control allantoic fluid | SQ | 10⁴ | 0 | 0 | 3 | 2 | — | — | — | — | — | — | — | 0 |
| | | | EID₅₀ | | | | | | | | | | | | |
| 2-018 | NDV HN allantoic fluid - 20 μg/ | SQ | 10² | 0 | 0 | 0 | 0 | 0 | 0 | 0 | 0 | 0 | 0 | 0 | 100 |
| | dose | | EID₅₀ | | | | | | | | | | | | |
| 3-018 | Control tobacco | SQ | None | 0 | 0 | 0 | 0 | 0 | 0 | 0 | 0 | 0 | 0 | 0 | 100 |
| 4-018 | CHN-18 derived - 20 μg/dose | SQ | 10² | 0 | 0 | 0 | 0 | 1 | 0 | 0 | 0 | 1 | 0 | 0 | 60 |
| | | | EID₅₀ | | | | | | | | | | | | |
| 5-018 | CHN-18 derived - 20 μg/dose + | SQ | 10² | 0 | 0 | 0 | 0 | 0 | 1 | 0 | 0 | 0 | 0 | 0 | 80 |
| | MPL/TDM Emulsion adjuvant | | EID₅₀ | | | | | | | | | | | | |
| 6-018 | CHN-18 derived 250 μg/dose | SQ | 10² | 0 | 0 | 0 | 0 | 0 | 0 | 1 | 0 | 0 | 0 | 0 | 80 |
| | | | EID₅₀ | | | | | | | | | | | | |
| 7-018 | CHN-18 derived 250 μg/dose + | SQ | 10² | 0 | 0 | 0 | 0 | 0 | 0 | 0 | 0 | 0 | 0 | 0 | 100 |
| | MPL/TDM Emulsion adjuvant | | EID₅₀ | | | | | | | | | | | | |
| 1-016 | Control tobacco | SQ | 10² | 0 | 0 | 0 | 4 | 1 | — | — | — | — | — | — | 0 |
| | | | EID₅₀ | | | | | | | | | | | | |
| 2-016 | NDV HN allantoic fluid | SQ | 10² | 0 | 0 | 0 | 0 | 0 | 0 | 0 | 0 | 0 | 0 | 0 | 100 |
| | | | EID₅₀ | | | | | | | | | | | | |
| 3-016 | CHN-18 derived | SQ | 10² | 0 | 0 | 0 | 0 | 0 | 0 | 0 | 0 | 0 | 0 | 1 | 75 |
| | | | EID₅₀ | | | | | | | | | | | | |
| 4-016 | CHN-18 derived + MPL/TDM | SQ | 10² | 0 | 0 | 0 | 0 | 0 | 0 | 1 | 0 | 0 | 0 | 0 | 80 |
| | Emulsion adjuvant | | EID₅₀ | | | | | | | | | | | | |
| 5-016 | CHN-18 derived | IN | 10² | 0 | 0 | 0 | 2 | 3 | — | — | — | — | — | — | 0 |
| | | | EID₅₀ | | | | | | | | | | | | |
| 6-016 | CHN-18 derived + MPL adjuvant | IN | 10² | 0 | 0 | 0* | 3 | 0 | 0 | 0 | 2 | — | — | — | 0 |
| | | | EID₅₀ | | | | | | | | | | | | |
| 7-016 | CHN-18 derived | oral | 10² | 0 | 0 | 0 | 4 | 1 | — | — | — | — | — | — | 0 |
| | | gav. | EID₅₀ | | | | | | | | | | | | |
| 8-016 | CHN-18 derived + MPL/TDM | oral | 10² | 0 | 0 | 0 | 2 | 3 | — | — | — | — | — | — | 0 |
| | adj | | EID₅₀ | | | | | | | | | | | | |

*One bird in a group of 5 was not recorded for this date

TABLE 11
Serology and challenge results for CHA-13 (AIV challenge)

| | Day 21 | Day 34 | | |
|---|---|---|---|---|
| | AIV | AIV | Day 45 | Challenge |
| Treatment Group | HAI¹ | HAI | AIV HAI | Score² |
| 1. NT Control (Challenged) | ≦1 | ≦1 | 76 | 69 |
| 2. NT-1 Control | ≦1 | ≦1 | ≦1 | 6 |
| (Unchallenged) | | | | |
| 3. Inactivated AIV | 3 | 18 | 1351 | 0 |
| (20 μg/dose) from Allantoic | | | | |
| Fluid in Corixa MPL/TDM | | | | |
| Emulsion³ | | | | |
| 4. CHA-47 derived antigen | 7 | 14 | 799 | 9 |
| (2 μg/dose) in Corixa | | | | |
| MPL/TDM Emulsion | | | | |
| 5. CHA-47 derived antigen | 3 | 10 | 790 | 24 |
| (2 μg/dose) | | | | |
| 6. CHA-47 derived antigen | 25 | 4 | 1218 | 10 |
| (30 μg/dose) in Corixa | | | | |
| MPL/TDM emulsion | | | | |
| 7. CHA-47 derived antigen | 7 | 2 | 624 | 8 |
| (30 μg/dose) | | | | |

¹Hemagglutination inhibition (HAI) titer is reported as 50% endpoint with two replicas, a value of 1 designates background.
²Challenge score was determined by a composite rating of conjunctivitis = 1; depression = 2; ataxia = 3; paralysis/torticollis = 4; death/euthanasia = 5.
³Corixa adjuvant monophosphoryl lipid A (MPL) Trehalose dicorynomycolate (TDM)

TABLE 12
Dose level used per inoculation for poultry trial

μg VP2/bird

| Group¹ | Day 7 | Day 21 | Day 35 |
|---|---|---|---|
| NT Control (SQ) | 0 | 0 | 0 |
| Vi Bursa K + V | ND | ND | ND |
| nCVP2-002 (SQ) | 17.9 | 18.4 | 17.4 |
| nCVP2-002 w/Drakeoil | 7.8 | 14.0 | 14.2 |
| Emulsion (SQ) | | | |
| nCVP2-002 (OG) | 17.9 | 18.4 | 17.4 |
| nCVP2-002 (IB) | 17.9 | 18.4 | 17.4 |
| nCVP2-002 w/50 μg/ | 18.5 | 18.4 | 17.9 |
| dose CGI (SQ) | | | |
| nCVP2-002 w/LAP (SQ) | 8.9 | 17.0 | 17.5 |
| nCVP2-002 (SQ, OG) | 17.9 | 18.4 | 17.4 |
| nCVP2-002 (SQ) | 9.0 | 9.2 | 8.7 |

¹SQ—Subcutaneous;
OG—Oral Gavage;
IB—Intrabursal;
SQ OG - 1^{st} dose subcutaneous 2^{nd} and 3^{rd} doses Oral Gavage;
Drakeoil Emulsion - nCVP2-002 with equal volume of 5% Drakeoil, 1% Tween 80, 0.33% Span 80;
CGI—Purified Cellcap derived Chicken Gamma Interferon;
LAP—nCVP2-002 with equal volume of Lecithin Acrylic Polymer oil in water emulsion

TABLE 13
Serology and Challenge Results from CVP2-002 Poultry Trial (IBDV challenge)

| | SN Titer Range¹ | | | Serology ELISA Titer Range² | | | p-value |
|---|---|---|---|---|---|---|---|
| Group | Day 21 | Day 35 | Day 42 | Day 21 | Day 35 | Day 42 | BBW³ |
| NT Control (SQ) | ≦11 | ≦11 | ≦11 | <100 | <100 | <100-200 | na |
| (unchallenged) | | | | | | | |
| NT Control (SQ)(challenged) | ≦11 | ≦11 | ≦11 | <100 | <100 | <100 | 0.0002 |
| Vi Bursa K + V | ≦11-152 | 1,722-23,170 | 2,048 < 46,431 | <100-800 | 3200-25,600 | 4,000-32,000 | 0.8697 |
| nCVP2-002 (SQ) | ≦11-64 | <11-1,722 | 76-9,742 | <100-1600 | 400-6400 | 3,200-64,000 | 0.0747 |
| nCVP2-002 w/Drakeoil | ≦11-181 | <11-512 | 16-1,722 | <100-1600 | <100-12,800 | 200-32,000 | 0.0223 |
| Emulsion (SQ) | | | | | | | |
| nCVP2-002 (OG) | ≦11 | ≦11 | ≦11 | <100 | <100 | <100-400 | 0.00002 |
| nCVP2-002 (IB) | ≦11 | ≦11-64 | ≦11-32 | <100-100 | <100 | <100 | 0.0001 |
| nCVP2-002 w/50 μg/ | ≦11-45 | ≦11-1,722 | 45-1,448 | <100-800 | 800-25,600 | 1,600-32,000 | 0.1748 |
| dose CGI (SQ) | | | | | | | |
| nCVP2-002 w/LAP (SQ) | ≦11-64 | 362-13,777 | 861-9,742 | 100-1600 | 400-51,200 | 6,400-256,000 | 0.3349 |
| nCVP2-002 (SQ, OG) | ≦11 | ≦11 | ≦11 | <100 | <100 | <100 | 0.0007 |
| nCVP2-002 (SQ) | ≦11-45 | ≦11-362 | 19-1,448 | <100-400 | 200-6400 | 400-16,000 | 0.2361 |

¹Serum neutralization (SN) titer range is the lowest to highest values for serum titers for the group of birds per treatment.
²Serum ELISA titer range is the lowest to highest value for serum titers for the group of birds per treatment.
³Bursa to body weight (BBW) are measured for each bird within a treatment group, by comparing each challenged group to the unchallenged group (#1), a statistical difference is generated providing a p-value; a p-value greater than 0.05 is considered to be protective.
na—not applicable

TABLE 14
Y1 mouse adrenal cell cytotoxicity using various
heat labile toxin preparations

| | LTB¹ | LT² | Toxicity Y1 |
|---|---|---|---|
| | μg/gram | μg/gram | Adrenal |
| | filtered | filtered | cells |
| Source | cells | cells | Pg³/EC₅₀ |
| SLT102 NT-1 | 8-12 | 0.4-0.6 | No toxicity |
| Transgenic cell | | | observed |
| K63 mutant | | | |
| SLT107 NT-1 | 8-9 | 0.8-1.0 | 21 |
| Transgenic cell | | | |
| R72 mutant | | | |
| SLT105 NT-1 | 20-30 | 0.1-0.2 | 0.9 |
| Transgenic cell | | | |
| G192 mutant | | | |
| LT holotoxin (E. coli) | na | 1900 μg/ml | 0.1 |

¹Each antigen is assayed using the ganglioside quantitation capture ELISA as described in Example 6.
²LT concentration represents the quantity of LTA protein binding to LTB, which is captured by the ganglioside, using LTA specific antibody.
³Conentration based on LTB quantitation of toxin using LTB specific antibody.
LT—heat labile toxin;
LTA—A subunit of heat labile toxin;
LTB—B subunit of heat labile toxin

TABLE 15
Mucosal delivery (intranasal and ocular) of plant
derived and native antigen; dose
administered per treatment group

| | | | μg antigen/ |
|---|---|---|---|
| Treatment | Antigen¹ | Adjuvant² | Dose³ |
| 1-1 | CHN-18 (20Jan04) | Quil A/Chol. | 11.4 |
| 2-1 | CHN-18 (20Jan04) | LAP | 11.4 |
| 3-1 | CHN-18 (20Jan04) | LAP in Oil in Water | 12.9 |
| 4-1 | CHN-18 (20Jan04) | Quil A/Chol + LAP | 11.4 |
| 5-1 | CHN-18 (20Jan04) | LAP + E. coli LT | 11.4 |
| 6 | CHA-13 (22Jan04) | Quil A/Chol. | 25.4 |
| 7 | CHA-13 (22Jan04) | LAP | 25.4 |
| 8 | CHA-13 (22Jan04) | LAP in Oil in Water | 28.6 |
| 9 | CHA-13 (22Jan04) | Quil A/Chol + LAP | 25.4 |
| 10 | CHA-13 (22Jan04) | LAP + E. coli LT | 25.4 |
| 11 | Inact. AIV | Quil A/Chol. | 17.9 |
| 12 | Inact. AIV | LAP | 17.9 |
| 13 | Inact. AIV | LAP in Oil in Water | 20.2 |
| 14 | Inact. AIV | Quil A/Chol + LAP | 17.9 |
| 15 | Inact. AIV | LAP + E. coli LT | 17.9 |
| 16⁴ | CHN-18 SQ | Oil in Water | 2.9 |
| 17 | None (Non Vx. Control) | None | None |

¹Antigens used for the study included CHA-13 and CHN-18 plant derived antigen along with inactivated avian influenza virus (Inact. AIV), the date denotes harvest date from 10 liter fermentor and designates batch number.
²Adjuvants used for treatments were all administered by intranasal IN and ocular route by dropping 0.05 ml per eye and 0.2 ml in each nostril.
LAP—lecithin acrylic polymer;
Quil A is saponin from the bark of a tree (Quillia saponaria);
LT—heat labile toxin from E. coli;
chol.—cholesterol;
oil-Drakeol mineral oil.
³Dose level administered was determined by using dilution or combinations of antigen taken from a quantitative ELISA for bulk antigen prior to assembly of vaccine with adjuvant.
⁴Positive control sample was delivered by SQ-subcutaneous inoculation

TABLE 16
Serological response to mucosal delivered vaccines
delivery (intranasal and ocular) of plant derived and native
antigen; dose administered per treatment group

| | | | | Birds |
|---|---|---|---|---|
| | | | HAI titer | responding per |
| Treatment | Antigen¹ | Adjuvant² | range³ | #treated |
| 1-1 | CHN-18 (20Jan04) | Quil A/Chol. | 8-256 | 3/5 |
| 2-1 | CHN-18 (20Jan04) | LAP | 16 | 2/5 |
| 3-1 | CHN-18 (20Jan04) | LAP in Oil in Water | NR | NR |
| 4-1 | CHN-18 (20Jan04) | Quil A/Chol + LAP | 16-32 | 3/5 |
| 5-1 | CHN-18 (20Jan04) | LAP + E. coli LT | 64-128 | 2/4 |
| 6 | CHA-13 (22Jan04) | Quil A/Chol. | 8-256 | 3/5 |
| 7 | CHA-13 (22Jan04) | LAP | NR | NR |
| 8 | CHA-13 (22Jan04) | LAP in Oil in Water | NR | NR |
| 9 | CHA-13 (22Jan04) | Quil A/Chol + LAP | NR | NR |
| 10 | CHA-13 (22Jan04) | LAP + E. coli LT | NR | NR |
| 11 | Inact. AIV | Quil A/Chol. | 32 | 1/5 |
| 12 | Inact. AIV | LAP | NR | NR |
| 13 | Inact. AIV | LAP in Oil in Water | NR | NR |
| 14 | Inact. AIV | Quil A/Chol + LAP | NR | NR |
| 15 | Inact. AIV | LAP + E. coli LT | 16 | 2/5 |
| 16* | CHN-18 | Oil in Water | 16-64 | 4/5 |
| 17 | None (Non Vx. Control) | None | NR | NR |

^{1,2}See Table 15 for information.
³Hemagglutination inhibition (HAI) serum titer was determined using inactivated AIV and NDV antigen both derived from allantoic fluid from chick embryos. Both antigens were used as controls for each treatment, serum titer reported was the specific response to the antigen for that treatment.
NR—no response

We claim:

1. A method for making an immunoprotective particle or a biologically active protein particle comprising the steps of:
a) transforming a plant cell with a polynucleotide encoding at least one at least one immunoprotective antigen or at least one biologically active protein;
b) culturing said transformed plant cell under conditions that allow for the proliferation of said transformed plant cell and the accumulation of said immunoprotective antigen or said biologically active protein in said plant cell;
c) collecting and washing said cultured transformed cells;
d) resuspending said washed transformed cells in a lysis buffer;
e) physically or mechanically disrupting said resuspended cells such that immunoprotective particles or biologically active protein particles are formed; and
f) separating cellular debris from said immunoprotective particles or said biologically active protein particles.

2. The method of claim 1, wherein the transformed plant cell is chosen from the group consisting of a lower plant cell, a monocot plant cell, and a dicot plant cell.

3. The method of claim 2, wherein the transformed plant cell is a tobacco cell line culture.

4. The method of claim 1 wherein said immunoprotective particle or biologically active protein particle is derived from late exponential and stationary growth phase of said transformed plant cell.

5. The method according to claim 1, wherein said physical or mechanical disruption is performed by sonication, microfluidiation or other shear-type methods, high shear rotor/stator methods, French press or other pressure methods, or homogenization.

6. The method according to claim 5, wherein said physical or mechanical disruption is performed by sonication for up to twenty seconds.

7. The method according to claim 1, wherein said method steps are performed in the absence of harsh detergents or other chemical agents that disrupt cells.

8. The method of claim 1, wherein the immunoprotective antigen is a protein from an avian virus.

9. The method of claim 1, wherein the immunoprotective antigen is chosen from the group consisting of a hemagglutinin/neuraminidase protein from Newcastle Disease Virus (NDV), a hemagglutinin protein from Avaina Influenza Virus (AIV), and a VP2 protein from Infectious Bursal Disease Virus.

10. The method of claim 1, wherein the immunoprotective antigen is selected from the group consisting of SEQ ID NO: 2, SEQ ID NO:4 and SEQ ID NO: 12.

11. The method according to claim 1, wherein said plant cell is selected from the group consisting of NT-1, BY-2, CHN-18, CHA-13, CVP2, and MHN-41.

12. The method according to claim 1, wherein said polynucleotide encodes a subunit vaccine.

13. The method according to claim 1, wherein said biologically active protein is selected from the group consisting of enzymes, toxins, cell receptors, ligands, signal transducing agents, cytokines.

14. The method according to claim 1, further comprising recovering a supernatant that contains said immunoprotective particles or biologically active protein particles that has been separated from cellular debris.

15. The method according to claim 1, further comprising isolating soluble, stable, and biologically active protein from said immunoprotective particles or biologically active protein particles.

16. The method according to claim 14, further comprising isolating soluble, stable, and biologically active protein or immunoprotective proteins from said supernatant.

17. An isolated immunoprotective particle or biologically active protein particle made according to the method of claim 1, 2, 3, 4, 5, 6, 7, 8, 9, 10, 11, 12, 13, 14, 15, or 16.

18. A composition comprising an immunoprotective particle or a biologically active protein particle according to claim 17 in admixture with one or more pharmaceutically-acceptable adjuvants, diluents, carriers, or excipients.

19. The composition of claim 18 wherein the immunoprotective particle comprises at least one immunoprotective antigen.

20. The composition of claim 19, wherein the immunoprotective antigen is chosen from the group selected from a hemagglutinin/neuraminidase protein from Newcastle Disease Virus (NDV), a hemagglutinin protein from Avaina Influenza Virus (AIV) and a VP2 protein from Infectious Bursal Disease Virus.

21. The composition of claim 20, wherein the immunoprotective antigen is selected from the group consisting of SEQ ID NO:2, SEQ ID NO:4 and SEQ ID NO: 12.

22. A method for vaccinating or immunizing an animal comprising administering to an animal or human a dose of a composition according to claim 18 in an amount sufficient to vaccinate, immunize, stimulate an immune response, stimulate the production of specific antibodies, or stimulate a cellular immune response.

23. The method according to claim 22, wherein said composition provides an immunoprotective antigen chosen from the groups selected from a a hemagglutinin/neuraminidase protein from Newcastle Disease Virus (NDV), a hemagglutinin protein from Avaina Influenza Virus (AIV) and a VP2 protein from Infectious Bursal Disease Antigen.

24. The method according to claim 22, wherein said composition is administered intramuscularly, intravenously, orally, nasally, or subcutaneously.
